(19) United States
(12) Patent Application Publication
(10) Pub. No.: US 2021/0198347 A1
(43) Pub. Date: Jul. 1, 2021

(54) ANTI-COMPLEMENT COMPONENT ANTIBODIES AND METHODS OF USE

(71) Applicant: Chugai Seiyaku Kabushiki Kaisha, Tokyo (JP)

(72) Inventors: Taku FUKUZAWA, Shizuoka (JP); Kenta HARAYA, Shizuoka (JP); Wei Shiong Adrian HO, Singapore (SG); Noriyuki TAKAHASHI, Singapore (SG); Masaru MURAOKA, Shizuoka (JP)

(21) Appl. No.: 17/046,395

(22) PCT Filed: Apr. 12, 2019
(86) PCT No.: PCT/JP2019/015919

§ 371 Date: Oct. 9, 2020

(30) Foreign Application Priority Data
Apr. 13, 2018 (JP) 2018-077527
Oct. 4, 2018 (JP) 2018-188770

(51) Int. Cl. C07K 16/18

(52) U.S. Cl. CPC C07K 16/18; A61K 2039/505

(57) ABSTRACT

The invention provides anti-complement component antibodies such as anti-C1s antibodies and anti-C1r antibodies, and methods of using the same. The invention also provides pharmaceutical formulations comprising the antibodies, and methods of treating an individual having a complement-mediated disease or disorder comprising administering the antibody to the individual. The binding specificity and C1q displacement function of the anti-C1s antibodies and anti-C1r antibodies are evaluated. Time dependent complement neutralization function and binding to native and truncated C1s or C1r proteins are also shown for the antibodies.

### TECHNICAL FIELD

The present invention relates to anti-complement component antibodies such as anti-C1s antibodies and anti-C1r antibodies, and methods of using the same.

### BACKGROUND

The C1 complex is a large protein complex which functions as the key initiator of the classical pathway cascade. The C1 complex consists of three components, C1q, C1r and C1s, which are in molar ratio of 1:2:2 respectively (NPL 1). The classical pathway is initiated when the C1 complex binds to a target that is bound by antibodies. C1q, which has 6 globular heads, mediates the binding of C1 complex to the antibodies by avidity interaction with the Fc regions. Once tightly bound to the target, C1r within the C1 complex autoactivates and become enzymatically active. The activated C1r then cleaves and activates proenzyme C1s within the C1 complex (NPL 2). Subsequently, active C1s cleave its substrates complement component C2 and C4 into C2a/C2b, and C4a/C4b fragments respectively. This leads to the assembly of C4b2a, a C3 convertase, on the target surface which cleaves C3 to form C3b. C3b in turn cleaves C5 to initiate the formation of the terminal membrane attack complex, C5b, C6, C7, C8 and C9, which lyses the target via pore formation.

Both C1s and C1r proteins have an identical domain organization, which is CUB1-EGF-CUB2-CCP1-CCP2-Serine Protease (NPL 3). The CUB1-EGF-CUB2 domains mediates the interaction between C1r and C1s to form the C1r2s2 tetramer (NPL 4), and also between C1r2s2 and C1q (NPL 5). In contrast, the CCP1-CCP2-Serine Protease domains of C1r and C1s are responsible for proteolytic cleavage of their respective substrates (NPL 6, NPL 7).The C1r2s2 tetramer interacts with the six stems in C1q through six binding sites within the CUB1-EGF-CUB2 domains of the tetramer (NPL 5).

While a properly functioning complement system defends the host against pathogens, dysregulation or inappropriate activation of the classical pathway results in a variety of complement-mediated disorders such as, and not limited to, autoimmune hemolytic anemias (AIHA), Behcet's disease, Bullous Pemphigus (BP), immune thrombocytopenia purpura (ITP) etc. Therefore, inhibition of excessive or uncontrolled activation of the classical pathway can provide clinical benefit to patients with such disorders.

HI532, an antibody which binds to the beta domain of C1s, was reported to be able inhibit the interaction of C1r2s2 with C1q (NPL 8). However, this antibody was not able to completely neutralize hemolytic activity of human serum and 30% of activity remained even after 24 hrs incubation of serum with the antibody.

Antibodies are highly attractive pharmaceuticals as they are stable in plasma, highly specific for their target, and generally exhibit good pharmacokinetic profiles. However, due to their large molecular size, the dosage of therapeutic antibodies is usually high. In the case of targets that exist in high abundance, the required therapeutic dose of antibodies is even higher. As a result, methods that improve antibody pharmacokinetics, pharmacodynamics, and antigen binding properties are attractive ways to reduce the dosage and high production costs associated with therapeutic antibodies.

It has been reported that antibodies that bind to an antigen in a pH-dependent manner (herein below also referred to as “pH-dependent antibody” or “pH-dependent-binding antibody”) enables a single antibody molecule to neutralize multiple antigen molecules (NPL 9, PTL 1). The pH-dependent antibody binds strongly to its antigen at neutral pH conditions in the plasma, but dissociates from the antigen under the acidic pH condition within the endosome of a cell. Once dissociated from the antigen, the antibody is recycled back to the plasma by FcRn receptors whereas the dissociated antigens are degraded within the lysosome of the cell. The recycled antibody is then free to bind to and neutralize antigen molecules again and this process continues to be repeated as long as the antibody remains in circulation.

### CITATION LIST

### Patent Literature

[PTL 1] WO2009/125825

### Non Patent Literature

[NPL 1] Wang et. al. Mol Cell. 2016 Jul. 7; 63(1):135-45
[NPL 2] Mortensen et. al. Proc Natl Acad Sci USA. 2017 Jan. 31; 114(5):986-991
[NPL 3] Gal et. al. Mol Immunol. 2009 September; 46(14):2745-52
[NPL 4] Almitairi et. al. Proc Natl Acad Sci USA. 2018 Jan. 23; 115(4):768-773
[NPL 5] Bally et. al. J Biol Chem. 2009 Jul. 17; 284(29):19340-8
[NPL 6] Rossi et. al. 1998 J Biol Chem. 1998 Jan. 9; 273(2):1232-9
[NPL 7] Lacroix et. al. J Biol Chem. 2001 Sep. 28; 276(39):36233-40
[NPL 8] Tseng et. al. Mol Immunol. 1997 June; 34(8-9):671-9
[NPL 9] Igawa et. al. Nat Biotechnol. 2010 November; 28(11):1203-7

### SUMMARY OF INVENTION

### Technical Problem

The invention provides anti-complement component antibodies such as anti-C1s antibodies and anti-C1r antibodies, and methods of using the same.

### Solution to Problem

In some embodiments, an isolated antibody that inhibits the interaction between C1q and C1r2s2 complex of the present invention is an antibody having a displacement function such that the antibody binds to C1qrs complex and promotes dissociation of C1q from C1qrs complex.

In some embodiments, an isolated antibody that inhibits the interaction between C1q and C1r2s2 complex of the present invention is an antibody binding to C1qrs complex on a BIACORE® chip and promotes dissociation of C1q from C1qrs complex. In further embodiments, the antibody of the present invention can be determined as an antibody having a displacement function when a value of response unit (RU) in presence of the antibody is lower than a value of response unit (RU) in the absence of the antibody as determined by a BIACORE® assay when a sufficient time passed.

In some embodiments, an isolated antibody that inhibits the interaction between C1q and C1r2s2 complex of the present invention can be determined as an antibody having a displacement function when the time point of crossover is within 60 s, 100 s, 150 s, 200 s, 500 s, 700 s, 1000 s, 1500 s, or 2000 s after the time point of the start of antibody injection as determined by a BIACORE® assay using the following conditions: The capture levels of C1r2s2 complex and C1q are at 200 resonance unit (RU) and 200 resonance unit (RU), respectively, and the antibody as an analyte is injected at 500 nM at 10 microliter (micro L)/min.

In some embodiments, an isolated antibody that inhibits the interaction between C1q and C1r2s2 complex of the present invention can be determined as an antibody having a displacement function when almost all of C1q are dissociated from C1qrs complex within 100 s, 300 s, 500 s, 700 s, 1000 s, 1500 s, 2000 s, 3000 s, 5000 s, 7000 s, or 10000 s after the time point of the start of antibody injection as determined by a BIACORE® assay using the following conditions: The capture levels of C1r2s2 complex and C1q are at 200 resonance unit (RU) and 200 resonance unit (RU), respectively, and the antibody as an analyte is injected at 500 nM at 10 micro L/min.

In some embodiments, an isolated antibody that inhibits the interaction between C1q and C1r2s2 complex of the present invention is an antibody having a neutralizing activity for human serum complement of at least 70% in an RBC assay.

In some embodiments, an isolated antibody that inhibits the interaction between C1q and C1r2s2 complex of the present invention is an antibody that specifically binds to C1s or an antibody that specifically binds to C1r.

In some embodiments, an isolated antibody that inhibits the interaction between C1q and C1r2s2 complex of the present invention is an antibody specifically binding to an epitope within a CUB1-EGF-CUB2 domain of C1s. In further embodiments, the antibody of the present invention competes for binding to the epitope with an antibody selected from the group consisting of 1)-5) below:
1) an antibody comprising the HVR-H1 sequence of SEQ ID NO: 32, the HVR-H2 sequence of SEQ ID NO: 33, the HVR-H3 sequence of SEQ ID NO: 34, the HVR-L1 sequence of SEQ ID NO: 35, the HVR-L2 sequence of SEQ ID NO: 36, and the HVR-L3 sequence of SEQ ID NO: 37,
2) an antibody comprising the HVR-H1 sequence of SEQ ID NO: 38, the HVR-H2 sequence of SEQ ID NO: 39, the HVR-H3 sequence of SEQ ID NO: 40, the HVR-L1 sequence of SEQ ID NO: 41, the HVR-L2 sequence of SEQ ID NO: 42, and the HVR-L3 sequence of SEQ ID NO: 43,
3) an antibody comprising the HVR-H1 sequence of SEQ ID NO: 44, the HVR-H2 sequence of SEQ ID NO: 45, the HVR-H3 sequence of SEQ ID NO: 46, the HVR-L1 sequence of SEQ ID NO: 47, the HVR-L2 sequence of SEQ ID NO: 48, and the HVR-L3 sequence of SEQ ID NO: 49,
4) an antibody comprising the HVR-H1 sequence of SEQ ID NO: 50, the HVR-H2 sequence of SEQ ID NO: 51, the HVR-H3 sequence of SEQ ID NO: 52, the HVR-L1 sequence of SEQ ID NO: 53, the HVR-L2 sequence of SEQ ID NO: 54, and the HVR-L3 sequence of SEQ ID NO: 55, and
5) an antibody comprising the HVR-H1 sequence of SEQ ID NO: 56, the HVR-H2 sequence of SEQ ID NO: 57, the HVR-H3 sequence of SEQ ID NO: 58, the HVR-L1 sequence of SEQ ID NO: 59, the HVR-L2 sequence of SEQ ID NO: 60, and the HVR-L3 sequence of SEQ ID NO: 61.

In some embodiments, an isolated antibody that inhibits the interaction between C1q and C1r2s2 complex of the present invention is an antibody specifically binding to an epitope within a CUB1-EGF-CUB2 domain of C1r. In further embodiments, the antibody of the present invention competes for binding to the epitope with an antibody selected from the group consisting of 6)-13) below:
6) an antibody comprising the HVR-H1 sequence of SEQ ID NO:119, the HVR-H2 sequence of SEQ ID NO: 127, the HVR-H3 sequence of SEQ ID NO: 135, the HVR-L1 sequence of SEQ ID NO: 143, the HVR-L2 sequence of SEQ ID NO: 151, and the HVR-L3 sequence of SEQ ID NO: 159,
7) an antibody comprising the HVR-H1 sequence of SEQ ID NO: 120, the HVR-H2 sequence of SEQ ID NO: 128, the HVR-H3 sequence of SEQ ID NO: 136, the HVR-L1 sequence of SEQ ID NO: 144, the HVR-L2 sequence of SEQ ID NO: 152, and the HVR-L3 sequence of SEQ ID NO: 160,
8) an antibody comprising the HVR-H1 sequence of SEQ ID NO: 121, the HVR-H2 sequence of SEQ ID NO: 129, the HVR-H3 sequence of SEQ ID NO: 137, the HVR-L1 sequence of SEQ ID NO: 145, the HVR-L2 sequence of SEQ ID NO: 153, and the HVR-L3 sequence of SEQ ID NO: 161,
9) an antibody comprising the HVR-H1 sequence of SEQ ID NO: 122, the HVR-H2 sequence of SEQ ID NO: 130, the HVR-H3 sequence of SEQ ID NO: 138, the HVR-L1 sequence of SEQ ID NO: 146, the HVR-L2 sequence of SEQ ID NO: 154, and the HVR-L3 sequence of SEQ ID NO: 162,
10) an antibody comprising the HVR-H1 sequence of SEQ ID NO: 123, the HVR-H2 sequence of SEQ ID NO: 131, the HVR-H3 sequence of SEQ ID NO: 139, the HVR-L1 sequence of SEQ ID NO: 147, the HVR-L2 sequence of SEQ ID NO: 155, and the HVR-L3 sequence of SEQ ID NO: 163,
11) an antibody comprising the HVR-H1 sequence of SEQ ID NO: 124, the HVR-H2 sequence of SEQ ID NO: 132, the HVR-H3 sequence of SEQ ID NO: 140, the HVR-L1 sequence of SEQ ID NO: 148, the HVR-L2 sequence of SEQ ID NO: 156, and the HVR-L3 sequence of SEQ ID NO: 164,
12) an antibody comprising the HVR-H1 sequence of SEQ ID NO: 125, the HVR-H2 sequence of SEQ ID NO: 133, the HVR-H3 sequence of SEQ ID NO: 141, the HVR-L1 sequence of SEQ ID NO: 149, the HVR-L2 sequence of SEQ ID NO: 157, and the HVR-L3 sequence of SEQ ID NO: 165, and
13) an antibody comprising the HVR-H1 sequence of SEQ ID NO: 126, the HVR-H2 sequence of SEQ ID NO: 134, the HVR-H3 sequence of SEQ ID NO: 142, the HVR-L1 sequence of SEQ ID NO: 150, the HVR-L2 sequence of SEQ ID NO: 158, and the HVR-L3 sequence of SEQ ID NO: 166.

In some embodiments, an isolated antibody that inhibits the interaction between C1q and C1r2s2 complex of the present invention is an antibody having the antigen-binding activity which varies depending on an ion concentration. In some embodiments, an isolated antibody that inhibits the interaction between C1q and C1r2s2 complex of the present invention is an antibody having the C1s-binding activity which varies depending on an ion concentration. In some embodiments, an isolated antibody that inhibits the interaction between C1q and C1r2s2 complex of the present invention is an antibody having the C1r-binding activity which varies depending on an ion concentration.

In some embodiments, an isolated antibody that inhibits the interaction between C1q and C1r2s2 complex of the present invention is an antibody binding to the antigen with a higher affinity at neutral pH than at acidic pH. In some embodiments, an isolated antibody that inhibits the interaction between C1q and C1r2s2 complex of the present invention is an antibody binding to C1s with a higher affinity at neutral pH than at acidic pH. In some embodiments, an isolated antibody that inhibits the interaction between C1q and C1r2s2 complex of the present invention is an antibody binding to C1r with a higher affinity at neutral pH than at acidic pH.

In some embodiments, an isolated antibody that inhibits the interaction between C1q and C1r2s2 complex of the present invention is an antibody binding to the antigen with a higher affinity under a high calcium concentration condition than under a low calcium concentration condition. In some embodiments, an isolated antibody that inhibits the interaction between C1q and C1r2s2 complex of the present invention is an antibody binding to C1s with a higher affinity under a high calcium concentration condition than under a low calcium concentration condition. In some embodiments, an isolated antibody that inhibits the interaction between C1q and C1r2s2 complex of the present invention is an antibody binding to C1r with a higher affinity under a high calcium concentration condition than under a low calcium concentration condition.

In some embodiments, an isolated antibody that inhibits the interaction between C1q and C1r2s2 complex of the present invention is an antibody binding to the antigen with a higher affinity both at neutral pH and under a high calcium concentration condition than both at acidic pH and under a low calcium concentration. In some embodiments, an isolated antibody that inhibits the interaction between C1q and C1r2s2 complex of the present invention is an antibody binding to C1s with a higher affinity both at neutral pH and under a high calcium concentration condition than both at acidic pH and under a low calcium concentration. In some embodiments, an isolated antibody that inhibits the interaction between C1q and C1r2s2 complex of the present invention is an antibody binding to C1r with a higher affinity both at neutral pH and under a high calcium concentration condition than both at acidic pH and under a low calcium concentration.

In some embodiments, in an isolated antibody that inhibits the interaction between C1q and C1r2s2 complex of the present invention, the ratio of the KD value for its C1s-binding activity at acidic pH to the KD value for the C1s-binding activity at neutral pH (KD(acidic pH)/KD(neutral pH)) is 2 or more when measured at a high calcium concentration at both neutral and acidic pH. In some embodiments, an isolated antibody that inhibits the interaction between C1q and C1r2s2 complex of the present invention, the ratio of the KD value for its C1r-binding activity at acidic pH to the KD value for the C1r-binding activity at neutral pH (KD(acidic pH)/KD(neutral pH)) is 2 or more when measured at a high calcium concentration at both neutral and acidic pH.

In some embodiments, in an isolated antibody that inhibits the interaction between C1q and C1r2s2 complex of the present invention, the ratio of the KD value for its C1s-binding activity at acidic pH to the KD value for the C1s-binding activity at neutral pH (KD(acidic pH)/KD(neutral pH)) is 2 or more when measured at a low calcium concentration at both neutral and acidic pH, wherein the anti-C1s antibody binds to the dimeric state of C1s. In some embodiments, in an isolated antibody that inhibits the interaction between C1q and C1r2s2 complex of the present invention, the ratio of the KD value for its C1r-binding activity at acidic pH to the KD value for the C1r-binding activity at neutral pH (KD(acidic pH)/KD(neutral pH)) is 2 or more when measured at a low calcium concentration at both neutral and acidic pH, wherein the anti-C1s antibody binds to the dimeric state of C1r.

In some embodiments, in an isolated antibody that inhibits the interaction between C1q and C1r2s2 complex of the present invention, the ratio of the KD value for its C1s-binding activity at acidic pH to the KD value for the C1s-binding activity at neutral pH (KD(acidic pH)/KD(neutral pH)) is 2 or more when measured at a high calcium concentration at neutral pH and under low calcium concentration at acidic pH. In some embodiments, in an isolated antibody that inhibits the interaction between C1q and C1r2s2 complex of the present invention, the ratio of the KD value for its C1r-binding activity at acidic pH to the KD value for the C1r-binding activity at neutral pH (KD(acidic pH)/KD(neutral pH)) is 2 or more when measured at a high calcium concentration at neutral pH and under low calcium concentration at acidic pH.

In some embodiments, an anti-C1s antibody that inhibits the interaction between C1q and C1r2s2 complex of the present invention comprises a histidine residue at one or more of the following Kabat numbering system positions; Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In some embodiments, an anti-C1r antibody that inhibits the interaction between C1q and C1r2s2 complex of the present invention comprises a histidine residue at one or more of the following Kabat numbering system positions; Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In some embodiments, an anti-C1s antibody that inhibits the interaction between C1q and C1r2s2 complex of the present invention comprises at least one histidine which is substituted at one or more of the following Kabat numbering system positions; Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In some embodiments, an anti-C1r antibody that inhibits the interaction between C1q and C1r2s2 complex of the present invention comprises at least one histidine which is substituted at one or more of the following Kabat numbering system positions; Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In further embodiments, an anti-C1s antibody with pH dependency that inhibits the interaction between C1q and C1r2s2 complex of the present invention competes at neutral pH condition for binding to C1s with an antibody selected from the group consisting of 1)-5) below:
1) an antibody comprising the HVR-H1 sequence of SEQ ID NO: 32, the HVR-H2 sequence of SEQ ID NO: 33, the HVR-H3 sequence of SEQ ID NO: 34, the HVR-L1 sequence of SEQ ID NO: 35, the HVR-L2 sequence of SEQ ID NO: 36, and the HVR-L3 sequence of SEQ ID NO: 37,
2) an antibody comprising the HVR-H1 sequence of SEQ ID NO: 38, the HVR-H2 sequence of SEQ ID NO: 39, the HVR-H3 sequence of SEQ ID NO: 40, the HVR-L1 sequence of SEQ ID NO: 41, the HVR-L2 sequence of SEQ ID NO: 42, and the HVR-L3 sequence of SEQ ID NO: 43,
3) an antibody comprising the HVR-H1 sequence of SEQ ID NO: 44, the HVR-H2 sequence of SEQ ID NO: 45, the HVR-H3 sequence of SEQ ID NO: 46, the HVR-L1 sequence of SEQ ID NO: 47, the HVR-L2 sequence of SEQ ID NO: 48, and the HVR-L3 sequence of SEQ ID NO: 49,
4) an antibody comprising the HVR-H1 sequence of SEQ ID NO: 50, the HVR-H2 sequence of SEQ ID NO: 51, the HVR-H3 sequence of SEQ ID NO: 52, the HVR-L1 sequence of SEQ ID NO: 53, the HVR-L2 sequence of SEQ ID NO: 54, and the HVR-L3 sequence of SEQ ID NO: 55, and
5) an antibody comprising the HVR-H1 sequence of SEQ ID NO: 56, the HVR-H2 sequence of SEQ ID NO: 57, the HVR-H3 sequence of SEQ ID NO: 58, the HVR-L1 sequence of SEQ ID NO: 59, the HVR-L2 sequence of SEQ ID NO: 60, and the HVR-L3 sequence of SEQ ID NO: 61.

In further embodiments, an anti-C1r antibody with pH dependency that inhibits the interaction between C1q and C1r2s2 complex of the present invention competes at neutral pH condition for binding to C1r with an antibody selected from the group consisting of 6)-13) below:
6) an antibody comprising the HVR-H1 sequence of SEQ ID NO:119, the HVR-H2 sequence of SEQ ID NO: 127, the HVR-H3 sequence of SEQ ID NO: 135, the HVR-L1 sequence of SEQ ID NO: 143, the HVR-L2 sequence of SEQ ID NO: 151, and the HVR-L3 sequence of SEQ ID NO: 159,
7) an antibody comprising the HVR-H1 sequence of SEQ ID NO: 120, the HVR-H2 sequence of SEQ ID NO: 128, the HVR-H3 sequence of SEQ ID NO: 136, the HVR-L1 sequence of SEQ ID NO: 144, the HVR-L2 sequence of SEQ ID NO: 152, and the HVR-L3 sequence of SEQ ID NO: 160,
8) an antibody comprising the HVR-H1 sequence of SEQ ID NO: 121, the HVR-H2 sequence of SEQ ID NO: 129, the HVR-H3 sequence of SEQ ID NO: 137, the HVR-L1 sequence of SEQ ID NO: 145, the HVR-L2 sequence of SEQ ID NO: 153, and the HVR-L3 sequence of SEQ ID NO: 161,
9) an antibody comprising the HVR-H1 sequence of SEQ ID NO: 122, the HVR-H2 sequence of SEQ ID NO: 130, the HVR-H3 sequence of SEQ ID NO: 138, the HVR-L1 sequence of SEQ ID NO: 146, the HVR-L2 sequence of SEQ ID NO: 154, and the HVR-L3 sequence of SEQ ID NO: 162,
10) an antibody comprising the HVR-H1 sequence of SEQ ID NO: 123, the HVR-H2 sequence of SEQ ID NO: 131, the HVR-H3 sequence of SEQ ID NO: 139, the HVR-L1 sequence of SEQ ID NO: 147, the HVR-L2 sequence of SEQ ID NO: 155, and the HVR-L3 sequence of SEQ ID NO: 163,
11) an antibody comprising the HVR-H1 sequence of SEQ ID NO: 124, the HVR-H2 sequence of SEQ ID NO: 132, the HVR-H3 sequence of SEQ ID NO: 140, the HVR-L1 sequence of SEQ ID NO: 148, the HVR-L2 sequence of SEQ ID NO: 156, and the HVR-L3 sequence of SEQ ID NO: 164,
12) an antibody comprising the HVR-H1 sequence of SEQ ID NO: 125, the HVR-H2 sequence of SEQ ID NO: 133, the HVR-H3 sequence of SEQ ID NO: 141, the HVR-L1 sequence of SEQ ID NO: 149, the HVR-L2 sequence of SEQ ID NO: 157, and the HVR-L3 sequence of SEQ ID NO: 165, and
13) an antibody comprising the HVR-H1 sequence of SEQ ID NO: 126, the HVR-H2 sequence of SEQ ID NO: 134, the HVR-H3 sequence of SEQ ID NO: 142, the HVR-L1 sequence of SEQ ID NO: 150, the HVR-L2 sequence of SEQ ID NO: 158, and the HVR-L3 sequence of SEQ ID NO: 166.

In some embodiments, the present disclosure provides an isolated anti-C1s antibody that specifically binds to an epitope within a region encompassing the CUB1-EGF-CUB2 domain (also called interaction domain or CUB domain) consisting of CUB1, EGF, and CUB2 of complement component 1s (C1s), which is also called ‘CUB1-EGF-CUB2 domain of C1s’ in this description. In some embodiments, the antibody does not bind to the CCP1-CCP2-SP domain (also called catalytic domain, or CCP-SP domain) of C1s. In some embodiments, the epitope bound by an isolated anti-C1s antibody of the present disclosure is an epitope not located in beta domain of C1s. In some embodiments, the epitope bound by an isolated anti-C1s antibody of the present disclosure is an epitope located in alpha domain of C1s or gamma domain of C1s. In some embodiments, the epitope bound by an isolated anti-C1s antibody of the present disclosure is a linear epitope. In some embodiments, the epitope bound by an isolated anti-C1s antibody of the present disclosure is an epitope within amino acids 16-291 of the complement C1s protein, amino acids 16-172 of the complement C1s protein, amino acids 16-210 of the complement C1s protein, amino acids 16-111 of the complement C1s protein, amino acids 112-210 of the complement C1s protein, amino acids 131-172 of the complement C1s protein or amino acids 16-130 of the complement C1s protein. In some embodiments, the above-described epitope of C1s is an epitope of human C1s, or preferably an epitope of human C1s and an epitope of cynomolgus C1s.

In some embodiments, the present disclosure provides an isolated anti-C1r antibody that specifically binds to an epitope within a region encompassing the CUB1-EGF-CUB2 domain consisting of CUB1, EGF, and CUB2 of complement component 1r (C1r), which is also called ‘CUB1-EGF-CUB2 domain of C1r’ in this description. In some embodiments, the antibody does not bind to the CCP1-CCP2-SP domain (also called catalytic domain) of C1r. In some cases, the epitope bound by an isolated anti-C1r antibody of the present disclosure is a linear epitope or conformational epitope. In some embodiments, the above-described epitope of C1r is an epitope of human C1r, or preferably an epitope of human C1r and an epitope of cynomolgus C1r.

In some embodiments, an isolated anti-C1s antibody of the present invention comprises (a) HVR-H1 comprising the amino acid sequence of SEQ ID NO: 32, 38, 44, 50, or 56, (b) HVR-H2 comprising the amino acid sequence of SEQ ID NO: 33, 39, 45, 51, or 57 and (c) HVR-H3 comprising the amino acid sequence of SEQ ID NO: 34, 40, 46, 52, or 58, wherein the antibody comprises human-derived or primate-derived framework regions. In some embodiments, an isolated anti-C1s antibody of the present invention comprises (a) HVR-L1 comprising the amino acid sequence of SEQ ID NO: 35, 41, 47, 53, or 59; (b) HVR-L2 comprising the amino acid sequence of SEQ ID NO: 36, 42, 48, 54, or 60; and (c) HVR-L3 comprising the amino acid sequence of SEQ ID NO: 37, 43, 49, 55, or 61, wherein the antibody comprises human-derived or primate-derived framework regions.

In some embodiments, an isolated anti-C1r antibody of the present invention comprises (a) HVR-H1 comprising the amino acid sequence of SEQ ID NO: 119, 120, 121, 122, 123, 124, 125, or 126, (b) HVR-H2 comprising the amino acid sequence of SEQ ID NO: 127, 128, 129, 130, 131, 132, 133, or 134 and (c) HVR-H3 comprising the amino acid sequence of SEQ ID NO: 135, 136, 137, 138, 139, 140, 141, or 142, wherein the antibody comprises human-derived or primate-derived framework regions. In some embodiments, an isolated anti-C1r antibody of the present invention comprises (a) HVR-L1 comprising the amino acid sequence of SEQ ID NO: 143, 144, 145, 146, 147, 148, 149, or 150; (b) HVR-L2 comprising the amino acid sequence of SEQ ID NO: 151, 152, 153, 154, 155, 156, 157, or 158; and (c) HVR-L3 comprising the amino acid sequence of SEQ ID NO: 159, 160, 161, 162, 163, 164, 165, or 166, wherein the antibody comprises human-derived or primate-derived framework regions.

In some embodiments, anti-C1s antibody of the present invention comprises (a) a VH sequence having at least 95% sequence identity to the amino acid sequence of SEQ ID NO: 19, 20, 21, 23, or 24; (b) a VL sequence having at least 95% sequence identity to the amino acid sequence of SEQ ID NO: 26, 27, 28, 30, or 31; or (c) a VH sequence of (a) and a VL sequence of (b). In some embodiments, an anti-C1s antibody of the present invention comprises a VH sequence of SEQ ID NO: 19, 20, 21, 23, or 24. In some embodiments, an anti-C1s antibody of the present invention comprises a VL sequence of SEQ ID NO: 26, 27, 28, 30, or 31. In further embodiments, an anti-C1s antibody of the present invention comprises a VH sequence of SEQ ID NO: 19, 20, 21, 23, or 24 and a VL sequence of SEQ ID NO: 26, 27, 28, 30, or 31. In further embodiments, an anti-C1s antibody of the present invention comprises a VH sequence of SEQ ID NO: 19 and a VL sequence of SEQ ID NO: 26. In further embodiments, an anti-C1s antibody of the present invention comprises a VH sequence of SEQ ID NO: 20 and a VL sequence of SEQ ID NO: 27. In further embodiments, an anti-C1s antibody of the present invention comprises a VH sequence of SEQ ID NO: 21 and a VL sequence of SEQ ID NO: 28. In further embodiments, an anti-C1s antibody of the present invention comprises a VH sequence of SEQ ID NO: 23 and a VL sequence of SEQ ID NO: 30. In further embodiments, an anti-C1s antibody of the present invention comprises a VH sequence of SEQ ID NO: 24 and a VL sequence of SEQ ID NO: 31.

In some embodiments, anti-C1r antibody of the present invention comprises (a) a VH sequence having at least 95% sequence identity to the amino acid sequence of SEQ ID NO: 103, 104, 105, 106, 107, 108, 109, or 110; (b) a VL sequence having at least 95% sequence identity to the amino acid sequence of SEQ ID NO: 111, 112, 113, 114, 115, 116, 117, or 118; or (c) a VH sequence of (a) and a VL sequence of (b). In some embodiments, an anti-C1r antibody of the present invention comprises a VH sequence of SEQ ID NO: 103, 104, 105, 106, 107, 108, 109, or 110. In some embodiments, an anti-C1r antibody of the present invention comprises a VL sequence of SEQ ID NO: 111, 112, 113, 114, 115, 116, 117, or 118. In further embodiments, an anti-C1r antibody of the present invention comprises a VH sequence of SEQ ID NO: 103, 104, 105, 106, 107, 108, 109, or 110 and a VL sequence of SEQ ID NO: 111, 112, 113, 114, 115, 116, 117, or 118. In further embodiments, an anti-C1r antibody of the present invention comprises a VH sequence of SEQ ID NO: 103 and a VL sequence of SEQ ID NO: 111. In further embodiments, an anti-C1r antibody of the present invention comprises a VH sequence of SEQ ID NO: 104 and a VL sequence of SEQ ID NO: 112. In further embodiments, an anti-C1r antibody of the present invention comprises a VH sequence of SEQ ID NO: 105 and a VL sequence of SEQ ID NO: 113. In further embodiments, an anti-C1r antibody of the present invention comprises a VH sequence of SEQ ID NO: 106 and a VL sequence of SEQ ID NO: 114. In further embodiments, an anti-C1r antibody of the present invention comprises a VH sequence of SEQ ID NO: 107 and a VL sequence of SEQ ID NO: 115. In further embodiments, an anti-C1r antibody of the present invention comprises a VH sequence of SEQ ID NO: 108 and a VL sequence of SEQ ID NO: 116. In further embodiments, an anti-C1r antibody of the present invention comprises a VH sequence of SEQ ID NO: 109 and a VL sequence of SEQ ID NO: 117. In further embodiments, an anti-C1r antibody of the present invention comprises a VH sequence of SEQ ID NO: 110 and a VL sequence of SEQ ID NO: 118.

In some embodiments, an anti-C1s antibody that inhibits the interaction between C1q and C1r2s2 complex of the present invention is a monoclonal antibody. In some embodiments, an anti-C1s antibody that inhibits the interaction between C1q and C1r2s2 complex of the present invention is a human, humanized, or chimeric antibody. In further embodiments, an anti-C1s antibody that inhibits the interaction between C1q and C1r2s2 complex of the present invention is a full length IgG1, IgG2, IgG3 or IgG4 antibody. In further embodiments, an anti-C1s antibody that inhibits the interaction between C1q and C1r2s2 complex of the present invention is an antibody fragment that binds to C1s. In some specific embodiments, an anti-C1s antibody that inhibits the interaction between C1q and C1r2s2 complex of the present invention is a human IgG1 or humanized IgG1.

In some embodiments, an anti-C1r antibody that inhibits the interaction between C1q and C1r2s2 complex of the present invention is a monoclonal antibody. In some embodiments, an anti-C1r antibody that inhibits the interaction between C1q and C1r2s2 complex of the present invention is a human, humanized, or chimeric antibody. In further embodiments, an anti-C1r antibody that inhibits the interaction between C1q and C1r2s2 complex of the present invention is a full length IgG1, IgG2, IgG3 or IgG4 antibody. In further embodiments, an anti-C1r antibody that inhibits the interaction between C1q and C1r2s2 complex of the present invention is an antibody fragment that binds to C1r. In some specific embodiments, an anti-C1r antibody that inhibits the interaction between C1q and C1r2s2 complex of the present invention is a human IgG1 or humanized IgG1.

In some embodiments, an isolated antibody that inhibits the interaction between C1q and C1r2s2 complex of the present invention is an antibody comprising an Fc region that has at least one amino acid modification in the region so as to enhance the reduction of plasma antigen concentration and/or improve pharmacokinetics of the antibody.

In some embodiments, an isolated antibody that inhibits the interaction between C1q and C1r2s2 complex of the present invention has a human Fc region that has a binding activity selected from the following group consisting of:
a) a binding activity to an activating Fc gamma receptor is stronger than the binding activity of an Fc region of the native human IgG1,
b) a binding activity to an inhibitory Fc gamma receptor is stronger than to an activating Fc gamma receptor, and
c) a binding activity to an FcRn at neutral pH is stronger than the binding activity of an Fc region of the native human IgG1.

In some embodiments, an isolated antibody that inhibits the interaction between C1q and C1r2s2 complex of the present invention binds to at least human C1s or preferably to both cynomolgus C1s and human C1s. In some embodiments, an isolated antibody that inhibits the interaction between C1q and C1r2s2 complex of the present invention binds to at least human C1r or preferably to both cynomolgus C1r and human C1r.

The invention also provides isolated nucleic acids encoding an anti-C1s antibody of the present invention. The invention also provides isolated nucleic acids encoding an anti-C1r antibody of the present invention. The invention also provides host cells comprising a nucleic acid of the present invention. The invention also provides a method of producing an antibody comprising culturing a host cell of the present invention so that the antibody is produced.

The invention also provides a pharmaceutical formulation comprising the antibody of the present invention and a pharmaceutically acceptable carrier.

Anti-C1s antibodies of the present invention may be for use as a medicament. Anti-C1s antibodies of the present invention may be for use in treating or preventing a complement-mediated disease or disorder. Anti-C1s antibodies of the present invention may be for use in enhancing the clearance of (or removing) C1s from plasma. Anti-C1s antibodies of the present invention may be for use in enhancing the clearance of (or removing) C1r2s2 from plasma. Anti-C1s antibodies of the present invention may be for use in enhancing the clearance of (or removing) C1r2s2 from plasma but not C1q from plasma. In some cases, the antibody inhibits a component of the classical complement pathway; in some cases, the classical complement pathway component is C1s.

Anti-C1r antibodies of the present invention may be for use as a medicament. Anti-C1r antibodies of the present invention may be for use in treating or preventing a complement-mediated disease or disorder. Anti-C1r antibodies of the present invention may be for use in enhancing the clearance of (or removing) C1r from plasma. Anti-C1r antibodies of the present invention may be for use in enhancing the clearance of (or removing) C1r2s2 from plasma. Anti-C1r antibodies of the present invention may be for use in enhancing the clearance of (or removing) C1r2s2 from plasma but not C1q from plasma. In some cases, the antibody inhibits a component of the classical complement pathway; in some cases, the classical complement pathway component is C1r.

Anti-C1s antibodies of the present invention may be used in the manufacture of a medicament. In some embodiments, the medicament is for treatment or prevention of a complement-mediated disease or disorder. In some embodiments, the medicament is for enhancing the clearance of (or removing) C1s from plasma. In some embodiments, the medicament is for enhancing the clearance of (or removing) C1r2s2 from plasma. In some embodiments, the medicament is for enhancing the clearance of (or removing) C1r2s2 from plasma but not C1q from plasma. In this context, the level of the enhancement of C1q clearance from plasma is not necessarily null (zero). That is, the level of the enhancement of C1q clearance from plasma may be zero, or may not be zero but near zero, or may be non-significant or very low enough to be technically neglected by those skilled in the art. In some cases, the medicament inhibits a component of the classical complement pathway; in some cases, the classical complement pathway component is C1s.

The enhancement of C1s/C1q clearance (CL) can be measured, for example, as follows.

The total concentrations of human C1s and C1q in mouse plasma are measured by LC/ESI-MS/MS. The calibration standards are prepared by mixing and diluting human C1s and C1q in defined amounts in mouse plasma, resulting in human C1s concentrations of 0.477, 0.954, 1.91, 3.82, 7.64, 15.3, 30.5 micrograms (micro g)/mL and human C1q concentrations of 0.977, 1.95, 3.91, 7.81, 15.6, 31.3 and 62.5 micro g/mL, respectively. A 2 micro L of the calibration standards and plasma samples is mixed with 25 micro L of 6.8 mol/L Urea, 9.1 mmol/L dithiothreitol and 0.4 micro g/mL lysozyme (chicken egg white) in 50 mmol/L ammonium bicarbonate and incubated for 45 min at 56 degrees C. Then, 2 micro L of 500 mmol/L iodoacetamide is added and incubated for 30 min at 37 degrees C. in the dark. Next, 160 micro L of 0.5 micro g/mL sequencing grade modified trypsin (Promega) in 50 mmol/L ammonium bicarbonate is added and incubated at 37 degrees C. overnight. Finally, 5 micro L of 10% trifluoroacetic acid is added to deactivate any residual trypsin. A 40 micro L of digestion samples are subjected to analysis by LC/ESI-MS/MS. LC/ESI-MS/MS is performed using Xevo TQ-S triple quadrupole instrument (Waters) equipped with 2D I-class UPLC (Waters). Human C1s specific peptide LLEVPEGR and human C1q specific peptide IAFSATR are monitored by the selected reaction monitoring (SRM). SRM transition is [M+2H]2+ (m/z 456.8) to y6 ion (m/z 686.3) for human C1s, and [M+2H]2+ (m/z 383.2) to y5 ion (m/z 581.3) for human C1q. Calibration curve is constructed by the weighted (1/×2) linear regression using the peak area plotted against the concentrations. The concentration in mouse plasma is calculated from the calibration curve using the analytical software Masslynx Ver.4.1 (Waters).

Pharmacokinetics for total hC1s and hC1q after administration of anti-C1s antibodies in mice is evaluated as follows.

The in vivo pharmacokinetics of hC1s, hC1q and anti-C1s antibodies is assessed after administering antigen alone (hC1q, recombinant C1r2s2, mixture of hC1q and rC1r2s2) or with anti-C1s antibody to mice (CB17/Icr-Prkdcscid/Cr1Crl: Charles River Japan). Three mice are allocated to each dosing group.

Firstly, hC1q solution (0.84 mg/mL), rC1r2s2 (0.47 mg/mL) or a solution of mixture containing hC1q and rC1r2s2 (0.84 and 0.47 mg/mL, respectively) is injected at a dose of 10 mL/kg to mice intravenously. After dosing of antigen solution, anti-C1s antibody solution (2.5 mg/mL) is immediately administered to the same individual in the same way.

The dose setting of C1q and rC1r2s2 is designed to be physiological concentration in human plasma just after administration. Dosage of anti-C1s antibody is adjusted to be excess concentration over both antigens during the study, and thus almost all hC1s is assumed to be bound form in circulation.

Blood is collected at 5, 30 minutes, 2, 7 hours, 3, 7, 14, 21 and 28 days after injection. The blood is centrifuged immediately to separate the plasma samples. Plasma concentrations of hC1s and hC1q are measured at each sampling points by LC/ESI-MS/MS. PK parameters of hC1s and hC1q are estimated by non-compartmental analysis (Phoenix WinNonlin version 8.0, Certara).

Mice are administered with an antibody with (i) an Fc containing mutations to reduce both C1q and Fc gamma receptor binding, or (ii) an Fc containing mutation to reduce C1q binding while retaining Fc gamma receptor binding. For example, in the present invention, Fc of “SG136” contains mutations to reduce both C1q and Fc gamma receptor binding, while Fc of “SG1148” contains mutation to reduce C1q binding while retaining Fc gamma receptor binding.

The above mice PK study is conducted for a test antibody (e.g., CCP1-CCP2-SP or CUB1-EGF-CUB2 binder), and PK parameters of hC1q and hC1s are calculated. Then, C1s CL ratio (SG1148/SG136) of the binder or C1q CL ratio (SG1148/SG136) of the binder can be evaluated. In some embodiments, the C1q CL ratio of the antibody of the present invention is 1.8 or less, 1.7 or less, 1.6 or less, 1.5 or less, 1.4 or less, 1.3 or less, 1.2 or less, 1.1 or less, 1.0 or less.

Anti-C1r antibodies of the present invention may be used in the manufacture of a medicament. In some embodiments, the medicament is for treatment or prevention of a complement-mediated disease or disorder. In some embodiments, the medicament is for enhancing the clearance of (or removing) C1r from plasma. In some embodiments, the medicament is for enhancing the clearance of (or removing) C1r2s2 from plasma. In some embodiments, the medicament is for enhancing the clearance of (or removing) C1r2s2 from plasma but not C1q from plasma. In some cases, the medicament inhibits a component of the classical complement pathway; in some cases, the classical complement pathway component is C1r.

The invention also provides a method of treating or preventing an individual having a complement-mediated disease or disorder. In some embodiments, the method comprises administering to the individual an effective amount of an anti-C1s antibody of the present invention. The invention also provides a method of enhancing the clearance of (or removing) C1s from plasma in an individual. In some embodiments, the method comprises administering to the individual an effective amount of an anti-C1s antibody of the present invention to enhance the clearance of (or remove) C1s from plasma. The invention also provides a method of enhancing the clearance of (or removing) C1r2s2 from plasma in an individual. The invention also provides a method of enhancing the clearance of (or removing) C1r2s2 from plasma not but C1q from plasma in an individual. In some embodiments, the method comprises administering to the individual an effective amount of an anti-C1s antibody of the present invention to enhance the clearance of (or remove) C1r2s2 from plasma. In some embodiments, the method comprises administering to the individual an effective amount of an anti-C1s antibody of the present invention to enhance the clearance of (or remove) C1r2s2 from plasma not but C1q from plasma. In some cases, the antibody inhibits a component of the classical complement pathway; in some cases, the classical complement pathway component is C1s.

The invention also provides a method of treating or preventing an individual having a complement-mediated disease or disorder. In some embodiments, the method comprises administering to the individual an effective amount of an anti-C1r antibody of the present invention. The invention also provides a method of enhancing the clearance of (or removing) C1r from plasma in an individual. In some embodiments, the method comprises administering to the individual an effective amount of an anti-C1r antibody of the present invention to enhance the clearance of (or remove) C1r from plasma. The invention also provides a method of enhancing the clearance of (or removing) C1r2s2 from plasma in an individual. The invention also provides a method of enhancing the clearance of (or removing) C1r2s2 from plasma not but C1q from plasma in an individual. In some embodiments, the method comprises administering to the individual an effective amount of an anti-C1r antibody of the present invention to enhance the clearance of (or remove) C1r2s2 from plasma. In some embodiments, the method comprises administering to the individual an effective amount of an anti-C1r antibody of the present invention to enhance the clearance of (or remove) C1r2s2 from plasma not but C1q from plasma. In some cases, the antibody inhibits a component of the classical complement pathway; in some cases, the classical complement pathway component is C1r.

More specifically, the present invention provides the following:
[1] An isolated antibody that inhibits the interaction between C1q and C1r2s2 complex, wherein the antibody has a displacement function such that the antibody binds to C1qrs complex and promotes dissociation of C1q from C1qrs complex.
[2] The antibody of [1], wherein the antibody binds to C1qrs complex on a Biacore chip and promotes dissociation of C1q from C1qrs complex, wherein a value of response unit (RU) in presence of the antibody is lower than a value of response unit (RU) in the absence of the antibody as determined by a Biacore assay when a sufficient time passed.
[3] The antibody of [2], wherein the time point of crossover in the Biacore assay is within 60 s, 100 s, 150 s, 200 s, 500 s, 700 s, or 1000 s after the time point of the start of antibody injection as determined by the Biacore assay using the following conditions: the capture levels of C1r2s2 complex and C1q are at 200 resonance unit (RU) and 200 resonance unit (RU), respectively, and the antibody as an analyte is injected at 500 nM at 10 microliter/min.
[4] The antibody of [2], wherein almost all of C1q are dissociated from C1qrs complex within 100 s, 300 s, 500 s, 700 s, 1000 s, 1500 s or 2000 s after the time point of the start of antibody injection as determined by the Biacore assay using the following conditions: the capture levels of C1r2s2 complex and C1q are at 200 resonance unit (RU) and 200 resonance unit (RU), respectively, and the antibody as an analyte is injected at 500 nM at 10 microliter/min.
[5] An isolated antibody that inhibits the interaction between C1q and C1r2s2 complex, wherein the antibody has a neutralizing activity for human serum complement of at least 70% in an RBC assay.
[6] The antibody of any one of [1] to [5], wherein the antibody is an antibody that specifically binds to C1s or an antibody that specifically binds to C1r.
[7] An isolated antibody that inhibits the interaction between C1q and C1r2s2 complex,
   wherein the antibody specifically binds to an epitope within a CUB1-EGF-CUB2 domain of C1s, and competes for binding to the epitope with an antibody selected from the group consisting of 1)-5) below:
      1) an antibody comprising the HVR-H1 sequence of SEQ ID NO: 32, the HVR-H2 sequence of SEQ ID NO: 33, the HVR-H3 sequence of SEQ ID NO: 34, the HVR-L1 sequence of SEQ ID NO: 35, the HVR-L2 sequence of SEQ ID NO: 36, and the HVR-L3 sequence of SEQ ID NO: 37,
      2) an antibody comprising the HVR-H1 sequence of SEQ ID NO: 38, the HVR-H2 sequence of SEQ ID NO: 39, the HVR-H3 sequence of SEQ ID NO: 40, the HVR-L1 sequence of SEQ ID NO: 41, the HVR-L2 sequence of SEQ ID NO: 42, and the HVR-L3 sequence of SEQ ID NO: 43,
      3) an antibody comprising the HVR-H1 sequence of SEQ ID NO: 44, the HVR-H2 sequence of SEQ ID NO: 45, the HVR-H3 sequence of SEQ ID NO: 46, the HVR-L1 sequence of SEQ ID NO: 47, the HVR-L2 sequence of SEQ ID NO: 48, and the HVR-L3 sequence of SEQ ID NO: 49,
      4) an antibody comprising the HVR-H1 sequence of SEQ ID NO: 50, the HVR-H2 sequence of SEQ ID NO: 51, the HVR-H3 sequence of SEQ ID NO: 52, the HVR-L1 sequence of SEQ ID NO: 53, the HVR-L2 sequence of SEQ ID NO: 54, and the HVR-L3 sequence of SEQ ID NO: 55, and
      5) an antibody comprising the HVR-H1 sequence of SEQ ID NO: 56, the HVR-H2 sequence of SEQ ID NO: 57, the HVR-H3 sequence of SEQ ID NO: 58, the HVR-L1 sequence of SEQ ID NO: 59, the HVR-L2 sequence of SEQ ID NO: 60, and the HVR-L3 sequence of SEQ ID NO: 61, or wherein the antibody specifically binds to an epitope within a CUB1-EGF-CUB2 domain of C1r, and competes for binding to the epitope with an antibody selected from the group consisting of 6)-13) below:
      6) an antibody comprising the HVR-H1 sequence of SEQ ID NO:119, the HVR-H2 sequence of SEQ ID NO: 127, the HVR-H3 sequence of SEQ ID NO: 135, the HVR-L1 sequence of SEQ ID NO: 143, the HVR-L2 sequence of SEQ ID NO: 151, and the HVR-L3 sequence of SEQ ID NO: 159,
      7) an antibody comprising the HVR-H1 sequence of SEQ ID NO: 120, the HVR-H2 sequence of SEQ ID NO: 128, the HVR-H3 sequence of SEQ ID NO: 136, the HVR-L1 sequence of SEQ ID NO: 144, the HVR-L2 sequence of SEQ ID NO: 152, and the HVR-L3 sequence of SEQ ID NO: 160,
      8) an antibody comprising the HVR-H1 sequence of SEQ ID NO: 121, the HVR-H2 sequence of SEQ ID NO: 129, the HVR-H3 sequence of SEQ ID NO: 137, the HVR-L1 sequence of SEQ ID NO: 145, the HVR-L2 sequence of SEQ ID NO: 153, and the HVR-L3 sequence of SEQ ID NO: 161,
      9) an antibody comprising the HVR-H1 sequence of SEQ ID NO: 122, the HVR-H2 sequence of SEQ ID NO: 130, the HVR-H3 sequence of SEQ ID NO: 138, the HVR-L1 sequence of SEQ ID NO: 146, the HVR-L2 sequence of SEQ ID NO: 154, and the HVR-L3 sequence of SEQ ID NO: 162,
      10) an antibody comprising the HVR-H1 sequence of SEQ ID NO: 123, the HVR-H2 sequence of SEQ ID NO: 131, the HVR-H3 sequence of SEQ ID NO: 139, the HVR-L1 sequence of SEQ ID NO: 147, the HVR-L2 sequence of SEQ ID NO: 155, and the HVR-L3 sequence of SEQ ID NO: 163,
      11) an antibody comprising the HVR-H1 sequence of SEQ ID NO: 124, the HVR-H2 sequence of SEQ ID NO: 132, the HVR-H3 sequence of SEQ ID NO: 140, the HVR-L1 sequence of SEQ ID NO: 148, the HVR-L2 sequence of SEQ ID NO: 156, and the HVR-L3 sequence of SEQ ID NO: 164,
      12) an antibody comprising the HVR-H1 sequence of SEQ ID NO: 125, the HVR-H2 sequence of SEQ ID NO: 133, the HVR-H3 sequence of SEQ ID NO: 141, the HVR-L1 sequence of SEQ ID NO: 149, the HVR-L2 sequence of SEQ ID NO: 157, and the HVR-L3 sequence of SEQ ID NO: 165, and
      13) an antibody comprising the HVR-H1 sequence of SEQ ID NO: 126, the HVR-H2 sequence of SEQ ID NO: 134, the HVR-H3 sequence of SEQ ID NO: 142, the HVR-L1 sequence of SEQ ID NO: 150, the HVR-L2 sequence of SEQ ID NO: 158, and the HVR-L3 sequence of SEQ ID NO: 166.
[8] An isolated antibody that inhibits the interaction between C1q and C1r2s2 complex, wherein the antigen-binding activity of the antibody is lower at pH 5.8 than at pH 7.4.
[9] The antibody of any one of [1] to [8], wherein the antibody specifically binds to an epitope within a CUB1-EGF-CUB2 domain of C1s or C1r, wherein the antigen-binding activity of the antibody is lower at pH 5.8 than at pH 7.4.
[10] The antibody of [9], wherein the antibody binds to C1s or C1r with a lower affinity at acidic pH than at neutral pH as described in (i) or (ii) below:
   (i) when measured at a high calcium concentration at both neutral and acidic pH, the ratio of the KD value for C1s-binding activity at acidic pH to the KD value for C1s-binding activity at neutral pH (KD(acidic pH)/KD(neutral pH)) is 2 or more,
   (ii) when measured at a high calcium concentration at neutral pH and at a low calcium concentration at acidic pH, the ratio of the KD value for C1s-binding activity at acidic pH to the KD value for C1s-binding activity at neutral pH (KD(acidic pH)/KD(neutral pH)) is 2 or more.
[11] The antibody of any one of [8] to [10], wherein the antibody comprises an Fc region that has at least one amino acid modification in the region so as to enhance the reduction of plasma antigen concentration and/or improve pharmacokinetics of the antibody.
[12] The antibody of [11], wherein the Fc region is a human Fc region that has a binding activity selected from the following group consisting of:
   a) a binding activity to an activating Fc gamma receptor is stronger than the binding activity of an Fc region of the native human IgG1,
   b) a binding activity to an inhibitory Fc gamma receptor is stronger than to an activating Fc gamma receptor, and
   c) a binding activity to an FcRn at neutral pH is stronger than the binding activity of an Fc region of the native human IgG1.
[13] The antibody of any one of [1] to [12], wherein the antibody binds to both cynomolgus C1s and human C1s, or to both cynomolgus C1r and human C1r.
[14] A pharmaceutical formulation comprising the antibody of any one of [1] to [13] and a pharmaceutically acceptable carrier.
[15] A method of treating an individual having a complement-mediated disease or disorder comprising administering to the individual an effective amount of the antibody of any one of [1] to [13].

### BRIEF DESCRIPTION OF DRAWINGS

FIG. 1A illustrates the binding specificity of antibodies to the CUB1-EGF-CUB2 domain of C1s protein. BIACORE® sensorgrams of anti-C1s antibodies against recombinant human C1s CCP1-CCP2-SP-His protein.

FIG. 1B illustrates the binding specificity of antibodies to the CUB1-EGF-CUB2 domain of C1s protein. BIACORE® sensorgrams of anti-C1s antibodies against native proenzyme human C1s protein.

FIG. 2A illustrates antibody-mediated displacement of native human C1q from recombinant human C1r2s2 Flag/His tetramer immobilized onto the BIACORE® sensor surface. The displacement of native human C1q by the antibody is described by overwriting of 3 sensorgrams. Sensorgram 1 (small dotted line) describes the stable capture of C1qrs onto the sensor surface. Sensorgram 2 (large dotted line) describes the binding of antibody to C1qrs and displacement of C1q from C1r2s2. Sensorgram 3 (solid line) describes the baseline when only antibody is bound to C1r2s2 in the absence of any C1q. For comparison of these sensorgrams, the RU at time 0 is normalized (i.e., set to be the same) in FIG. 2A.

FIG. 2B illustrates antibody-mediated displacement of native human C1q from recombinant human C1r2s2 Flag/His tetramer immobilized onto the BIACORE® sensor surface. The displacement of native human C1q by the antibody is described by overwriting of 3 sensorgrams. Sensorgram 1 (small dotted line) describes the stable capture of C1qrs onto the sensor surface. Sensorgram 2 (large dotted line) describes the binding of antibody to C1qrs and displacement of C1q from C1r2s2. Sensorgram 3 (solid line) describes the baseline when only antibody is bound to C1r2s2 in the absence of any C1q. For comparison of these sensorgrams, the RU at time 0 is normalized (i.e., set to be the same) in FIG. 2B.

FIG. 2C illustrates antibody-mediated displacement of native human C1q from recombinant human C1r2s2 Flag/His tetramer immobilized onto the BIACORE® sensor surface. The displacement of native human C1q by the antibody is described by overwriting of 3 sensorgrams. Sensorgram 1 (small dotted line) describes the stable capture of C1qrs onto the sensor surface. Sensorgram 2 (large dotted line) describes the binding of antibody to C1qrs and displacement of C1q from C1r2s2. For comparison of these sensorgrams, the RU at the Ab injection is normalized (i.e., set to be the same) in FIG. 2C.

FIG. 2D illustrates antibody-mediated displacement of native human C1q from recombinant human C1r2s2 Flag/His tetramer immobilized onto the BIACORE® sensor surface. The displacement of native human C1q by the antibody is described by overwriting of 3 sensorgrams. Sensorgram 1 (small dotted line) describes the stable capture of C1qrs onto the sensor surface. Sensorgram 2 (large dotted line) describes the binding of antibody to C1qrs and displacement of C1q from C1r2s2. For comparison of these sensorgrams, the RU at the Ab injection is normalized (i.e., set to be the same) in FIG. 2D.

FIG. 3 illustrates antibody-mediated displacement of recombinant human C1r2s2 Flag/His tetramer from biotinylated native human C1q that has been immobilized onto the BIACORE® sensor surface. Recombinant human C1r2s2 Flag/His tetramer was flowed to bind to immobilized native human C1q, followed by the flow of either buffer alone to monitor the dissociation rate of C1r2s2 (solid line), or flow of antibody to dissociate C1r2s2 (dotted line).

FIG. 4 illustrates antibody-mediated blocking of native human C1q binding to recombinant human C1r2s2 Flag/His tetramer. The antibodies with C1q blocking function competed with C1q for binding to C1r2s2.

FIG. 5 illustrates the neutralization of human serum complement activity.

FIG. 6 illustrates the competitive epitope binning results of antibodies that bind to the CUB1-EGF-CUB2 domain of C1s.

FIG. 7 illustrates the pharmacokinetics of human C1s and human C1q after administration of anti-C1s antibodies in mice.

FIG. 8 illustrates the time dependent neutralization of human serum complement activity by anti-C1s antibodies.

FIG. 9 illustrates the antibody binding to native human proenzyme C1s in reducing and non-reducing western blotting analysis.

FIG. 10 illustrates the antibody binding to truncated C1s proteins in reducing western blot.

FIG. 11A illustrates the binding specificity of antibodies to the CUB1-EGF-CUB2 domain of C1r protein. BIACORE® sensorgrams of anti-C1r antibodies against recombinant human C1r CCP1-CCP2-SP-FLAG protein.

FIG. 11B illustrates the binding specificity of antibodies to the CUB1-EGF-CUB2 domain of C1r protein. BIACORE® sensorgrams of anti-C1r antibodies against native human C1r enzyme.

FIG. 12A illustrates antibody-mediated displacement of native human C1q from recombinant human C1r2s2 Flag/His tetramer captured onto the BIACORE® sensor surface. The displacement of native human C1q by the antibody is described by overlaying of 3 sensorgrams. Sensorgram 1 (small dotted line) describes the stable capture of C1qrs onto the sensor surface. Sensorgram 2 (large dotted line) describes the binding of antibody to C1qrs and displacement of C1q from C1r2s2. Sensorgram 3 (solid line) describes the baseline when only antibody is bound to C1r2s2 in the absence of any C1q. For comparison of these sensorgrams, the RU at time 0 is normalized (i.e., set to be the same) in FIG. 12A.

FIG. 12B illustrates antibody-mediated displacement of native human C1q from recombinant human C1r2s2 Flag/His tetramer captured onto the BIACORE® sensor surface. The displacement of native human C1q by the antibody is described by overlaying of 3 sensorgrams. Sensorgram 1 (small dotted line) describes the stable capture of C1qrs onto the sensor surface. Sensorgram 2 (large dotted line) describes the binding of antibody to C1qrs and displacement of C1q from C1r2s2. Sensorgram 3 (solid line) describes the baseline when only antibody is bound to C1r2s2 in the absence of any C1q. For comparison of these sensorgrams, the RU at time 0 is normalized (i.e., set to be the same) in FIG. 12B.

FIG. 12C illustrates antibody-mediated displacement of native human C1q from recombinant human C1r2s2 Flag/His tetramer captured onto the BIACORE® sensor surface. The displacement of native human C1q by the antibody is described by overlaying of 2 sensorgrams. Sensorgram 1 (solid line) describes the stable capture of C1qrs onto the sensor surface. Sensorgram 2 (dotted line) describes the binding of antibody to C1qrs and displacement of C1q from C1r2s2. For comparison of these sensorgrams, the RU at the Ab injection is normalized (i.e., set to be the same) in FIG. 12C.

FIG. 12D illustrates antibody-mediated displacement of native human C1q from recombinant human C1r2s2 Flag/His tetramer captured onto the BIACORE® sensor surface. The displacement of native human C1q by the antibody is described by overlaying of 2 sensorgrams. Sensorgram 1 (solid line) describes the stable capture of C1qrs onto the sensor surface. Sensorgram 2 (dotted line) describes the binding of antibody to C1qrs and displacement of C1q from C1r2s2. For comparison of these sensorgrams, the RU at the Ab injection is normalized (i.e., set to be the same) in FIG. 12D.

FIG. 13 illustrates the neutralization of human serum complement activity.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### I. DEFINITIONS

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

For purposes of interpreting this specification, the following definitions will apply and whenever appropriate, terms used in the singular will also include the plural and vice versa. It is to be understood that the terminology used herein is for the purpose of describing particular embodiments only, and is not intended to be limiting. In the event that any definition set forth below conflicts with any document incorporated herein by reference, the definition set forth below shall control.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd or KD). Affinity can be measured by common methods known in the art, including those described herein. Specific illustrative and exemplary embodiments for measuring binding affinity are described in the following. “Affinity”, “binding affinity”, “binding ability”, and “binding activity” may be used interchangeably. The term “binding activity” refers to the strength of the sum total of noncovalent interactions between a single or more binding sites of a molecule (e.g., an antibody) and its binding partner (e.g., an antigen). Herein, binding activity is not strictly limited to an activity which reflects a 1:1 interaction between members of a binding pair (e.g., antibody and antigen). When members of a binding pair can bind to each other in the manner of both monovalent and multivalent binding, binding activity is the strength of the sum total of these bindings. The binding activity of a molecule X for its partner Y can generally be represented by the dissociation constant (KD). Alternatively, the association and dissociation rates (Kon and Koff) may be used for the assessment of binding. Binding activity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-C1s antibody” and “an antibody that binds to C1s” refer to an antibody that is capable of binding C1s with sufficient affinity such that the antibody is useful as a diagnostic and/or therapeutic agent in targeting C1s. In one embodiment, the extent of binding of an anti-C1s antibody to an unrelated, non-C1s protein is less than about 10% of the binding of the antibody to C1s as measured, e.g., by a radioimmunoassay (RIA). In certain embodiments, an antibody that binds to C1s has a dissociation constant (Kd) of 1 micromolar (micro M) or less, 100 nM or less, 10 nM or less, 1 nM or less, 0.1 nM or less, 0.01 nM or less, or 0.001 nM or less (e.g. 10-8 M or less, e.g. from 10-8 M to 10-13 M, e.g., from 10-9 M to 10-13 M). In certain embodiments, an anti-C1s antibody binds to an epitope of C1s that is conserved among C1s from different species.

The terms “anti-C1r antibody” and “an antibody that binds to C1r” refer to an antibody that is capable of binding C1r with sufficient affinity such that the antibody is useful as a diagnostic and/or therapeutic agent in targeting C1r. In one embodiment, the extent of binding of an anti-C1r antibody to an unrelated, non-C1r protein is less than about 10% of the binding of the antibody to C1r as measured, e.g., by a radioimmunoassay (RIA). In certain embodiments, an antibody that binds to C1r has a dissociation constant (Kd) of 1 micromolar (micro M) or less, 100 nM or less, 10 nM or less, 1 nM or less, 0.1 nM or less, 0.01 nM or less, or 0.001 nM or less (e.g. 10-8 M or less, e.g. from 10-8 M to 10-13 M, e.g., from 10-9 M to 10-13 M). In certain embodiments, an anti-C1r antibody binds to an epitope of C1r that is conserved among C1r from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., 211At, 131I, 125I, 90Y, 186Re, 188Re, 153Sm, 212Bi, 32P, 212Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” includes any determinant capable of being bound by an antibody. An epitope is a region of an antigen that is bound by an antibody that targets that antigen, and includes specific amino acids that directly contact the antibody. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3 -H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda, Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).
   Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-C1s antibody” or “Isolated nucleic acid encoding an anti-C1r antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa and lambda, based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX® (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The phrase “specifically bind to”, as used herein, refers to an activity or a characteristic of an antibody to bind to an antigen of no interest at a level of binding that includes background (i.e., non-specific) binding but does not include significant (i.e., specific) binding. In other words, “specifically bind to” refers to an activity or a characteristic of an antibody to bind to an antigen of interest at a level of binding that includes significant (i.e., specific) binding in addition to or in place of background (i.e., non-specific) binding. The specificity can be measured by any methods mentioned in this specification or known in the art. The above-mentioned level of non-specific or background binding may be zero, or may not be zero but near zero, or may be very low enough to be technically neglected by those skilled in the art. For example, when a skilled person cannot detect or observe any significant (or relatively strong) signal for binding between the antibody and the antigen of no interest in a suitable binding assay, it can be said that the antibody “does not specifically bind to” the antigen of no interest. In contrast, when a skilled person can detect or observe any significant (or relatively strong) signal for binding between the antibody and the antigen of interest in a suitable binding assay, it can be said that the antibody “specifically binds to” the antigen of interest.

The term “C1s,” as used herein, refers to any native C1s from any vertebrate source, including mammals such as primates (e.g. humans) and rodents (e.g., mice and rats), unless otherwise indicated. The term encompasses “full-length” unprocessed C1s as well as any form of C1s that results from processing in the cell. The term also encompasses naturally occurring variants of C1s, e.g., splice variants or allelic variants. The amino acid sequence of an exemplary human C1s is shown in SEQ ID NO: 1. The amino acid sequences of an exemplary cynomolgus monkey, and rat C1s are shown in SEQ ID Nos: 3 and 2, respectively.

The term “C1r,” as used herein, refers to any native C1r from any vertebrate source, including mammals such as primates (e.g. humans) and rodents (e.g., mice and rats), unless otherwise indicated. The term encompasses “full-length” unprocessed C1r as well as any form of C1r that results from processing in the cell. The term also encompasses naturally occurring variants of C1r, e.g., splice variants or allelic variants. The amino acid sequence of an exemplary human C1r is shown in SEQ ID NO: 4. The amino acid sequences of an exemplary cynomolgus monkey, and rat C1r are shown in SEQ ID Nos: 5 and 6, respectively.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. COMPOSITIONS AND METHODS

In one aspect, the invention is based, in part, on an antibody that inhibits the interaction between C1q and C1r2s2 complex and uses thereof. In certain embodiments, antibodies that bind to C1s are provided. In certain embodiments, antibodies that bind to C1r are provided. Antibodies of the invention are useful, e.g., for the diagnosis or treatment of complement-mediated disease or disorder.

### A. Exemplary Anti-Complement Component Antibodies

In one aspect, the invention provides isolated antibodies that inhibit the interaction between C1q and C1r2s2 complex. In one aspect, the invention provides isolated antibodies having a displacement function such that the antibody binds to C1qrs complex and promotes dissociation of C1q from C1qrs complex. In one aspect, the invention provides isolated antibodies that bind to C1s. In one aspect, the invention provides isolated antibodies that bind to C1s, whose binding activity varies depending on the ion concentration. In certain embodiments, the binding activity of anti-C1s antibody varies depending on pH, i.e., hydrogen ion (proton) concentration. In certain embodiments, the binding activity of anti-C1s antibody varies depending on the calcium concentration. In certain embodiment, the binding activity of anti-C1s antibody varies depending on both pH and the calcium concentration. In another aspect, the invention provides isolated antibodies that bind to C1r. In one aspect, the invention provides isolated antibodies that bind to C1r, whose binding activity varies depending on the ion concentration. In certain embodiments, the binding activity of anti-C1r antibody varies depending on pH, i.e., hydrogen ion (proton) concentration. In certain embodiments, the binding activity of anti-C1r antibody varies depending on the calcium concentration. In certain embodiment, the binding activity of anti-C1r antibody varies depending on both pH and the calcium concentration.

Throughout the section of ‘Description of Embodiment’, the term “C1s” can be replaced with “C1r” except for the description related to sequences specific to anti-C1s antibodies and sequences and domains specific to C1s protein.

Such antibodies are expected to be especially superior as pharmaceuticals, because the dose and frequency of administration in patients can be reduced and as a result the total dosage can be reduced. Anti-C1s antibodies are expected to have superior safety profile compared to antibodies that bind to and remove C1qrs complexes from plasma, as they will only remove C1r2s2 (through the binding to C1s) from plasma not but C1q from plasma. As a result, side effects associated with C1q depletion can be avoided. In addition, antibodies with rapid displacement of C1q are expected to have faster neutralization of complement activity, which can translate to faster onset of treatment efficacy.

### (BIACORE®/Displacement Concept)

In one aspect, an isolated antibody that inhibits the interaction between C1q and C1r2s2 complex of the present invention is an antibody binding to C1qrs complex on a chip for surface plasmon resonance assay, e.g., a BIACORE (registered trademark) chip and promotes dissociation of C1q from C1qrs complex. In some aspects, a function of binding to C1qrs complex and promoting dissociation of C1q from C1qrs complex mentioned above is herein called “displacement function/activity” or “C1q displacement function/activity”. The function/activity can be suitable assessed qualitatively or quantitatively using a surface plasmon resonance assay, for example, a BIACORE® assay as described herein. In further aspects, the antibody of the present invention can be determined as an antibody having a displacement function when a value of response unit (RU) in presence of the antibody is lower than a value of response unit (RU) in the absence of the antibody as determined by surface plasmon resonance assay, for example a BIACORE® assay, when a sufficient time passed. In a sensorgram obtained from such an assay, one can identify a “time point of crossover” where the curve in the presence of C1q with the absence of the antibody crosses the curve in the presence of C1q and the antibody (see the Examples for detail). To be strict, multiple time points of crossover may be observed even in a single sensorgram due to noise or oscillation of the latter curve when crossing the former curve. In such a case, any of the multiple time points of crossover may be selected as the “time point of crossover”. “Passing a sufficient time” means that the time point of the measurement of the value of response unit (RU) is sufficiently after the “time point of crossover” for the purpose of the measurement. In some embodiments, the time point of the measurement of the value of response unit (RU) is at least 60 s, 100 s, 150 s, 200 s, 500 s, 700 s, 1000 s, 1500 s or 2000 s after the time point of the start of antibody injection. Alternatively, the time point of the measurement may be at least 100 s, 200 s, 300 s, 400 s, 500 s, 600 s, 700 s, 800 s, 900 s, 1000 s, 3000 s, 5000 s, 7000 s, or 10000 s after the time point of crossover.

In one aspect, an isolated antibody that inhibits the interaction between C1q and C1r2s2 complex of the present invention can be determined as an antibody having a displacement function when the time point of crossover (e.g., in a BIACORE® assay) is within 60 s, 100 s, 150 s, 200 s, 500 s, 700 s, 1000 s, 1500 s, or 2000 s after the time point of the start of antibody injection, as determined by, for example, a BIACORE® assay using the following conditions: The capture levels of C1r2s2 complex and C1q are at 200 resonance unit (RU) and 200 resonance unit (RU), respectively, and the antibody as an analyte is injected at 500 nM at 10 microliter (micro L)/min.

In one aspect, an isolated antibody that inhibits the interaction between C1q and C1r2s2 complex of the present invention can be determined as an antibody having a displacement function when almost all (or all) of C1q are dissociated from C1qrs complex within 100 s, 300 s, 500 s, 700 s, 1000 s, 1500 s, 2000 s, 3000 s, 5000 s, 7000 s, or 10000 s after the time point of the start of antibody injection, as determined by, for example a BIACORE® assay using the following conditions: The capture levels of C1r2s2 complex and C1q are at 200 resonance unit (RU) and 200 resonance unit (RU), respectively, and the antibody as an analyte is injected at 500 nM at 10 micro L/min. For example, in a sensorgram obtained from such an assay, it can be determined that “almost all (or all) of C1q are dissociated from C1qrs complex” when the value (RU) in the presence of C1q and the antibody comes close to or reaches the value (RU) in the presence of the antibody with the absence of C1q. Herein, “almost all (of C1q)” refers to a percentage of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more; and “all (of C1q)” refers to a percentage of 100%. The percentage of dissociated C1q may be quantitatively determined by any assay described herein. In some aspects, the present invention provides a method of screening for an antibody that displaces C1q from C1r2s2 complex, using the above-mentioned method of measuring the “displacement function/activity” of such an antibody. In one embodiment, the screening method comprises selecting an antibody that inhibits the interaction between C1q and C1r2s2 complex; i.e., selecting an antibody that binds to C1qrs complex and promotes dissociation of C1q from C1qrs complex. The antibody having the displacement function/activity can be suitable selected using a surface plasmon resonance assay, for example, a BIACORE® assay as described herein. In some embodiments, the screening method comprises determining (i) a value of response unit (RU) in presence of the antibody and (ii) a value of response unit (RU) in the absence of the antibody, by surface plasmon resonance assay, for example a BIACORE® assay, when a sufficient time passed. The screening method may comprise comparing the value of (i) above and the value of (ii) above. The screening method may comprise selecting the antibody when the value of (i) above is lower than the value of (ii) above. The screening method may comprise identifying a “time point of crossover” where the curve in the presence of C1q with the absence of the antibody crosses the curve in the presence of C1q and the antibody. As mentioned above, multiple time points of crossover may be observed even in a single sensorgram, and any of the multiple time points of crossover may be selected as the “time point of crossover”. In some embodiments, the screening method may comprise measuring the value of response unit (RU) at at least 60 s, 100 s, 150 s, 200 s, 500 s, 700 s, 1000 s, 1500 s or 2000 s after the time point of the start of antibody injection. Alternatively, the screening method may comprise measuring the value of response unit (RU) at at least 100 s, 200 s, 300 s, 400 s, 500 s, 600 s, 700 s, 800 s, 900 s, 1000 s, 3000 s, 5000 s, 7000 s, or 10000 s after the time point of crossover. In some embodiments, the screening method may comprise selecting an antibody that inhibits the interaction between C1q and C1r2s2 complex or an antibody having a displacement function, when the time point of crossover of the antibody is within 60 s, 100 s, 150 s, 200 s, 500 s, 700 s, 1000 s, 1500 s, or 2000 s after the time point of the start of antibody injection, as determined by, for example, a BIACORE® assay using the following conditions: The capture levels of C1r2s2 complex and C1q are at 200 resonance unit (RU) and 200 resonance unit (RU), respectively, and the antibody as an analyte is injected at 500 nM at 10 microliter (micro L)/min. In some embodiments, the screening method may comprise selecting an antibody that inhibits the interaction between C1q and C1r2s2 complex or an antibody having a displacement function, when almost all (or all) of C1q are dissociated from C1qrs complex within 100 s, 300 s, 500 s, 700 s, 1000 s, 1500 s, 2000 s, 3000 s, 5000 s, 7000 s, or 10000 s after the time point of the start of antibody injection, as determined by, for example a BIACORE® assay using the following conditions: The capture levels of C1r2s2 complex and C1q are at 200 resonance unit (RU) and 200 resonance unit (RU), respectively, and the antibody as an analyte is injected at 500 nM at 10 micro L/min. As mentioned above, “almost all (of C1q)” refers to a percentage of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, and “all (of C1q)” refers to 100%, and the percentage of dissociated C1q may be quantitatively determined by any assay described herein including a BIACORE® assay.

### ((BIACORE®)/Blocking Concept)

In one aspect, the invention provides isolated antibodies that inhibit the interaction between C1q and C1r2s2 complex, wherein the antibody has a blocking function such that the antibody binds to C1r2s2 and inhibits the binding of C1q to C1r2s2. In further aspect, the antibody of the present invention has the blocking ratio at least 60%, 65%, 70%, 75%, 80%, 85%, 90%, 95% or more. The blocking function/activity or blocking ratio can be determined by using a BIACORE® assay. The following conditions can be used for evaluating the level of C1q blocking: The capture levels of C1r2s2 are aimed at 50, 100, 200, 400 resonance unit (RU). Antibody variants are injected at 250, 500, 1000, 2000 nM to saturate antibody binding, followed by human C1q injection at 50, 100, 200 nM with or without antibody variants at 250, 500, 1000, 2000 nM. The blocking ratio is calculated by the following formula: [1−(human C1q binding response in the presence of antibody variant/human C1q binding response in the absence of antibody variant)]×100%.

### (pH Dependency)

In one aspect, an antibody of the present invention binds to the antigen such as C1s or bind to C1r2s2 complex in a pH-dependent manner. In a preferred embodiment, the present invention provides an isolated antibody that inhibits the interaction between C1q and C1r2s2 complex (through binding to C1s), where the antigen-binding activity (i.e., the binding activity to C1s) is lower at pH 5.8 than at pH 7.4. In a preferred embodiment, the antibody specifically binds to an epitope within a CUB1-EGF-CUB2 domain of C1s, wherein the antigen-binding activity of the antibody is lower at pH 5.8 than at pH 7.4.

In addition to binding to C1s in a pH-dependent manner, the effect of calcium on a pH-dependent antibody's affinity to C1s may be another important property. C1s forms dimers at high calcium concentrations but dissociates into monomers at low calcium concentrations. When C1s is in a dimeric state, a bivalent antibody is able to form immune complexes by crosslinking multiple C1s molecules. This allows the antibody to bind to C1s molecules within the complex by both affinity and avidity interactions, thus increasing the apparent affinity of the antibody. In contrast, when C1s is in a monomeric state, the antibody only binds by affinity interaction to C1s. This means that the pH-dependent C1s antibody can form immune complex with dimeric C1s in the plasma, but once within the acidic endosome, C1s will dissociate into monomers. This leads to the disassembly of the immune complex which then enhances the pH-dependent dissociation of the antibody from the antigen.

In one aspect, in an isolated anti-C1s antibody of the present invention, the ratio of the KD value for its C1s-binding activity at acidic pH to the KD value for the C1s-binding activity at neutral pH (KD(acidic pH)/KD(neutral pH)) is 2 or more when measured at a high calcium concentration at both neutral and acidic pH. In one aspect, in an isolated anti-C1s antibody of the present invention, the ratio of the KD value for its C1s-binding activity at acidic pH to the KD value for the C1s-binding activity at neutral pH (KD(acidic pH)/KD(neutral pH)) is 2 or more when measured at a high calcium concentration at neutral pH and at a low calcium concentration at acidic pH. In some embodiments, in an isolated anti-C1s antibody of the present invention, the ratio of the KD value for its C1s-binding activity at acidic pH to the KD value for the C1s-binding activity at neutral pH (KD(acidic pH)/KD(neutral pH)) is 2 or more when measured at a low calcium concentration at both neutral and acidic pH, wherein the anti-C1s antibody binds to the dimeric state of C1s.

Without being bound by a particular theory, in case that 1) an epitope structure of C1s bound by the antibody of the present invention can be conformationally changed by the non-existence of calcium thereby altering the affinity of the antibody or 2) the interaction (affinity or avidity) of the antibody of the present invention can vary depending on the condition of C1s (a monomeric state or a dimeric state), the measurement by using specific conditions (at a high calcium concentration at neutral pH and at a low calcium concentration at acidic pH) may be used to evaluate the ratio of the KD value (KD(acidic pH)/KD(neutral pH)).

In other words, the antibody of the present invention binds to C1s with a higher affinity at neutral pH than at acidic pH as described in (i) or (ii) below:
(i) when measured at a high calcium concentration at both neutral and acidic pH, the ratio of the KD value for C1s-binding activity at acidic pH to the KD value for C1s-binding activity at neutral pH (KD(acidic pH)/KD(neutral pH)) is 2 or more,
(ii) when measured at a high calcium concentration at neutral pH and at a low calcium concentration at acidic pH, the ratio of the KD value for C1s-binding activity at acidic pH to the KD value for C1s-binding activity at neutral pH (KD(acidic pH)/KD(neutral pH)) is 2 or more.

More generally, without being bound by a particular theory, in case that 1) an epitope structure of a certain antigen bound by an antibody of the present invention can be conformationally changed by the non-existence of calcium thereby altering the affinity of the antibody or 2) the interaction (affinity or avidity) of the antibody of the present invention can vary depending on the condition of the antigen (a monomeric state or a dimeric state), the measurement by using specific conditions (at a high calcium concentration at neutral pH and at a low calcium concentration at acidic pH) may be used to evaluate the ratio of the KD value (KD(acidic pH)/KD(neutral pH)).

Therefore, the antibody of the present invention binds to an antigen with a higher affinity at neutral pH than at acidic pH as follows: when measured at a high calcium concentration at neutral pH and at a low calcium concentration at acidic pH, the ratio of the KD value for antigen binding activity at acidic pH to the KD value for antigen binding activity at neutral pH (KD(acidic pH)/KD(neutral pH)) is 2 or more.

The above-mentioned KD ratio, i.e., KD(acidic pH)/KD(neutral pH) may be compared between the parent antibody (i.e., the original antibody before modification of this invention) and an antibody into which one or more amino acid mutations (e.g., additions, insertions, deletions, or substitution) have been introduced with respect to the original (parent) antibody. The original (parent) antibody may be any known or newly isolated antibody as long as it specifically binds to C1s. Thus, in one aspect, in an isolated anti-C1s antibody of the present invention, the ratio of the KD value for the C1s-binding activity at acidic pH to the KD value for the C1s-binding activity at neutral pH (KD(acidic pH)/KD(neutral pH)) is at least 1.2 times, 1.4 times, 1.6 times, 1.8 times, 2 times, 2.5 times, 3 times, 3.5 times, 4 times, 5 times, 8 times, 10 times higher than the ratio of the KD value for the C1s-binding activity at acidic pH to the KD value for the C1s-binding activity at neutral pH (KD(acidic pH)/KD(neutral pH)) of the original (parent) antibody. In other words, the present invention provides an isolated anti-C1s antibody wherein the isolated anti-C1s antibody has been introduced with one or more amino acid mutations (e.g., additions, insertions, deletions, or substitution) from a parent (original) antibody, and the ratio of (i) to (ii) below is at least 1.2, 1.4, 1.6, 1.8, 2, 2.5, 3, 3.5, 4, 5, 8, or 10: (i) the ratio of the KD value for the C1s-binding activity at acidic pH to the KD value for the C1s-binding activity at neutral pH (KD(acidic pH)/KD(neutral pH)) of the isolated anti-C1s antibody; (ii) the ratio of the KD value for the C1s-binding activity at acidic pH to the KD value for the C1s-binding activity at neutral pH (KD(acidic pH)/KD(neutral pH)) of the parent (original) antibody. These KD ratio may be measured at any (high or low) calcium concentration, e.g., measured at a high calcium concentration at both neutral and acidic pH, or measured at a high calcium concentration at neutral pH and at a low calcium concentration at acidic pH.

In one aspect, antibodies of the present invention have antigen-binding activity which is different between intracellular condition and extracellular condition. Intracellular and extracellular conditions refer to conditions that are different between inside and outside of the cell. Categories of conditions include, for example, ion concentration, more specifically, metal ion concentration, hydrogen ion concentration (pH) and calcium ion concentration. “Intracellular condition” preferably refers to an environment characteristic to the environment inside the endosome, while “extracellular condition” preferably refers to an environment characteristic to the environment in plasma. Antibodies with the property of having an antigen-binding activity that changes according to the ion concentration can be obtained by screening a large number of antibodies for domains having such property. For example, antibodies with the above-described property can be obtained by producing a large number of antibodies whose sequences are different from each another by a hybridoma method or an antibody library method, and measuring their antigen binding activities at different ion concentrations. The B cell cloning method is one of examples of methods of screening for such antibodies. Furthermore, as described below, at least one distinctive amino acid residue that can confer an antibody with the property of having an antigen-binding activity that changes according to the ion concentration is specified, to prepare as a library of a large number of antibodies that have different sequences while sharing the distinctive amino acid residues as a common structure. Such a library can be screened to efficiently isolate antibodies that have the property described above.

In one aspect, the invention provides an antibody that binds to C1s with a higher affinity at neutral pH than at acidic pH. In another aspect, the invention provides anti-C1s antibodies that exhibit pH-dependent binding to C1s. As used herein, the expression “pH-dependent binding” means “reduced binding at acidic pH as compared to at neutral pH”, and both expressions may be interchangeable. For example, anti-C1s antibodies “with pH-dependent binding characteristics” include antibodies that bind to C1s with higher affinity at neutral pH than at acidic pH.

In certain embodiment, the ratio of the KD value for C1s-binding activity at acidic pH to the KD value for C1s-binding activity at neutral pH (KD(acidic pH)/KD(neutral pH)) is 2 or more when measured at a high calcium concentration at both neutral and acidic pH. In particular embodiments, the antibodies of the present invention bind to C1s with at least 2, 3, 5, 10, 15, 20, 25, 30, 35, 40, 45, 50, 55, 60, 65, 70, 75, 80, 85, 90, 95, 100, 200, 400, 1000, 10000, or more times higher affinity at neutral pH than at acidic pH.

In certain embodiment, the ratio of the KD value for C1s-binding activity at acidic pH to the KD value for C1s-binding activity at neutral pH (KD(acidic pH)/KD(neutral pH)) is 2 or more when measured at a high calcium concentration at neutral pH and at a low calcium concentration at acidic pH. In particular embodiments, the antibodies of the present invention bind to C1s with at least 2, 3, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 200, 400, 1000, 10000, or more times higher affinity at neutral pH than at acidic pH.

In the above-mentioned cases, for example, acidic pH is 5.8 and neutral pH is 7.4, thus KD(acidic pH)/KD(neutral pH) is KD(pH 5.8)/KD(pH 7.4). In this connection, examples of acidic pH and neutral pH are herein described in detail later. In some embodiments, KD(acidic pH)/KD(neutral pH) such as KD(pH 5.8)/KD(pH 7.4) may be 2 to 10,000.

When an antigen is a soluble protein, the binding of an antibody to the antigen can result in an extended half-life of the antigen in plasma (i.e., reduced clearance of the antigen from plasma), since the antibody can have a longer half-life in plasma than the antigen itself and may serve as a carrier for the antigen. This is due to the recycling of the antigen-antibody complex by FcRn through the endosomal pathway in cell (Roopenian and Akilesh (2007) Nat Rev Immunol 7(9): 715-725). However, an antibody with pH-dependent binding characteristics, which binds to its antigen in neutral extracellular environment while releasing the antigen into acidic endosomal compartments following its entry into cells, is expected to have superior properties in terms of antigen neutralization and clearance relative to its counterpart that binds in a pH-independent manner (Igawa et al (2010) Nature Biotechnol 28(11); 1203-1207; Devanaboyina et al (2013) mAbs 5(6): 851-859; International Patent Application Publication No: WO 2009/125825).

In one aspect, the invention provides an antibody that binds to C1s with a higher affinity under a high calcium concentration condition than under a low calcium concentration condition.

In the present invention, preferred metal ions include, for example, calcium ion. Calcium ion is involved in modulation of many biological phenomena, including contraction of muscles such as skeletal, smooth, and cardiac muscles; activation of movement, phagocytosis, and the like of leukocytes; activation of shape change, secretion, and the like of platelets; activation of lymphocytes; activation of mast cells including secretion of histamine; cell responses mediated by catecholamine alpha receptor or acetylcholine receptor; exocytosis; release of transmitter substances from neuron terminals; and axoplasmic flow in neurons. Known intracellular calcium ion receptors include troponin C, calmodulin, parvalbumin, and myosin light chain, which have several calcium ion-binding sites and are believed to be derived from a common origin in terms of molecular evolution. There are also many known calcium-binding motifs. Such well-known motifs include, for example, cadherin domains, EF-hand of calmodulin, C2 domain of Protein kinase C, Gla domain of blood coagulation protein Factor IX, C-type lectins of acyaroglycoprotein receptor and mannose-binding receptor, A domains of LDL receptors, annexin, thrombospondin type 3 domain, and EGF-like domains.

In the present invention, when the metal ion is calcium ion, it is desirable that the antigen-binding activity is lower under a low calcium ion concentration condition than under a high calcium ion concentration condition. Meanwhile, the intracellular calcium ion concentration is lower than the extracellular calcium ion concentration. Conversely, the extracellular calcium ion concentration is higher than the intracellular calcium ion concentration. In the present invention, the low calcium ion concentration is preferably 0.1 micromolar (micro M) to 30 micro M, more preferably 0.5 micro M to 10 micro M, and particularly preferably 1 micro M to 5 micro M which is close to the calcium ion concentration in the early endosome in vivo. Meanwhile, in the present invention, the high calcium ion concentration is preferably 100 micro M to 10 micro M, more preferably 200 micro M to 5 mM, and particularly preferably 0.5 mM to 2.5 mM which is close to the calcium ion concentration in plasma (in blood). In the present invention, it is preferable that the low calcium ion concentration is the calcium ion concentration in endosomes, and the high calcium ion concentration is the calcium ion concentration in plasma. When the level of antigen-binding activity is compared between low and high calcium ion concentrations, it is preferable that the binding of antibodies of the present invention is stronger at a high calcium ion concentration than at a low calcium ion concentration. In other words, it is preferable that the antigen-binding activity of an antibody of the present invention is lower at a low calcium ion concentration ion than at a high calcium ion concentration. When the level of binding activity is expressed with the dissociation constant (KD), the value of KD (low calcium ion concentration)/KD (high calcium ion concentration) is greater than 1, preferably 2 or more, still more preferably 10 or more, and yet more preferably 40, 45, 50, 55, 60, 65, 70, 75, 80, 85, 90, 95, 100, 200, 400, 1000, 10000 or more. The upper limit of the value of KD (low calcium ion concentration)/KD (high calcium ion concentration) is not particularly limited, and may be any value such as 100, 400, 1000, or 10000, as long as it can be produced with the techniques of skilled artisans. It is possible to use the dissociation rate constant (kd) instead of KD. When it is difficult to calculate the KD value, the activity may be assessed based on the level of binding response in BIACORE® when analytes are passed at the same concentration. When antigens are passed over a chip immobilized with antigen-binding molecules of the present invention, the binding response at a low calcium concentration is preferably ½ or less of the binding response at a high calcium concentration, more preferably ⅓ or less, still more preferably ⅕ or less, and particularly preferably 1/10 or less. It is known that in general the in vivo extracellular calcium ion concentration (for example, in plasma) is high, and the intracellular calcium ion concentration (for example, in the endosome) is low. Thus, in the present invention, it is preferable that the extracellular condition is a high calcium ion concentration, and the intracellular condition is a low calcium ion concentration. When the property that the antigen-binding activity is lower under an intracellular calcium ion concentration condition than under an extracellular calcium ion concentration condition is conferred to an antigen-binding molecule (e.g., an antibody) of the present invention, antigens that have bound to the antigen-binding molecule of the present invention outside of the cell dissociate from the antigen-binding molecule of the present invention inside the cell, thereby enhancing antigen incorporation into the cell from the outside of the cell. Such antibodies, when administered to the living body, can reduce antigen concentration in plasma and reduce the physiological activity of antigens in vivo. Thus, antibodies of the present invention are useful. Methods of screening for antigen-binding domains or antibodies having a lower antigen-binding activity under a low calcium ion concentration condition than under a high calcium ion concentration condition include, for example, the method described in WO2012/073992 (for example, paragraphs 0200-0213). Methods for conferring antigen-binding domains of the present invention with the property of binding more weakly to antigens under a low calcium ion concentration condition than under a high calcium ion concentration condition are not particularly limited, and may be carried out by any methods. Specifically, the methods are described in Japanese Patent Application No. 2011-218006 and include, for example, methods for substituting at least one amino acid residue in an antigen-binding domain with an amino acid residue having metal chelating activity, and/or inserting into an antigen-binding domain at least one amino acid residue having metal chelating activity. Antigen-binding molecules of the present invention in which at least one amino acid residue of the antigen-binding domain has been substituted with an amino acid residue having metal chelating activity and/or at least one amino acid residue having metal chelating activity has been inserted into the antigen-binding domain are a preferred embodiment of antigen-binding molecules of the present invention.

Amino acid residues having metal chelating activity preferably include, for example, serine, threonine, asparagine, glutamine, aspartic acid, and glutamic acid. Furthermore, amino acid residues that change the antigen-binding activity of antigen binding domains according to the calcium ion concentration preferably include, for example, amino acid residues that form a calcium-binding motif. Calcium-binding motifs are well known to those skilled in the art, and have been described in detail (for example, Springer et al., (Cell (2000) 102, 275-277); Kawasaki and Kretsinger (Protein Prof (1995) 2, 305-490); Moncrief et al., (J. Mol. Evol. (1990) 30, 522-562); Chauvaux et al., (Biochem. J. (1990) 265, 261-265); Bairoch and Cox (FEBS Lett. (1990) 269, 454-456); Davis (New Biol. (1990) 2, 410-419); Schaefer et al., (Genomics (1995) 25, 638 to 643); Economou et al., (EMBO J. (1990) 9, 349-354); Wurzburg et al., (Structure. (2006) 14, 6, 1049-1058)). EF hand in troponin C, calmodulin, parvalbumin, and myosin light chain; C2 domain in protein kinase C; Gla domain in blood coagulation protein factor IX; C-type lectin of acyaroglycoprotein receptor and mannose-binding receptor, ASGPR, CD23, and DC-SIGN; A domain in LDL receptor; annexin domain; cadherin domain; thrombospondin type 3 domain; and EGF-like domain are preferably used as calciumbinding motifs.

Antigen-binding domains of the present invention can contain amino acid residues that change the antigen-binding activity according to the calcium ion concentration, such as the above-described amino acid residues with metal chelating activity and amino acid residues that form a calcium-binding motif. The location of such amino acid residues in the antigen-binding domain is not particularly limited, and they may be located at any position as long as the antigen binding activity changes according to the calcium ion concentration. Meanwhile, such amino acid residues may be contained alone or in combination of two or more, as long as the antigen binding activity changes according to the calcium ion concentration. The amino acid residues preferably include, for example, serine, threonine, asparagine, glutamine, aspartic acid, and glutamic acid. When an antigen-binding domain is an antibody variable region, the amino acid residues may be contained in the heavy chain variable region and/or the light chain variable region. In a preferred embodiment, the amino acid residues may be contained in the CDR3 of the heavy chain variable region, more preferably at positions 95, 96, 100a, and/or 101 according to Kabat numbering in the CDR3 of the heavy chain variable region.

In another preferred embodiment, the amino acid residues may be contained in the CDR1 of the light chain variable region, more preferably at positions 30, 31, and/or 32 according to Kabat numbering in the CDR1 of the light chain variable region. In still another preferred embodiment, the amino acid residues may be contained in the CDR2 of the light chain variable region, more preferably at position 50 according to Kabat numbering in the CDR2 of the light chain variable region. In yet another preferred embodiment, the amino acid residues may be contained in the CDR3 of the light chain variable region, more preferably at position 92 according to Kabat numbering in the CDR3 of the light chain variable region.

Furthermore, it is possible to combine the above-described embodiments. For example, the amino acid residues may be contained in two or three CDRs selected from the CDR1, CDR2, and CDR3 of the light chain variable region, more preferably at any one or more of positions 30, 31, 32, 50, and/or 92 according to Kabat numbering in the light chain variable region.

A large number of antigen-binding domains that have different sequences while sharing as a common structure the above-described amino acid residues that change the antigen-biding activity according to the calcium ion concentration, are prepared as a library. The library can be screened to efficiently obtain antigen-binding domains with binding activity to a desired antigen, in which their antigen-binding activity changes according to the calcium ion concentration.

The “affinity” of an antibody for C1s, for purposes of the present disclosure, is expressed in terms of the KD of the antibody. The KD of an antibody refers to the equilibrium dissociation constant of an antibody-antigen interaction. The greater the KD value is for an antibody binding to its antigen, the weaker its binding affinity is for that particular antigen. Accordingly, as used herein, the expression “higher affinity at neutral pH than at acidic pH” (or the equivalent expression “pH-dependent binding”) means that the KD of the antibody at acidic pH is greater than the KD of the antibody at neutral pH. For example, in the context of the present invention, an antibody is considered to bind to C1s with higher affinity at neutral pH than at acidic pH if the KD of the antibody binding to C1s at acidic pH is at least 2 times greater than the KD of the antibody binding to C1s at neutral pH. Thus, the present invention includes antibodies that bind to C1s at acidic pH with a KD that is at least 2, 3, 5, 10, 15, 20, 25, 30, 35, 40, 45, 50, 55, 60, 65, 70, 75, 80, 85, 90, 95, 100, 200, 400, 1000, 10000, or more times greater than the KD of the antibody binding to C1s at neutral pH. In another embodiment, the KD value of the antibody at neutral pH can be 10-7 M, 10-8 M, 10-9 M, 10-10 M, 10-11 M, 10-12 M, or less. In another embodiment, the KD value of the antibody at acidic pH can be 10-9 M, 10-8 M, 10-7 M, 10-6 M, or greater.

The binding properties of an antibody for a particular antigen may also be expressed in terms of the kd of the antibody. The kd of an antibody refers to the dissociation rate constant of the antibody with respect to a particular antigen and is expressed in terms of reciprocal seconds (i.e., sec-1). An increase in kd value signifies weaker binding of an antibody to its antigen. The present invention therefore includes antibodies that bind to C1s with a higher kd value at acidic pH than at neutral pH. The present invention includes antibodies that bind to C1s at acidic pH with a kd that is at least 2, 3, 5, 10, 15, 20, 25, 30, 35, 40, 45, 50, 55, 60, 65, 70, 75, 80, 85, 90, 95, 100, 200, 400, 1000, 10000, or more times greater than the kd of the antibody binding to C1s at neutral pH. In another embodiment, the kd value of the antibody at neutral pH can be 10-2 l/s, 10-3 l/s, 10-4 l/s, 10-5 l/s, 10-6 l/s, or less. In another embodiment, the kd value of the antibody at acidic pH can be 10-3 l/s, 10-2 l/s, 10-1 l/s, or greater.

In certain instances, a “reduced binding at acidic pH as compared to at neutral pH” is expressed in terms of the ratio of the KD value of the antibody at acidic pH to the KD value of the antibody at neutral pH (or vice versa). For example, an antibody may be regarded as exhibiting “reduced binding to C1s at acidic pH as compared to its binding at neutral pH”, for purposes of the present invention, if the antibody exhibits an acidic/neutral KD ratio of 2 or greater. In certain exemplary embodiments, the acidic/neutral KD ratio for an antibody of the present invention can be 2, 3, 5, 10, 15, 20, 25, 30, 35, 40, 45, 50, 55, 60, 65, 70, 75, 80, 85, 90, 95, 100, 200, 400, 1000, 10000, or greater. In another embodiment, the KD value of the antibody at neutral pH can be 10-7 M, 10-8 M, 10-9 M, 10-10 M, 10-11 M, 10-12 M, or less. In another embodiment, the KD value of the antibody at acidic pH can be 10-9 M, 10-8 M, 10-7 M, 10-6 M, or greater.

In certain instances, a “reduced binding at acidic pH as compared to at neutral pH” is expressed in terms of the ratio of the kd value of the antibody at acidic pH to the kd value of the antibody at neutral pH (or vice versa). For example, an antibody may be regarded as exhibiting “reduced binding to C1s at acidic pH as compared to its binding at neutral pH”, for purposes of the present invention, if the antibody exhibits an acidic/neutral kd ratio of 2 or greater. In certain exemplary embodiments, the acidic/neutral kd ratio for an antibody of the present invention can be 2, 3, 5, 10, 15, 20, 25, 30, 35, 40, 45, 50, 55, 60, 65, 70, 75, 80, 85, 90, 95, 100, 200, 400, 1000, 10000, or greater. In another embodiment, the kd value of the antibody at neutral pH can be 10-2 l/s, 10-3 l/s, 10-4 l/s, 10-5 l/s, 10-6 l/s, or less. In another embodiment, the kd value of the antibody at acidic pH can be 10-3 l/s, 10-2 l/s, 10-1 l/s, or greater.

As used herein, the expression “acidic pH” means a pH of 4.0 to 6.5. The expression “acidic pH” includes pH values of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, and 6.5. In particular aspects, the “acidic pH” is 5.8 or 6.0.

As used herein, the expression “neutral pH” means a pH of 6.7 to about 10.0. The expression “neutral pH” includes pH values of 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. In particular aspects, the “neutral pH” is 7.0 or 7.4.

As used herein, the expression “under high calcium concentration condition” or “at a high calcium concentration” means 100 micro M to 10 mM, more preferably 200 micro M to 5 mM, and particularly preferably 0.5 mM to 2.5 mM which is close to the calcium ion concentration in plasma (in blood). The expression “under high calcium concentration condition” or “at a high calcium concentration” includes calcium concentration values of 100 micro M, 200 micro M, 300 micro M, 400 micro M, 500 micro M, 600 micro M, 700 micro M, 800 micro M, 900 micro M, 0.5 mM, 0.7 mM, 0.9 mM, 1 mM, 1.2 mM, 1.4 mM, 1.6 mM, 1.8 mM, 2.0 mM, 2.2 mM, 2.4 mM, 2.5 mM, 3 mM, 4 mM, 5 mM, 6 mM, 7 mM, 8 mM, 9 mM, and 10 mM Ca2+. In particular aspects, “under high calcium concentration condition” or “at a high calcium concentration” refers to 1.2 mM Ca2+.

As used herein, the expression “under low calcium concentration condition” or “at a low calcium concentration” means 0.1 micro M to 30 micro M, more preferably 0.5 micro M to 10 micro M, and particularly preferably 1 micro M to 5 micro M which is close to the calcium ion concentration in the early endosome in vivo. The expression “under low calcium concentration condition” or “at a low calcium concentration” includes calcium concentration values of 0.1 micro M, 0.5 micro M, 1 micro M, 1.5 micro M, 2.0 micro M, 2.5 micro M, 2.6 micro M, 2.7 micro M, 2.8 micro M, 2.9 micro M, 3.0 micro M, 3.1 micro M, 3.2 micro M, 3.3 micro M, 3.4 micro M, 3.5 micro M, 4.0 micro M, 5.0 micro M, 6.0 micro M, 7.0 micro M, 8.0 micro M, 9.0 micro M, 10 micro M, 15 micro M, 20 micro M, 25 micro M, and 30 micro M Ca2+. In particular aspects, “under low calcium concentration condition” or “at a low calcium concentration” refers to 3.0 micro M Ca2+.

KD values and kd values, as expressed herein, may be determined using a surface plasmon resonance-based biosensor to characterize antibody-antigen interactions. (See, e.g., Example 2, herein). KD values and kd values can be determined at 25 degrees Celsius (C) or 37 degrees C. This determination can be performed in the presence of 150 mM NaCl. In some embodiments, this determination can be performed by using a surface plasmon resonance technique in which the antibody is immobilized, the antigen serves as analyte, and the following conditions are used: 10 mM MES buffer, 0.05% polyoxyethylenesorbitan monolaurate, and 150 mM NaCl at 37 degrees Celsius (C).

In one aspect, the invention provides a method of enhancing the clearance of C1s from plasma in an individual. In some embodiments, the method comprises administering to the individual an effective amount of an anti-C1s antibody of the present invention to enhance the clearance of C1s from plasma. The invention also provides a method of enhancing the clearance of the complex of C1r and C1s from plasma in an individual. In some embodiments, the method comprises administering to the individual an effective amount of an anti-C1s antibody of the present invention to enhance the clearance of the complex of C1r and C1s from plasma. In some embodiments, the method comprises administering to the individual an effective amount of an anti-C1s antibody of the present invention to enhance the clearance of C1r2s2 from plasma. In some embodiments, the method comprises administering to the individual an effective amount of an anti-C1s antibody of the present invention to enhance the clearance of C1r2s2 from plasma not but C1q from plasma.

In another aspect, the invention provides a method of removing C1s from plasma, the method comprising: (a) identifying an individual in need of having C1s removed from the individual's plasma; (b) providing an antibody that binds to C1s through the antigen-binding (C1s-binding) domain of the antibody and has a KD(pH 5.8)/KD(pH 7.4) value, defined as the ratio of KD for C1s at pH 5.8 and KD for C1s at pH 7.4, of 2 to 10,000, when KD is determined using a surface plasmon resonance technique, wherein the antibody binds to C1s in plasma in vivo and dissociates from the bound C1s under conditions present in an endosome in vivo, and wherein the antibody is a human IgG or a humanized IgG; and (c) administering the antibody to the individual. In further aspect, such a surface plasmon resonance technique can be used at 37 degrees C. and 150 mM NaCl. In further aspect, such a surface plasmon resonance technique can be used in which the antibody is immobilized, the antigen serves as analyte, and the following conditions are used: 10 mM MES buffer, 0.05% polyoxyethylenesorbitan monolaurate, and 150 mM NaCl at 37 degrees C.

In another aspect, the invention provides a method of removing C1s from plasma in a subject, the method comprising: (a) identifying a first antibody that binds to C1s through the antigen-binding domain of the first antibody; (b) identifying a second antibody that: (1) binds to C1s through the antigen-binding (C1s-binding) domain of the second antibody, (2) is identical in amino acid sequence to the first antibody except having at least one amino acid of a variable region of the first antibody substituted with histidine and/or at least one histidine inserted into a variable region of the first antibody, (3) has a KD(pH 5.8)/KD(pH 7.4) value that is higher than the first antibody's KD(pH 5.8)/KD(pH 7.4) value, and is between 2 and 10,000, wherein KD(pH 5.8)/KD(pH 7.4) is defined as the ratio of KD for C1s at pH 5.8 and KD for C1s at pH 7.4 when KD is determined using a surface plasmon resonance technique, (4) binds to C1s in plasma in vivo, (5) dissociates from the bound C1s under conditions present in an endosome in vivo, and (6) is a human IgG or a humanized IgG; (c) identifying a subject in need of having his or her plasma level of C1s reduced; and (d) administering the second antibody to the subject so that the plasma level of C1s in the subject is reduced. In further aspect, such a surface plasmon resonance technique can be used at 37 degrees C. and 150 mM NaCl. In further aspect, such a surface plasmon resonance technique can be used at 37 degrees C. and 150 mM NaCl. In further aspect, such a surface plasmon resonance technique can be used in which the antibody is immobilized, the antigen serves as analyte, and the following conditions are used: 10 mM MES buffer, 0.05% polyoxyethylenesorbitan monolaurate, and 150 mM NaCl at 37 degrees C.

In another aspect, the invention provides a method of removing C1s from plasma in a subject, the method comprising: (a) identifying a first antibody that: (1) binds to C1s through the antigen-binding domain of the first antibody, (2) is identical in amino acid sequence to a second antibody that binds to C1s through the antigen-binding (C1s-binding) domain of the second antibody, except that at least one variable region of the first antibody has at least one more histidine residue than does the corresponding variable region of the second antibody, (3) has a KD(pH 5.8)/KD(pH 7.4) value that is higher than the second antibody's KD(pH 5.8)/KD(pH 7.4) value, and is between 2 and 10,000, wherein KD(pH 5.8)/KD(pH 7.4) is defined as the ratio of KD for C1s at pH 5.8 and KD for C1s at pH 7.4 when KD is determined using a surface plasmon resonance technique, (4) binds to C1s in plasma in vivo, (5) dissociates from the bound C1s under conditions present in an endosome in vivo, and (6) is a human IgG or a humanized IgG; (b) identifying a subject in need of having his or her plasma level of C1s reduced; and (c) administering the first antibody at least once to the subject so that the plasma level of C1s in the subject is reduced. In further aspect, such a surface plasmon resonance technique can be used at 37 degrees C. and 150 mM NaCl. In further aspect, such a surface plasmon resonance technique can be used at 37 degrees C. and 150 mM NaCl. In further aspect, such a surface plasmon resonance technique can be used in which the antibody is immobilized, the antigen serves as analyte, and the following conditions are used: 10 mM MES buffer, 0.05% polyoxyethylenesorbitan monolaurate, and 150 mM NaCl at 37 degrees C. In some cases, the antibody inhibits a component of the classical complement pathway; in some cases, the classical complement pathway component is C1s.

In one aspect, the present disclosure provides a method to modulate complement activation. In some embodiments the method inhibits complement activation, for example to reduce production of C4b2a. In some embodiments, the present disclosure provides a method to modulate complement activation in an individual having a complement-mediated disease or disorder, the method comprising administering to the individual an anti-C1s antibody of the present disclosure or a pharmaceutical composition of the present disclosure, wherein the pharmaceutical composition comprises an anti-C1s antibody of the present disclosure. In some embodiments such a method inhibits complement activation. In some embodiments, the individual is a mammal. In some embodiments, the individual is a human. Administering can be by any route known to those skilled in the art, including those disclosed herein. In some embodiments, administering is intravenous. In some embodiments, administering is intrathecal.

In certain embodiments, an anti-C1s antibody of the present invention binds to C1s from more than one species. In particular embodiments, the anti-C1s antibody binds to C1s from a human and non-human animal. In particular embodiments, the anti-C1s antibody binds to C1s from human, rat, and monkey (e.g. cynomolgus, rhesus macaque, marmoset, chimpanzee, and baboon).

In one aspect, the invention provides an anti-C1s antibody comprising at least one, two, three, four, five, or six HVRs selected from (a) HVR-H1 comprising the amino acid sequence of SEQ ID NO: 32, 38, 44, 50, or 56; (b) HVR-H2 comprising the amino acid sequence of SEQ ID NO: 33, 39, 45, 51, or 57; (c) HVR-H3 comprising the amino acid sequence of SEQ ID NO: 34, 40, 46, 52, or 58; (d) HVR-L1 comprising the amino acid sequence of SEQ ID NO: 35, 41, 47, 53, or 59; (e) HVR-L2 comprising the amino acid sequence of SEQ ID NO: 36, 42, 48, 54, or 60; and (f) HVR-L3 comprising the amino acid sequence of SEQ ID NO: 37, 43, 49, 55, or 61.

In one aspect, the invention provides an anti-C1s antibody comprising at least one, at least two, or all three VH HVR sequences selected from (a) HVR-H1 comprising the amino acid sequence of SEQ ID NO: 32, 38, 44, 50, or 56; (b) HVR-H2 comprising the amino acid sequence of SEQ ID NO: 33, 39, 45, 51, or 57; and (c) HVR-H3 comprising the amino acid sequence of SEQ ID NO: 34, 40, 46, 52, or 58. In one embodiment, the antibody comprises HVR-H3 comprising the amino acid sequence of SEQ ID NO: 34, 40, 46, 52, or 58. In another embodiment, the antibody comprises HVR-H3 comprising the amino acid sequence of SEQ ID NO: 34, 40, 46, 52, or 58 and HVR-L3 comprising the amino acid sequence of SEQ ID NO: 37, 43, 49, 55, or 61. In a further embodiment, the antibody comprises HVR-H3 comprising the amino acid sequence of SEQ ID NO: 34, 40, 46, 52, or 58, HVR-L3 comprising the amino acid sequence of SEQ ID NO: 37, 43, 49, 55, or 61, and HVR-H2 comprising the amino acid sequence of SEQ ID NO: 33, 39, 45, 51, or 57. In a further embodiment, the antibody comprises (a) HVR-H1 comprising the amino acid sequence of SEQ ID NO: 32, 38, 44, 50, or 56; (b) HVR-H2 comprising the amino acid sequence of SEQ ID NO: 33, 39, 45, 51, or 57; and (c) HVR-H3 comprising the amino acid sequence of SEQ ID NO: 34, 40, 46, 52, or 58.

In another aspect, the invention provides an anti-C1s antibody comprising at least one, at least two, or all three VL HVR sequences selected from (a) HVR-L1 comprising the amino acid sequence of SEQ ID NO: 35, 41, 47, 53, or 59; (b) HVR-L2 comprising the amino acid sequence of SEQ ID NO: 36, 42, 48, 54, or 60; and (c) HVR-L3 comprising the amino acid sequence of SEQ ID NO: 37, 43, 49, 55, or 61. In one embodiment, the antibody comprises (a) HVR-L1 comprising the amino acid sequence of SEQ ID NO: 35, 41, 47, 53, or 59; (b) HVR-L2 comprising the amino acid sequence of SEQ ID NO: 36, 42, 48, 54, or 60; and (c) HVR-L3 comprising the amino acid sequence of SEQ ID NO: 37, 43, 49, 55, or 61.

In another aspect, an anti-C1s antibody of the invention comprises (a) a VH domain comprising at least one, at least two, or all three VH HVR sequences selected from (i) HVR-H1 comprising the amino acid sequence of SEQ ID NO: 32, 38, 44, 50, or 56, (ii) HVR-H2 comprising the amino acid sequence of SEQ ID NO: 33, 39, 45, 51, or 57, and (iii) HVR-H3 comprising an amino acid sequence of SEQ ID NO: 34, 40, 46, 52, or 58; and (b) a VL domain comprising at least one, at least two, or all three VL HVR sequences selected from (i) HVR-L1 comprising the amino acid sequence of SEQ ID NO: 35, 41, 47, 53, or 59, (ii) HVR-L2 comprising the amino acid sequence of SEQ ID NO: 36, 42, 48, 54, or 60, and (c) HVR-L3 comprising the amino acid sequence of SEQ ID NO: 37, 43, 49, 55, or 61.

In some embodiments, anti-C1s antibody variants which are prepared by introducing amino acid modifications into an antibody comprising a VH sequence of SEQ ID No: 19, 20, 21, 23, or 24 and a VL sequence of SEQ ID NO: 26, 27, 28, 30, or 31 are provided.

In some embodiments, anti-C1s antibody of the present invention comprises a histidine at one or more of the following Kabat numbering system positions: Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In some embodiments, anti-C1s antibody of the present invention comprises at least one histidine substituted for one or more amino acid residues at positions selected from the following Kabat numbering system positions: Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In any of the above embodiments, an anti-C1s antibody is humanized. In one embodiment, an anti-C1s antibody comprises HVRs as in any of the above embodiments, and further comprises an acceptor human framework, e.g. a human immunoglobulin framework or a human consensus framework. In another embodiment, an anti-C1s antibody comprises HVRs as in any of the above embodiments, and further comprises a VH or VL comprising an FR sequence. In a further embodiment, the anti-C1s antibody of the invention comprises the following heavy chain or light chain variable domain FR sequences

In another aspect, an anti-C1s antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19, 20, 21, 23, or 24. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C1s antibody comprising that sequence retains the ability to bind to C1s. In certain embodiments, a total of 1 to 10 amino acids have been substituted, inserted and/or deleted in SEQ ID NO: 19, 20, 21, 23, or 24. In certain embodiments, substitutions, insertions, or deletions occur in regions outside the HVRs (i.e., in the FRs). Optionally, the anti-C1s antibody comprises the VH sequence in SEQ ID NO: 19, 20, 21, 23, or 24, including post-translational modifications of that sequence. In a particular embodiment, the VH comprises one, two or three HVRs selected from: (a) HVR-H1 comprising the amino acid sequence of SEQ ID NO: 32, 38, 44, 50, or 56, (b) HVR-H2 comprising the amino acid sequence of SEQ ID NO: 33, 39, 45, 51, or 57, and (c) HVR-H3 comprising the amino acid sequence of SEQ ID NO: 34, 40, 46, 52, or 58. Post-translational modifications include but are not limited to a modification of glutamine or glutamate in N-terminal of heavy chain or light chain to pyroglutamic acid by pyroglutamylation.

In another aspect, an anti-C1s antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 26, 27, 28, 30, or 31. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C1s antibody comprising that sequence retains the ability to bind to C1s. In certain embodiments, a total of 1 to 10 amino acids have been substituted, inserted and/or deleted in SEQ ID NO: 26, 27, 28, 30, or 31. In certain embodiments, the substitutions, insertions, or deletions occur in regions outside the HVRs (i.e., in the FRs). Optionally, the anti-C1s antibody comprises the VL sequence in SEQ ID NO: 26, 27, 28, 30, or 31, including post-translational modifications of that sequence. In a particular embodiment, the VL comprises one, two or three HVRs selected from (a) HVR-L1 comprising the amino acid sequence of SEQ ID NO: 35, 41, 47, 53, or 59; (b) HVR-L2 comprising the amino acid sequence of SEQ ID NO: 36, 42, 48, 54, or 60; and (c) HVR-L3 comprising the amino acid sequence of SEQ ID NO: 37, 43, 49, 55, or 61. Post-translational modifications include but are not limited to a modification of glutamine or glutamate in N-terminal of heavy chain or light chain to pyroglutamic acid by pyroglutamylation.

In another aspect, an anti-C1s antibody is provided, wherein the antibody comprises a VH as in any of the embodiments provided above, and a VL as in any of the embodiments provided above. In one embodiment, the antibody comprises the VH and VL sequences in SEQ ID NO: 19 and SEQ ID NO: 26, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 20 and SEQ ID NO: 27, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 21 and SEQ ID NO: 28, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 23 and SEQ ID NO: 30, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 24 and SEQ ID NO: 31, respectively, including post-translational modifications of those sequences. Post-translational modifications include but are not limited to a modification of glutamine or glutamate in N-terminal of heavy chain or light chain to pyroglutamic acid by pyroglutamylation.

In a further aspect, the invention provides an antibody that binds to the same epitope as an anti-C1s antibody provided herein. In a preferred aspect, the antibody specifically binds to the same epitope as an anti-C1s antibody provided herein. For example, in certain embodiments, an antibody is provided that (specifically) binds to the same epitope as an antibody selected from the group consisting of:
1) an antibody comprising the HVR-H1 sequence of SEQ ID NO: 32, the HVR-H2 sequence of SEQ ID NO: 33, the HVR-H3 sequence of SEQ ID NO: 34, the HVR-L1 sequence of SEQ ID NO: 35, the HVR-L2 sequence of SEQ ID NO: 36, and the HVR-L3 sequence of SEQ ID NO: 37,
2) an antibody comprising the HVR-H1 sequence of SEQ ID NO: 38, the HVR-H2 sequence of SEQ ID NO: 39, the HVR-H3 sequence of SEQ ID NO: 40, the HVR-L1 sequence of SEQ ID NO: 41, the HVR-L2 sequence of SEQ ID NO: 42, and the HVR-L3 sequence of SEQ ID NO: 43,
3) an antibody comprising the HVR-H1 sequence of SEQ ID NO: 44, the HVR-H2 sequence of SEQ ID NO: 45, the HVR-H3 sequence of SEQ ID NO: 46, the HVR-L1 sequence of SEQ ID NO: 47, the HVR-L2 sequence of SEQ ID NO: 48, and the HVR-L3 sequence of SEQ ID NO: 49,
4) an antibody comprising the HVR-H1 sequence of SEQ ID NO: 50, the HVR-H2 sequence of SEQ ID NO: 51, the HVR-H3 sequence of SEQ ID NO: 52, the HVR-L1 sequence of SEQ ID NO: 53, the HVR-L2 sequence of SEQ ID NO: 54, and the HVR-L3 sequence of SEQ ID NO: 55, and
5) an antibody comprising the HVR-H1 sequence of SEQ ID NO: 56, the HVR-H2 sequence of SEQ ID NO: 57, the HVR-H3 sequence of SEQ ID NO: 58, the HVR-L1 sequence of SEQ ID NO: 59, the HVR-L2 sequence of SEQ ID NO: 60, and the HVR-L3 sequence of SEQ ID NO: 61.

In some embodiments, an isolated anti-C1s antibody of the present invention competes for binding to C1s with an antibody selected from the group consisting of 1) to 5) below. In some embodiments, an isolated anti-C1s antibody of the present invention competes at neutral pH for binding to C1s with an antibody selected from the group consisting of 1) to 5) below:
1) an antibody comprising the HVR-H1 sequence of SEQ ID NO: 32, the HVR-H2 sequence of SEQ ID NO: 33, the HVR-H3 sequence of SEQ ID NO: 34, the HVR-L1 sequence of SEQ ID NO: 35, the HVR-L2 sequence of SEQ ID NO: 36, and the HVR-L3 sequence of SEQ ID NO: 37,
2) an antibody comprising the HVR-H1 sequence of SEQ ID NO: 38, the HVR-H2 sequence of SEQ ID NO: 39, the HVR-H3 sequence of SEQ ID NO: 40, the HVR-L1 sequence of SEQ ID NO: 41, the HVR-L2 sequence of SEQ ID NO: 42, and the HVR-L3 sequence of SEQ ID NO: 43,
3) an antibody comprising the HVR-H1 sequence of SEQ ID NO: 44, the HVR-H2 sequence of SEQ ID NO: 45, the HVR-H3 sequence of SEQ ID NO: 46, the HVR-L1 sequence of SEQ ID NO: 47, the HVR-L2 sequence of SEQ ID NO: 48, and the HVR-L3 sequence of SEQ ID NO: 49,
4) an antibody comprising the HVR-H1 sequence of SEQ ID NO: 50, the HVR-H2 sequence of SEQ ID NO: 51, the HVR-H3 sequence of SEQ ID NO: 52, the HVR-L1 sequence of SEQ ID NO: 53, the HVR-L2 sequence of SEQ ID NO: 54, and the HVR-L3 sequence of SEQ ID NO: 55, and
5) an antibody comprising the HVR-H1 sequence of SEQ ID NO: 56, the HVR-H2 sequence of SEQ ID NO: 57, the HVR-H3 sequence of SEQ ID NO: 58, the HVR-L1 sequence of SEQ ID NO: 59, the HVR-L2 sequence of SEQ ID NO: 60, and the HVR-L3 sequence of SEQ ID NO: 61.

In one aspect, the invention provides an anti-C1r antibody comprising at least one, two, three, four, five, or six HVRs selected from (a) HVR-H1 comprising the amino acid sequence of SEQ ID NO: 119, 120, 121, 122, 123, 124, 125, or 126; (b) HVR-H2 comprising the amino acid sequence of SEQ ID NO: 127, 128, 129, 130, 131, 132, 133, or 134; (c) HVR-H3 comprising the amino acid sequence of SEQ ID NO: 135, 136, 137, 138, 139, 140, 141, or 142; (d) HVR-L1 comprising the amino acid sequence of SEQ ID NO: 143, 144, 145, 146, 147, 148, 149, or 150; (e) HVR-L2 comprising the amino acid sequence of SEQ ID NO: 151, 152, 153, 154, 155, 156, 157, or 158; and (0 HVR-L3 comprising the amino acid sequence of SEQ ID NO: 159, 160, 161, 162, 163, 164, 165, or 166.

In one aspect, the invention provides an anti-C1r antibody comprising at least one, at least two, or all three VH HVR sequences selected from (a) HVR-H1 comprising the amino acid sequence of SEQ ID NO: 119, 120, 121, 122, 123, 124, 125, or 126; (b) HVR-H2 comprising the amino acid sequence of SEQ ID NO: 127, 128, 129, 130, 131, 132, 133, or 134; and (c) HVR-H3 comprising the amino acid sequence of SEQ ID NO: 135, 136, 137, 138, 139, 140, 141, or 142. In one embodiment, the antibody comprises HVR-H3 comprising the amino acid sequence of SEQ ID NO: 135, 136, 137, 138, 139, 140, 141, or 142. In another embodiment, the antibody comprises HVR-H3 comprising the amino acid sequence of SEQ ID NO: 135, 136, 137, 138, 139, 140, 141, or 142 and HVR-L3 comprising the amino acid sequence of SEQ ID NO: 159, 160, 161, 162, 163, 164, 165, or 166. In a further embodiment, the antibody comprises HVR-H3 comprising the amino acid sequence of SEQ ID NO: 135, 136, 137, 138, 139, 140, 141, or 142, HVR-L3 comprising the amino acid sequence of SEQ ID NO: 159, 160, 161, 162, 163, 164, 165, or 166, and HVR-H2 comprising the amino acid sequence of SEQ ID NO: 127, 128, 129, 130, 131, 132, 133, or 134. In a further embodiment, the antibody comprises (a) HVR-H1 comprising the amino acid sequence of SEQ ID NO: 119, 120, 121, 122, 123, 124, 125, or 126; (b) HVR-H2 comprising the amino acid sequence of SEQ ID NO: 127, 128, 129, 130, 131, 132, 133, or 134; and (c) HVR-H3 comprising the amino acid sequence of SEQ ID NO: 135, 136, 137, 138, 139, 140, 141, or 142.

In another aspect, the invention provides an anti-C1r antibody comprising at least one, at least two, or all three VL HVR sequences selected from (a) HVR-L1 comprising the amino acid sequence of SEQ ID NO: 143, 144, 145, 146, 147, 148, 149, or 150; (b) HVR-L2 comprising the amino acid sequence of SEQ ID NO: 151, 152, 153, 154, 155, 156, 157, or 158; and (c) HVR-L3 comprising the amino acid sequence of SEQ ID NO: 159, 160, 161, 162, 163, 164, 165, or 166. In one embodiment, the antibody comprises (a) HVR-L1 comprising the amino acid sequence of SEQ ID NO: 143, 144, 145, 146, 147, 148, 149, or 150; (b) HVR-L2 comprising the amino acid sequence of SEQ ID NO: 151, 152, 153, 154, 155, 156, 157, or 158; and (c) HVR-L3 comprising the amino acid sequence of SEQ ID NO: 159, 160, 161, 162, 163, 164, 165, or 166.

In another aspect, an anti-C1r antibody of the invention comprises (a) a VH domain comprising at least one, at least two, or all three VH HVR sequences selected from (i) HVR-H1 comprising the amino acid sequence of SEQ ID NO: 119, 120, 121, 122, 123, 124, 125, or 126, (ii) HVR-H2 comprising the amino acid sequence of SEQ ID NO: 127, 128, 129, 130, 131, 132, 133, or 134, and (iii) HVR-H3 comprising an amino acid sequence of SEQ ID NO: 135, 136, 137, 138, 139, 140, 141, or 142; and (b) a VL domain comprising at least one, at least two, or all three VL HVR sequences selected from (i) HVR-L1 comprising the amino acid sequence of SEQ ID NO: 143, 144, 145, 146, 147, 148, 149, or 150, (ii) HVR-L2 comprising the amino acid sequence of SEQ ID NO: 151, 152, 153, 154, 155, 156, 157, or 158, and (c) HVR-L3 comprising the amino acid sequence of SEQ ID NO: 159, 160, 161, 162, 163, 164, 165, or 166.

In some embodiments, anti-C1r antibody variants which are prepared by introducing amino acid modifications into an antibody comprising a VH sequence of SEQ ID No: 103, 104, 105, 106, 107, 108, 109, or 110 and a VL sequence of SEQ ID NO: 111, 112, 113, 114, 115, 116, 117, or 118 are provided.

In some embodiments, anti-C1r antibody of the present invention comprises a histidine at one or more of the following Kabat numbering system positions:
Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In some embodiments, anti-C1r antibody of the present invention comprises at least one histidine substituted for one or more amino acid residues at positions selected from the following Kabat numbering system positions:
Heavy chain: H26, H27, H28, H29, H30, H31, H32, H33, H34, H35, H50, H51, H52, H52a, H53, H54, H55, H57, H58, H59, H60, H61, H62, H63, H64, H65, H93, H94, H95, H96, H97, H98, H99, H100, H100a, H101, and H102; and Light chain: L24, L25, L26, L27, L27a, L28, L29, L30, L31, L32, L33, L50, L51, L52, L53, L54, L55, L56 L91, L92, L93, L94, L95, L95a, L96, and L97.

In any of the above embodiments, an anti-C1r antibody is humanized. In one embodiment, an anti-C1r antibody comprises HVRs as in any of the above embodiments, and further comprises an acceptor human framework, e.g. a human immunoglobulin framework or a human consensus framework. In another embodiment, an anti-C1r antibody comprises HVRs as in any of the above embodiments, and further comprises a VH or VL comprising an FR sequence. In a further embodiment, the anti-C1r antibody of the invention comprises the following heavy chain or light chain variable domain FR sequences

In another aspect, an anti-C1r antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 103, 104, 105, 106, 107, 108, 109, or 110. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C1r antibody comprising that sequence retains the ability to bind to C1r. In certain embodiments, a total of 1 to 10 amino acids have been substituted, inserted and/or deleted in SEQ ID NO: 103, 104, 105, 106, 107, 108, 109, or 110. In certain embodiments, substitutions, insertions, or deletions occur in regions outside the HVRs (i.e., in the FRs). Optionally, the anti-C1r antibody comprises the VH sequence in SEQ ID NO: 103, 104, 105, 106, 107, 108, 109, or 110, including post-translational modifications of that sequence. In a particular embodiment, the VH comprises one, two or three HVRs selected from: (a) HVR-H1 comprising the amino acid sequence of SEQ ID NO: 119, 120, 121, 122, 123, 124, 125, or 126, (b) HVR-H2 comprising the amino acid sequence of SEQ ID NO: 127, 128, 129, 130, 131, 132, 133, or 134, and (c) HVR-H3 comprising the amino acid sequence of SEQ ID NO: 135, 136, 137, 138, 139, 140, 141, or 142. Post-translational modifications include but are not limited to a modification of glutamine or glutamate in N-terminal of heavy chain or light chain to pyroglutamic acid by pyroglutamylation.

In another aspect, an anti-C1r antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 111, 112, 113, 114, 115, 116, 117, or 118. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C1r antibody comprising that sequence retains the ability to bind to C1r. In certain embodiments, a total of 1 to 10 amino acids have been substituted, inserted and/or deleted in SEQ ID NO: 111, 112, 113, 114, 115, 116, 117, or 118. In certain embodiments, the substitutions, insertions, or deletions occur in regions outside the HVRs (i.e., in the FRs). Optionally, the anti-C1r antibody comprises the VL sequence in SEQ ID NO: 111, 112, 113, 114, 115, 116, 117, or 118, including post-translational modifications of that sequence. In a particular embodiment, the VL comprises one, two or three HVRs selected from (a) HVR-L1 comprising the amino acid sequence of SEQ ID NO: 143, 144, 145, 146, 147, 148, 149, or 150; (b) HVR-L2 comprising the amino acid sequence of SEQ ID NO: 151, 152, 153, 154, 155, 156, 157, or 158; and (c) HVR-L3 comprising the amino acid sequence of SEQ ID NO: 159, 160, 161, 162, 163, 164, 165, or 166. Post-translational modifications include but are not limited to a modification of glutamine or glutamate in N-terminal of heavy chain or light chain to pyroglutamic acid by pyroglutamylation.

In another aspect, an anti-C1r antibody is provided, wherein the antibody comprises a VH as in any of the embodiments provided above, and a VL as in any of the embodiments provided above. In one embodiment, the antibody comprises the VH and VL sequences in SEQ ID NO: 103 and SEQ ID NO: 111, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 104 and SEQ ID NO: 112, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 105 and SEQ ID NO: 113, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 106 and SEQ ID NO: 114, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 107 and SEQ ID NO: 115, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 108 and SEQ ID NO: 116, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 109 and SEQ ID NO: 117, respectively, including post-translational modifications of those sequences. Post-translational modifications include but are not limited to a modification of glutamine or glutamate in N-terminal of heavy chain or light chain to pyroglutamic acid by pyroglutamylation. In one embodiment, the antibody comprises the VH and VL sequences in SEQ ID NO: 110 and SEQ ID NO: 118, respectively, including post-translational modifications of those sequences. Post-translational modifications include but are not limited to a modification of glutamine or glutamate in N-terminal of heavy chain or light chain to pyroglutamic acid by pyroglutamylation.

In a further aspect, the invention provides an antibody that binds to the same epitope as an anti-C1r antibody provided herein. In a preferred aspect, the antibody specifically binds to the same epitope as an anti-C1r antibody provided herein. For example, in certain embodiments, an antibody is provided that (specifically) binds to the same epitope as an antibody selected from the group consisting of:
6) an antibody comprising the HVR-H1 sequence of SEQ ID NO: 119, the HVR-H2 sequence of SEQ ID NO: 127, the HVR-H3 sequence of SEQ ID NO: 135, the HVR-L1 sequence of SEQ ID NO: 143, the HVR-L2 sequence of SEQ ID NO: 151, and the HVR-L3 sequence of SEQ ID NO: 159,
7) an antibody comprising the HVR-H1 sequence of SEQ ID NO: 120, the HVR-H2 sequence of SEQ ID NO: 128, the HVR-H3 sequence of SEQ ID NO: 136, the HVR-L1 sequence of SEQ ID NO: 144, the HVR-L2 sequence of SEQ ID NO: 152, and the HVR-L3 sequence of SEQ ID NO: 160,
8) an antibody comprising the HVR-H1 sequence of SEQ ID NO: 121, the HVR-H2 sequence of SEQ ID NO: 129, the HVR-H3 sequence of SEQ ID NO: 137, the HVR-L1 sequence of SEQ ID NO: 145, the HVR-L2 sequence of SEQ ID NO: 153, and the HVR-L3 sequence of SEQ ID NO: 161,
9) an antibody comprising the HVR-H1 sequence of SEQ ID NO: 122, the HVR-H2 sequence of SEQ ID NO: 130, the HVR-H3 sequence of SEQ ID NO: 138, the HVR-L1 sequence of SEQ ID NO: 146, the HVR-L2 sequence of SEQ ID NO: 154, and the HVR-L3 sequence of SEQ ID NO: 162,
10) an antibody comprising the HVR-H1 sequence of SEQ ID NO: 123, the HVR-H2 sequence of SEQ ID NO: 131, the HVR-H3 sequence of SEQ ID NO: 139, the HVR-L1 sequence of SEQ ID NO: 147, the HVR-L2 sequence of SEQ ID NO: 155, and the HVR-L3 sequence of SEQ ID NO: 163,
11) an antibody comprising the HVR-H1 sequence of SEQ ID NO: 124, the HVR-H2 sequence of SEQ ID NO: 132, the HVR-H3 sequence of SEQ ID NO: 140, the HVR-L1 sequence of SEQ ID NO: 148, the HVR-L2 sequence of SEQ ID NO: 156, and the HVR-L3 sequence of SEQ ID NO: 164,
12) an antibody comprising the HVR-H1 sequence of SEQ ID NO: 125, the HVR-H2 sequence of SEQ ID NO: 133, the HVR-H3 sequence of SEQ ID NO: 141, the HVR-L1 sequence of SEQ ID NO: 149, the HVR-L2 sequence of SEQ ID NO: 157, and the HVR-L3 sequence of SEQ ID NO: 165, and
13) an antibody comprising the HVR-H1 sequence of SEQ ID NO: 126, the HVR-H2 sequence of SEQ ID NO: 134, the HVR-H3 sequence of SEQ ID NO: 142, the HVR-L1 sequence of SEQ ID NO: 150, the HVR-L2 sequence of SEQ ID NO: 158, and the HVR-L3 sequence of SEQ ID NO: 166.

In some embodiments, an isolated anti-C1r antibody of the present invention competes for binding to C1r with an antibody selected from the group consisting of 6) to 13) below. In some embodiments, an isolated anti-C1r antibody of the present invention competes at neutral pH for binding to C1r with an antibody selected from the group consisting of 6) to 13) below:
6) an antibody comprising the HVR-H1 sequence of SEQ ID NO: 119, the HVR-H2 sequence of SEQ ID NO: 127, the HVR-H3 sequence of SEQ ID NO: 135, the HVR-L1 sequence of SEQ ID NO: 143, the HVR-L2 sequence of SEQ ID NO: 151, and the HVR-L3 sequence of SEQ ID NO: 159,
7) an antibody comprising the HVR-H1 sequence of SEQ ID NO: 120, the HVR-H2 sequence of SEQ ID NO: 128, the HVR-H3 sequence of SEQ ID NO: 136, the HVR-L1 sequence of SEQ ID NO: 144, the HVR-L2 sequence of SEQ ID NO: 152, and the HVR-L3 sequence of SEQ ID NO: 160,
8) an antibody comprising the HVR-H1 sequence of SEQ ID NO: 121, the HVR-H2 sequence of SEQ ID NO: 129, the HVR-H3 sequence of SEQ ID NO: 137, the HVR-L1 sequence of SEQ ID NO: 145, the HVR-L2 sequence of SEQ ID NO: 153, and the HVR-L3 sequence of SEQ ID NO: 161,
9) an antibody comprising the HVR-H1 sequence of SEQ ID NO: 122, the HVR-H2 sequence of SEQ ID NO: 130, the HVR-H3 sequence of SEQ ID NO: 138, the HVR-L1 sequence of SEQ ID NO: 146, the HVR-L2 sequence of SEQ ID NO: 154, and the HVR-L3 sequence of SEQ ID NO: 162,
10) an antibody comprising the HVR-H1 sequence of SEQ ID NO: 123, the HVR-H2 sequence of SEQ ID NO: 131, the HVR-H3 sequence of SEQ ID NO: 139, the HVR-L1 sequence of SEQ ID NO: 147, the HVR-L2 sequence of SEQ ID NO: 155, and the HVR-L3 sequence of SEQ ID NO: 163,
11) an antibody comprising the HVR-H1 sequence of SEQ ID NO: 124, the HVR-H2 sequence of SEQ ID NO: 132, the HVR-H3 sequence of SEQ ID NO: 140, the HVR-L1 sequence of SEQ ID NO: 148, the HVR-L2 sequence of SEQ ID NO: 156, and the HVR-L3 sequence of SEQ ID NO: 164,
12) an antibody comprising the HVR-H1 sequence of SEQ ID NO: 125, the HVR-H2 sequence of SEQ ID NO: 133, the HVR-H3 sequence of SEQ ID NO: 141, the HVR-L1 sequence of SEQ ID NO: 149, the HVR-L2 sequence of SEQ ID NO: 157, and the HVR-L3 sequence of SEQ ID NO: 165, and
13) an antibody comprising the HVR-Hi sequence of SEQ ID NO: 126, the HVR-H2 sequence of SEQ ID NO: 134, the HVR-H3 sequence of SEQ ID NO: 142, the HVR-L1 sequence of SEQ ID NO: 150, the HVR-L2 sequence of SEQ ID NO: 158, and the HVR-L3 sequence of SEQ ID NO: 166.

In one aspect, the present disclosure provides an isolated humanized monoclonal antibody with pH-dependent binding that specifically binds to an epitope within a region encompassing the CUB1-EGF-CUB2 domain consisting of CUB1, EGF, and CUB2 of complement component is (C1s). In some embodiments, the epitope bound by an isolated anti-C1s antibody of the present disclosure is an epitope not located in beta domain of C1s. In some embodiments, the epitope bound by an isolated anti-C1s antibody of the present disclosure is an epitope located in alpha domain of C1s or gamma domain of C1s. In some embodiments, the epitope bound by an isolated anti-C1s antibody of the present disclosure is a linear epitope. In some embodiments, the epitope bound by an isolated anti-C1s antibody of the present disclosure is an epitope within amino acids 16-291 of the complement C1s protein, amino acids 16-172 of the complement C1s protein set forth in SEQ ID NO: 1, amino acids 16-210 of the complement C1s protein set forth in SEQ ID NO: 1, amino acids 16-111 of the complement C1s protein set forth in SEQ ID NO: 1, amino acids 112-210 of the complement C1s protein set forth in SEQ ID NO: 1, amino acids 131-172 of the complement C1s protein set forth in SEQ ID NO: 1, or amino acids 16-130 of the complement C1s protein set forth in SEQ ID NO: 1. In some embodiments, the above-described epitope of C1s is an epitope of human C1s. In some embodiments, an isolated anti-C1s antibody of the present invention can bind to both an activated C1s protein and an inactive form of C1s.

In some embodiments, the present disclosure provides an isolated anti-C1r antibody that specifically binds to an epitope within a region encompassing the CUB1-EGF-CUB2 domain consisting of CUB1, EGF, and CUB2 of complement component 1r (C1r). In some cases, the epitope bound by an isolated anti-C1r antibody of the present disclosure is a linear epitope or conformational epitope. In some embodiments, the above-described epitope of C1r is an epitope of human C1r.

In a further aspect of the invention, an anti-C1s antibody according to any of the above embodiments is a monoclonal antibody, including a chimeric, humanized or human antibody. In one embodiment, an anti-C1s antibody is an antibody fragment, e.g., a Fv, Fab, Fab′, scFv, diabody, or F(ab′)2 fragment. In another embodiment, the antibody is a full length antibody, e.g., an intact IgG1, IgG2, IgG3 or IgG4 antibody or other antibody class or isotype as defined herein.

In a further aspect, an anti-C1s antibody according to any of the above embodiments may incorporate any of the features, singly or in combination, as described in Sections 1-7 below:

### 1. Antibody Affinity

In certain embodiments, an antibody provided herein has a dissociation constant (Kd or KD) of 1 micro M or less, 100 nM or less, 10 nM or less, 1 nM or less, 0.1 nM or less, 0.01 nM or less, or 0.001 nM or less (e.g. 10-8 M or less, e.g. from 10-8 M to 10-13 M, e.g., from 10-9 M to 10-13 M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 micro l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (GE Healthcare) is performed at 25 degrees C. with immobilized antigen CM5 chips at ˜10 response units (RU). In one embodiment, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 microgram (micro g)/ml (˜0.2 micro M) before injection at a flow rate of 5 microliter (micro l)/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

In some embodiments, the binding affinity of each histidine-substituted variant of the instant invention at pH 7.4 and pH 5.8 is determined at 37 degrees C. using BIACORE® T200 instrument (GE Healthcare). Recombinant Protein A/G (Pierce) can be immobilized onto all flow cells of a CM4 sensor chip using an amine coupling kit (GE Healthcare). Antibodies and analytes can be prepared in 7(+) buffer (20 mM ACES, 150 mM NaCl, 1.2 mM CaCl2, 0.05% Tween 20, 0.005% NaN3, pH 7.4), 5(+) buffer (20 mM ACES, 150 mM NaCl, 1.2 mM CaCl2, 0.05% Tween 20, 0.005% NaN3, pH 5.8), or 5(−) buffer (20 mM ACES, 150 mM NaCl, 3 micro M CaCl2, 0.05% Tween 20, 0.005% NaN3, pH 5.8). Each antibody can be captured onto the sensor surface by protein A/G. Antibody capture levels are aimed at 200 resonance unit (RU). Native proenzyme human C1s (CompTech) or recombinant human C1s prepared can be injected at 50 nM, followed by dissociation.

Specific examples of steps of Biacore assay of the present invention are as follows.

The binding specificities of C1s CUB1-EGF-CUB2 binders are determined at 37 degrees C. using BIACORE® T200 instrument (GE Healthcare). Recombinant Protein A/G (Pierce) is immobilized onto all flow cells of a CM4 sensor chip using an amine coupling kit (GE Healthcare). Antibodies and analytes are prepared in 7(+) buffer (20 mM ACES, 150 mM NaCl, 1.2 mM CaCl2, 0.05% Tween 20, 0.005% NaN3, pH 7.4). Each antibody is captured onto the sensor surface by protein A/G. Antibody capture levels are aimed at 100 resonance unit (RU). Native proenzyme human C1s (Comptech A103) (at 50 nM as a monomer) or recombinant human C1s CCP1-CCP2-SP-His (at 100nM as a monomer) is injected, followed by dissociation. Sensor surface is regenerated each cycle with 10 mM Glycine-HCl pH 1.5. It may be determined that C1s CUB1-EGF-CUB2 binders bound to the native proenzyme human C1s, but not recombinant human C1s CCP1-CCP2-SP-His, which is a truncated protein lacking the CUB1-EGF-CUB2 domain.

The C1q displacement function of antibodies is demonstrated by a C1r2s2 capture method using BIACORE® T200 instrument (GE Healthcare) at 37 degrees C. An anti-His antibody (GE-Healthcare) is immobilized onto all flow cells of a CM4 sensor chip using an amine coupling kit (GE Healthcare). The antibodies, recombinant human C1r2s2 Flag/His tetramer and native human C1q (Comptech A099) are prepared in pH 7.4 buffer (20 mM ACES, 150 mM NaCl, 1.2 mM CaCl2, 1 mg/mL BSA (IgG-free), 1 mg/mL CMD, 0.05% Tween 20, 0.005% NaN3, pH 7.4). Recombinant human C1r2s2 Flag/His tetramer is first captured onto the sensor surface by the anti-His antibody (“hc1r2s2”). The capture levels are aimed at 200 resonance unit (RU). Native human C1q is injected at 100 nM to have a capture of 200 RU (“hc1q”), followed by antibody injection at 500 nM at 10 micro L/min for 1200 sec immediately. The sensor surface is regenerated each cycle with 10 mM Glycine-HCl (pH 1.5). For antibodies with the C1q displacement function, the response unit of Sensorgram 2 (in the presence of C1r2s2, C1q, and antibody) is lower than the response unit in Sensorgram 1 (in the presence of C1r2s2, C1q, and buffer, but in the absence of antibody) after the time point where Sensorgrams 1 and 2 cross (“time point of crossover”). The time point of crossover is identified by subtraction of the buffer response (Sensorgram 1) from the antibody (Ab) response (Sensorgram 2), and referring to the time point when the differential value changes from positive to negative.

The C1q displacement function of the antibodies is demonstrated by a C1q capture method using BIACORE® T200 instrument (GE Healthcare) at 37 degrees C. The antibodies, recombinant human C1r2s2 Flag/His tetramer and native human C1q (Comptech A099) that has been biotinylated are prepared in pH 7.4 buffer (20 mM ACES, 150 mM NaCl, 1.2 mM CaCl2, 1 mg/mL BSA (IgG-free), 1 mg/mL CMD, 0.05% Tween 20, 0.005% NaN3, pH 7.4). Biotinylated native human C1q is first captured onto one flow cell of a CAP sensor chip (GE-Healthcare). The capture levels are aimed in the range of 800 to 1000 resonance unit (RU). Recombinant human C1r2s2 Flag/His tetramer is injected at 300 nM, followed by antibody injection at 500 nM at 10 micro L/min for 180 sec. The sensor surface is regenerated each cycle with 8 M Guanidine-HCl and 1 M NaOH in 3-to-1 ratio. Antibodies with the C1q displacement function enhance the dissociation rate of C1r2s2, i.e., the curve in the presence of the antibody runs below the curve in the absence of the antibody.

To assess the blocking of C1q binding to C1r2s2 by the antibodies, blocking assay is performed at 37 degrees C. using BIACORE® T200 instrument (GE Healthcare). An anti-His antibody (GE-Healthcare) is immobilized onto all flow cells of a CM4 sensor chip using an amine coupling kit (GE Healthcare). The antibodies, recombinant human C1r2s2 Flag/His tetramer and native human C1q are prepared in pH 7.4 buffer (20 mM ACES, 150 mM NaCl, 1.2 mM CaCl2, 1 mg/mL BSA (IgG-free), 1 mg/mL CMD, 0.05% Tween 20, 0.005% NaN3, pH 7.4). Recombinant human C1r2s2 Flag/His tetramer is first captured onto the sensor surface by the anti-His antibody (“hc1r2s2”). The capture levels are aimed at 200 resonance unit (RU). The antibody variants are injected at 500 nM, followed by native human C1q injection at 100 nM (“hc1q”). The sensor surface is regenerated each cycle with 10 mM Glycine-HCl (pH 1.5). Antibodies with C1q blocking function are those which compete with C1q for binding to C1r2s2.

In some embodiments, an additional dissociation phase at pH 5.8 is integrated immediately after the dissociation phase at pH 7.4, if necessary. This dissociation rate in 5(+) buffer can be determined by processing and fitting data using Scrubber 2.0 (BioLogic Software) curve fitting software.

### 2. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE ® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for C1s and the other is for any other antigen. In certain embodiments, bispecific antibodies may bind to two different epitopes of C1s. Bispecific antibodies may also be used to localize cytotoxic agents to cells which express C1s. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting Fab” or “DAF” comprising an antigen binding site that binds to C1s as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Gln | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be analyzed to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g. for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

### (Sweeping Technology)

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. In some embodiments, the Fc region is an Fc region of human IgG1.

To enhance the reduction of plasma antigen concentration and/or improve pharmacokinetics of antibodies, amino acid residues at the site for binding to FcRn in the Fc region of IgG can be modified to enhance their uptake into cells. When an antibody with pH dependency is modified in this way, the mutant will be a “sweeping” antibody that can more strongly bind to FcRn and allow the antigen to be efficiently transferred into the endosome (where pH is acidic) and then degraded, but can itself be more efficiently recycled to the cell surface. Such a modified, “sweeping” antibody can strongly bind to FcRn at neutral pH and on the cell surface and enhance the uptake and degradation of the antigen, compared to the original (parent) antibody without the modification. (Semin Immunopathol. 2018; 40(1): 125-140).

In some aspects, the antibody comprises an Fc region that has at least one amino acid modification in the Fc region so as to enhance the reduction of plasma antigen concentration and/or improve pharmacokinetics of the antibody.

In some embodiments, the Fc region is a human Fc region that has a binding activity to an activating Fc gamma receptor is stronger than the binding activity of an Fc region of the native human IgG1. As mentioned in, e.g., WO 2013/047752, to enhance the binding activity to an activating Fc gamma receptor, one or more amino acids selected from the group consisting of amino acids at positions 221, 222, 223, 224, 225, 227, 228, 230, 231, 232, 233, 234, 235, 236, 237, 238, 239, 240, 241, 243, 244, 245, 246, 247, 249, 250, 251, 254, 255, 256, 258, 260, 262, 263, 264, 265, 266, 267, 268, 269, 270, 271, 272, 273, 274, 275, 276, 278, 279, 280, 281, 282, 283, 284, 285, 286, 288, 290, 291, 292, 293, 294, 295, 296, 297, 298, 299, 300, 301, 302, 303, 304, 305, 311, 313, 315, 317, 318, 320, 322, 323, 324, 325, 326, 327, 328, 329, 330, 331, 332, 333, 334, 335, 336, 337, 339, 376, 377, 378, 379, 380, 382, 385, 392, 396, 421, 427, 428, 429, 434, 436, and 440 (EU numbering) in the Fc region may be modified to be different from the amino acids at corresponding sites in the Fc region of the native human IgG1 which is the parent (original) antibody.

In some embodiments, the Fc region is a human Fc region that has a binding activity to an inhibitory Fc gamma receptor is stronger than to an activating Fc gamma receptor. As mentioned in, e.g., WO 2013/125667, to enhance the binding activity to an inhibitory Fc gamma receptor, one or more amino acids selected from the group consisting of amino acids at positions 244, 245, 249, 250, 251, 252, 253, 254, 255, 256, 257, 258, 260, 262, 265, 270, 272, 279, 283, 285, 286, 288, 293, 303, 305, 307, 308, 309, 311, 312, 314, 316, 317, 318, 332, 339, 340, 341, 343, 356, 360, 362, 375, 376, 377, 378, 380, 382, 385, 386, 387, 388, 389, 400, 413, 415, 423, 424, 427, 428, 430, 431, 433, 434, 435, 436, 438, 439, 440, 442, and 447 (EU numbering) in the Fc region may be modified to be different from the amino acids at corresponding sites in the Fc region of the native human IgG1.

In some embodiments, the Fc region is a human Fc region that has a binding activity to an FcRn at neutral pH is stronger than the binding activity of an Fc region of the native human IgG1. As mentioned in, e.g., WO 2011/122011, to enhance the binding activity to an FcRn at neutral pH, one or more amino acids selected from the group consisting of amino acids at positions 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, and 436 (EU numbering) in the Fc region may be modified to be different from the amino acids at corresponding sites in the Fc region of the native human IgG1.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACT1TM non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M.S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-C1s antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an anti-C1s antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-C1s antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Antibodies with pH-dependent characteristics may be obtained by using screening methods and/or mutagenesis methods e.g., as described in WO 2009/125825. The screening methods may comprise any process by which an antibody having pH-dependent binding characteristics is identified within a population of antibodies specific for a particular antigen. In certain embodiments, the screening methods may comprise measuring one or more binding parameters (e.g., KD or kd) of individual antibodies within an initial population of antibodies both at acidic pH and neutral pH. The binding parameters of the antibodies may be measured using, e.g., surface plasmon resonance, or any other analytic method that allows for the quantitative or qualitative assessment of the binding characteristics of an antibody to a particular antigen. In certain embodiments, the screening methods may comprise identifying an antibody that binds to an antigen with an acidic KD/neutral KD ratio of 2 or greater. Alternatively, the screening methods may comprise identifying an antibody that binds to an antigen with an acidic kd/neutral kd ratio of 2 or greater.

In another embodiment, the mutagenesis methods may comprise incorporating a deletion, substitution, or addition of an amino acid within the heavy and/or light chain of the antibody to enhance the pH-dependent binding of the antibody to an antigen. In certain embodiments, the mutagenesis may be carried out within one or more variable domains of the antibody, e.g., within one or more HVRs (e.g., CDRs). For example, the mutagenesis may comprise substituting an amino acid within one or more HVRs (e.g., CDRs) of the antibody with another amino acid. In certain embodiments, the mutagenesis may comprise substituting one or more amino acids in at least one HVR (e.g., CDR) of the antibody with histidine. In certain embodiments, “enhanced pH-dependent binding” means that the mutated version of the antibody exhibits a greater acidic KD/neutral KD ratio, or a greater acidic kd/neutral kd ratio, than the original “parent” (i.e., the less pH-dependent) version of the antibody prior to mutagenesis. In certain embodiments, the mutated version of the antibody has an acidic KD/neutral KD ratio of 2 or greater. Alternatively, the mutated version of the antibody has an acidic kd/neutral kd ratio of 2 or greater.

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups.

Animals (usually non-human mammals) are immunized against the antigen, immunogenic conjugates, or derivatives by combining, e.g., 100 micro g or 5 micro g of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature 256(5517):495-497 (1975). In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro.

The immunizing agent will typically include the antigenic protein or a fusion variant thereof. Generally either peripheral blood lymphocytes (PBLs) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press (1986), pp. 59-103).

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor et al. J. Immunol. 133(6):3001-3005 (1984); Brodeur et al., Monoclonal Antibody Production Techniques and Applications, Marcel Dekker, Inc., New York, pp. 51-63 (1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). Such techniques and assays are known in the art. For example, binding affinity may be determined by the Scatchard analysis of Munson, Anal. Biochem. 107(1):220-239 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

### C. Assays

Anti-C1s antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, etc.

In another aspect, competition assays may be used to identify an antibody that competes for binding to C1s with any anti-C1s antibody described herein, or identify an antibody that binds to the same epitope as any anti-C1s antibody described herein. In certain embodiments, when such a competing antibody is present in excess, it blocks (e.g., reduces) the binding of a reference antibody to C1s by at least 10%, 15%, 20%, 25%, 30%, 35%, 40%, 45%, 50%, 55%, 60%, 65%, 70%, 75% or more. In certain embodiments, such a competing antibody binds to the same epitope (e.g., a linear or a conformational epitope) that is bound by any anti-C1s antibody described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.). In certain embodiments, such a competition assays can be conducted at neutral pH condition. In some embodiments, the competition assay is tandem competition assay using, for example, Octet™ systems.

In an exemplary competition assay, immobilized C1s is incubated in a solution comprising a first labeled antibody that binds to C1s (e.g., one of those described herein) and a second unlabeled antibody that is being tested for its ability to compete with the first antibody for binding to C1s. The second antibody may be present in a hybridoma supernatant. As a control, immobilized C1s is incubated in a solution comprising the first labeled antibody but not the second unlabeled antibody. After incubation under conditions permissive for binding of the first antibody to C1s, excess unbound antibody is removed, and the amount of label associated with immobilized C1s is measured. If the amount of label associated with immobilized C1s is substantially reduced in the test sample relative to the control sample, then that indicates that the second antibody is competing with the first antibody for binding to C1s. See Harlow and Lane (1988) Antibodies: A Laboratory Manual ch. 14 (Cold Spring Harbor Laboratory, Cold Spring Harbor, N.Y.).

In another aspect, an antibody that binds to the same epitope as an anti-C1s antibody provided herein or that competes for binding to C1s with an anti-C1s antibody provided herein may be identified using sandwich assays. Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. See David & Greene, U.S. Pat No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. An antibody which simultaneously binds to C1s with an anti-C1s antibody provided herein can be determined to be an antibody that binds to a different epitope from the anti-C1s antibody. Therefore, an antibody which does not simultaneously bind to C1s with an anti-C1s antibody provided herein can be determined to be an antibody that binds to the same epitope as the anti-C1s antibody or that competes for binding to C1s with the anti-C1s antibody.

### 2. Activity Assays

In one aspect, assays are provided for identifying anti-C1s antibodies thereof having biological activity. Biological activity may include blocking the activation of the classical pathway and generation of cleavage products resulting from the activation of the said pathway, C2a, C2b, C3a, C3b, C4a, C4b, C5a and C5b. Antibodies having such biological activity in vivo and/or in vitro are also provided.

In certain embodiments, an antibody of the invention is tested for such biological activity. In some embodiments, the antibody of the invention can be evaluated for its ability to inhibit complement-mediated hemolysis of sheep red blood cells (RBC) that have been sensitized by antibodies directed against sheep RBC antigens, i.e., using an RBC assay. In some embodiments, the antibody of the invention can be evaluated for its ability to inhibit complement-mediated hemolysis of chicken red blood cells (cRBC) that have been sensitized by antibodies directed against cRBC antigens. Using human serum as a source of complement proteins, the activity of the antibody of the invention can be determined by measuring the amount of haemoglobin released by a spectrophotometric method.

RBC assay can be suitably performed using known methods such as the method disclosed in J. Vis. Exp. 2010; (37): 1923. This article describes how to conduct the 50% Haemolytic Complement (CH50) assay as the RBC lysis assay. Briefly, this assay measures the activation of the classical complement pathway, and detects the reduction, absence, or inactivity of any component of the pathway. It assesses the activity of complement components in the serum to lyse red blood cells. When an antibody is incubated with test serum, the pathway is activated and causes haemolysis. If one or more components of the classical pathway are decreased, the CH50 value is decreased. The CH50 assay is not exactly the same as the assay used in the Examples herein which rather measures % inhibition against cell lysis by complement components; however, the concept and basic set up is substantially the same as the present invention. In the present invention, in an embodiment, the RBC assay is performed as follows. Human serum is pre-incubated with the antibody of interest (e.g., for 3 hours at 37 degrees Celsius (degrees C.)). The serum is then added to an equal volume of sensitized sheep red blood cells and incubated (e.g., for one hour at 37 degrees C.) to allow for lysis of the red blood cells. The reaction is then stopped. The mixture is centrifuged to pellet unlysed cells, and the supernatant is withdrawn, and absorbance (OD) at 415 nm, from which OD at 630 nm is subtracted, is used to analyze the release of hemoglobin. To calculate the percentage inhibition of red blood cell lysis, 0% inhibition is set as the condition where no antibody (buffer only) is added, and 100% inhibition is set as the condition where EDTA is added at a final concentration of 5 mM (see, e.g., Example 7). When the antibody shows a percentage inhibition of red blood cell lysis, this means that the antibody has a neutralizing activity for human serum complement, e.g., an activity to inhibit the interaction between C1q and C1r2s2 complex.

Thus, RBC assay can be used to evaluate a neutralizing activity for human serum complement of an antibody of the present invention, in order to assess the activity to inhibit the interaction between C1q and C1r2s2 complex. In an embodiment, the present invention provides an isolated antibody that inhibits the interaction between C1q and C1r2s2 complex, where the antibody has a neutralizing activity for human serum complement of at least 70% in RBC assay.

### D. Immunoconjugates

The invention also provides immunoconjugates comprising an anti-C1s antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, saponaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include 211At, 131I, 125I, 90Y, 186Re, 188Re, 153Sm, 212Bi, 32P, 212Pb and radioactive isotopes of Lu. When the radioconjugate is used for detection, it may comprise a radioactive atom for scintigraphic studies, for example Tc-99m or 123I, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., USA).

### E. Methods and Compositions for Diagnostics and Detection

In certain embodiments, any of the anti-C1s antibodies provided herein is useful for detecting the presence of C1s in a biological sample. The term “detecting” as used herein encompasses quantitative or qualitative detection. In certain embodiments, a biological sample comprises a cell or tissue, such as serum, whole blood, plasma, biopsy sample, tissue sample, cell suspension, saliva, sputum, oral fluid, cerebrospinal fluid, amniotic fluid, ascites fluid, milk, colostrum, mammary gland secretion, lymph, urine, sweat, lacrimal fluid, gastric fluid, synovial fluid, peritoneal fluid, ocular lens fluid or mucus.

In one embodiment, an anti-C1s antibody for use in a method of diagnosis or detection is provided. In a further aspect, a method of detecting the presence of C1s in a biological sample is provided. In certain embodiments, the method comprises contacting the biological sample with an anti-C1s antibody as described herein under conditions permissive for binding of the anti-C1s antibody to C1s, and detecting whether a complex is formed between the anti-C1s antibody and C1s. Such method may be an in vitro or in vivo method. In one embodiment, an anti-C1s antibody is used to select subjects eligible for therapy with an anti-C1s antibody, e.g. where C1s is a biomarker for selection of patients.

Exemplary disorders that may be diagnosed using an antibody of the invention include, but are not limited to, age-related macular degeneration, Alzheimer's disease, amyotrophic lateral sclerosis, anaphylaxis, argyrophilic grain dementia, arthritis (e.g., rheumatoid arthritis), asthma, atherosclerosis, atypical hemolytic uremic syndrome, autoimmune diseases, Barraquer-Simons syndrome, Behcet's disease, British type amyloid angiopathy, bullous pemphigoid, Buerger's disease, C1q nephropathy, cancer, catastrophic antiphospholipid syndrome, cerebral amyloid angiopathy, cold agglutinin disease, corticobasal degeneration, Creutzfeldt-Jakob disease, Crohn's disease, cryoglobulinemic vasculitis, dementia pugilistica, dementia with Lewy Bodies (DLB), diffuse neurofibrillary tangles with calcification, Discoid lupus erythematosus, Down's syndrome, focal segmental glomerulosclerosis, formal thought disorder, frontotemporal dementia (FTD), frontotemporal dementia with parkinsonism linked to chromosome 17, frontotemporal lobar degeneration, Gerstmann-Straussler-Scheinker disease, Guillain-Barre syndrome, Hallervorden-Spatz disease, hemolytic-uremic syndrome, hereditary angioedema, hypophosphastasis, idiopathic pneumonia syndrome, immune complex diseases, inclusion body myositis, infectious disease (e.g., disease caused by bacterial (e.g., Neisseria meningitidis or Streptococcus) viral (e.g., human immunodeficiency virus (HIV)), or other infectious agents), inflammatory disease, ischemia/reperfusion injury, mild cognitive impairment, immunothrombocytopenic purpura (ITP), molybdenum cofactor deficiency (MoCD) type A, membranoproliferative glomerulonephritis (MPGN) I, membranoproliferative glomerulonephritis (MPGN) II (dense deposit disease), membranous nephritis, multi-infarct dementia, lupus (e.g., systemic lupus erythematosus (SLE)), glomerulonephritis, Kawasaki disease, multifocal motor neuropathy, multiple sclerosis, multiple system atrophy, myasthenia gravis, myocardial infarction, myotonic dystrophy, neuromyelitis optica, Niemann-Pick disease type C, non-Guamanian motor neuron disease with neurofibrillary tangles, Parkinson's disease, Parkinson's disease with dementia, paroxysmal nocturnal hemoglobinuria, Pemphigus vulgaris, Pick's disease, postencephalitic parkinsonism, polymyositis, prion protein cerebral amyloid angiopathy, progressive subcortical gliosis, progressive supranuclear palsy, psoriasis, sepsis, Shiga-toxin E. coli (STEC)-HuS, spinal muscular atrophy, stroke, subacute sclerosing panencephalitis, Tangle only dementia, transplant rejection, vasculitis (e.g., ANCA associated vasculitis), Wegner's granulomatosis, sickle cell disease, cryoglobulinemia, mixed cryoglobulinemia, essential mixed cryoglobulinemia, Type II mixed cryoglobulinemia, Type III mixed cryoglobulinemia, nephritis, drug-induced thrombocytopenia, lupus nephritis, bullous pemphigoid, Epidermolysis bullosa acquisita, delayed hemolytic transfusion reaction, hypocomplementemic urticarial vasculitis syndrome, pseudophakic bullous keratopathy, and platelet refractoriness.

In certain embodiments, labeled anti-C1s antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 125I, 3H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

Pharmaceutical formulations of an anti-C1s antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide the formulation which is used for combination therapy, Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the anti-C1s antibodies provided herein may be used in therapeutic methods.

In one aspect, an anti-C1s antibody for use as a medicament is provided. In further aspects, an anti-C1s antibody for use in treating a complement-mediated disease or disorder is provided. In certain embodiments, an anti-C1s antibody for use in a method of treatment is provided. In certain embodiments, the invention provides an anti-C1s antibody for use in a method of treating an individual having a complement-mediated disease or disorder comprising administering to the individual an effective amount of the anti-C1s antibody. In one such embodiment, the method further comprises administering to the individual an effective amount of at least one additional therapeutic agent.

In further embodiments, the invention provides an anti-C1s antibody for use in treating a complement-mediated disease or disorder. In further embodiments, anti-C1s antibodies of the present invention may be for use in enhancing the clearance of C1s from plasma. In further embodiments, anti-C1s antibodies of the present invention may be for use in enhancing the clearance of C1r2s2 from plasma. In further embodiments, anti-C1s antibodies of the present invention may be for use in enhancing the clearance of C1r2s2 from plasma not but C1q from plasma. In some cases, the antibody inhibits a component of the classical complement pathway; in some cases, the classical complement pathway component is C1s. In certain embodiments, the invention provides an anti-C1s antibody for use in a method of treating a complement-mediated disease or disorder. In certain embodiments, the invention provides an anti-C1s antibody for use in a method of enhancing the clearance of C1s from plasma. In certain embodiments, the invention provides an anti-C1s antibody for use in a method of enhancing the clearance of C1r2s2 from plasma. In certain embodiments, the invention provides an anti-C1s antibody for use in a method of enhancing the clearance of C1r2s2 from plasma not but C1q from plasma. In certain embodiments, the invention provides an anti-C1s antibody for use in a method of inhibiting a component of the classical complement pathway; in some cases, the classical complement pathway component is C1s. An “individual” according to any of the above embodiments is preferably a human.

In one aspect, the present disclosure provides a method of modulating complement activation. In some embodiments the method inhibits complement activation, for example to reduce production of C4b2a. In some embodiments, the present disclosure provides a method of modulating complement activation in an individual having a complement-mediated disease or disorder, the method comprising administering to the individual an anti-C1s antibody of the present disclosure or a pharmaceutical composition of the present disclosure, wherein the pharmaceutical composition comprises an anti-C1s antibody of the present disclosure. In some embodiments such a method inhibits complement activation. In some embodiments, the individual is a mammal. In some embodiments, the individual is a human. Administration can be by any route known to those skilled in the art, including those disclosed herein. In some embodiments, administration is intravenous or subcutaneous. In some embodiments, administration is intrathecal.

A complement-mediated disease or disorder is a disorder characterized by an abnormal amount of complement C1s or an abnormal level of complement C1s proteolytic activity in a cell, a tissue, or a fluid of an individual.

In some cases, a complement-mediated disease or disorder is characterized by the presence in a cell, a tissue, or a fluid of an elevated (higher than normal) amount of C1s or of an elevated level of complement C1s activity. For example, in some cases, a complement-mediated disease or disorder is characterized by the presence in brain tissue and/or cerebrospinal fluid of an elevated amount and/or an elevated activity of C1s. A “higher than normal” amount of C1s in a cell, a tissue, or a fluid indicates that the amount of C1s in the cell, tissue or fluid is higher than a normal, control level, e.g., higher than a normal, control level for an individual or population of individuals of the same age group. A “higher than normal” level of C1s activity in a cell, a tissue, or a fluid indicates that the proteolytic cleavage effected by C1s in the cell, tissue or fluid is higher than a normal, control level, e.g., higher than a normal, control level for an individual or population of individuals of the same age group. In some cases, an individual having a complement-mediated disease or disorder exhibits one or more additional symptoms of such a disease or disorder.

In other cases, a complement-mediated disease or disorder is characterized by the presence in a cell, a tissue, or a fluid of a lower than normal amount of C1s or of a lower level of complement C1s activity. For example, in some cases, a complement-mediated disease or disorder is characterized by the presence in brain tissue and/or cerebrospinal fluid of a lower amount and/or a lower activity of C1s. A “lower than normal” amount of C1s in a cell, a tissue, or a fluid indicates that the amount of C1s in the cell, tissue or fluid is lower than a normal, control level, e.g., lower than a normal, control level for an individual or population of individuals of the same age group. A “lower than normal” level of C1s activity in a cell, a tissue, or a fluid indicates that the proteolytic cleavage effected by C1s in the cell, tissue or fluid is lower than a normal, control level, e.g., lower than a normal, control level for an individual or population of individuals of the same age group. In some cases, an individual having a complement-mediated disease or disorder exhibits one or more additional symptoms of such a disease or disorder.

A complement-mediated disease or disorder is a disease or disorder in which the amount or activity of complement C1s is such that it causes a disease or disorder in an individual. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In some embodiments, the complement-mediated disease or disorder is an autoimmune disease. In some embodiments, the complement-mediated disease or disorder is cancer. In some embodiments, the complement-mediated disease or disorder is an infectious disease. In some embodiments, the complement-mediated disease or disorder is an inflammatory disease. In some embodiments, the complement-mediated disease or disorder is a hematological disease. In some embodiments, the complement-mediated disease or disorder is an ischemia-reperfusion injury. In some embodiments, the complement-mediated disease or disorder is an ocular disease. In some embodiments, the complement-mediated disease or disorder is a renal disease. In some embodiments, the complement-mediated disease or disorder is transplant rejection. In some embodiments, the complement-mediated disease or disorder is antibody-mediated transplant rejection. In some embodiments, the complement-mediated disease or disorder is a vascular disease. In some embodiments, the complement-mediated disease or disorder is a vasculitis disorder. In some embodiments, the complement-mediated disease or disorder is a neurodegenerative disease or disorder. In some embodiments, the complement-mediated disease is a neurodegenerative disease. In some embodiments, the complement-mediated disorder is a neurodegenerative disorder. In some embodiments, the complement-mediated disease or disorder is a tauopathy.

Examples of a complement-mediated disease or disorder include, but are not limited to, age-related macular degeneration, Alzheimer's disease, amyotrophic lateral sclerosis, anaphylaxis, argyrophilic grain dementia, arthritis (e.g., rheumatoid arthritis), asthma, atherosclerosis, atypical hemolytic uremic syndrome, autoimmune diseases, Barraquer-Simons syndrome, Behcet's disease, British type amyloid angiopathy, bullous pemphigoid, Buerger's disease, C1q nephropathy, cancer, catastrophic antiphospholipid syndrome, cerebral amyloid angiopathy, cold agglutinin disease, corticobasal degeneration, Creutzfeldt-Jakob disease, Crohn's disease, cryoglobulinemic vasculitis, dementia pugilistica, dementia with Lewy Bodies (DLB), diffuse neurofibrillary tangles with calcification, Discoid lupus erythematosus, Down's syndrome, focal segmental glomerulosclerosis, formal thought disorder, frontotemporal dementia (FTD), frontotemporal dementia with parkinsonism linked to chromosome 17, frontotemporal lobar degeneration, Gerstmann-Straussler-Scheinker disease, Guillain-Barre syndrome, Hallervorden-Spatz disease, hemolytic-uremic syndrome, hereditary angioedema, hypophosphastasis, idiopathic pneumonia syndrome, immune complex diseases, inclusion body myositis, infectious disease (e.g., disease caused by bacterial (e.g., Neisseria meningitidis or Streptococcus) viral (e.g., human immunodeficiency virus (HIV)), or other infectious agents), inflammatory disease, ischemia/reperfusion injury, mild cognitive impairment, immunothrombocytopenic purpura (ITP), molybdenum cofactor deficiency (MoCD) type A, membranoproliferative glomerulonephritis (MPGN) I, membranoproliferative glomerulonephritis (MPGN) II (dense deposit disease), membranous nephritis, multi-infarct dementia, lupus (e.g., systemic lupus erythematosus (SLE)), glomerulonephritis, Kawasaki disease, multifocal motor neuropathy, multiple sclerosis, multiple system atrophy, myasthenia gravis, myocardial infarction, myotonic dystrophy, neuromyelitis optica, Niemann-Pick disease type C, non-Guamanian motor neuron disease with neurofibrillary tangles, Parkinson's disease, Parkinson's disease with dementia, paroxysmal nocturnal hemoglobinuria, Pemphigus vulgaris, Pick's disease, postencephalitic parkinsonism, polymyositis, prion protein cerebral amyloid angiopathy, progressive subcortical gliosis, progressive supranuclear palsy, psoriasis, sepsis, Shiga-toxin E. coli (STEC)-HuS, spinal muscular atrophy, stroke, subacute sclerosing panencephalitis, Tangle only dementia, transplant rejection, vasculitis (e.g., ANCA associated vasculitis), Wegner's granulomatosis, sickle cell disease, cryoglobulinemia, mixed cryoglobulinemia, essential mixed cryoglobulinemia, Type II mixed cryoglobulinemia, Type III mixed cryoglobulinemia, nephritis, drug-induced thrombocytopenia, lupus nephritis, bullous pemphigoid, Epidermolysis bullosa acquisita, delayed hemolytic transfusion reaction, hypocomplementemic urticarial vasculitis syndrome, pseudophakic bullous keratopathy, and platelet refractoriness.

Alzheimer's disease and certain forms of Frontotemporal dementia (Pick's disease, sporadic Frontotemporal dementia and Frontotemporal dementia with Parkinsonism linked to chromosome 17) are the most common forms of tauopathy. In accordance with this, the present invention relates to any method as described above, wherein the tauopathy is Alzheimer's, Pick's disease, sporadic Frontotemporal dementia and Frontotemporal dementia with Parkinsonism linked to chromosome 17. Other tauopathies include, but are not limited to, Progressive supranuclear palsy (PSP), Corticobasal degeneration (CBD) and Subacute sclerosing panencephalitis.

A neurodegenerative tauopathy includes Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia, ischemic stroke, chronic traumatic encephalopathy (CTE), traumatic brain injury (TBI), and stroke.

The present disclosure also provides methods of treating a synucleinopathy, e.g., Parkinson's disease (PD); dementia with Lewy Bodies (DLB); multiple system atrophy (MSA); etc. For example, PD with dementia (PDD) can be treated with a method of the present disclosure.

In some embodiments, the complement-mediated disease or disorder comprises Alzheimer's disease. In some embodiments, the complement-mediated disease or disorder comprises Parkinson's disease. In some embodiments, the complement-mediated disease or disorder comprises transplant rejection. In some embodiments, the complement-mediated disease or disorder is antibody-mediated transplant rejection.

In some embodiments, an anti-C1s antibody of the present disclosure prevents or delays the onset of at least one symptom of a complement-mediated disease or disorder in an individual. In some embodiment, an anti-C1s antibody of the present disclosure reduces or eliminates at least one symptom of a complement-mediated disease or disorder in an individual. Examples of symptoms include, but are not limited to, symptoms associated with autoimmune disease, cancer, hematological disease, infectious disease, inflammatory disease, ischemia-reperfusion injury, neurodegenerative disease, neurodegenerative disorder, renal disease, transplant rejection, ocular disease, vascular disease, or a vasculitis disorder. The symptom can be a neurological symptom, for example, impaired cognitive function, memory impairment, loss of motor function, etc. The symptom can also be the activity of C1s protein in a cell, tissue, or fluid of an individual. The symptom can also be the extent of complement activation in a cell, tissue, or fluid of an individual.

In some embodiments, administering an anti-C1s antibody of the present disclosure to an individual modulates complement activation in a cell, tissue, or fluid of an individual. In some embodiments, administration of an anti-C1s antibody of the present disclosure to an individual inhibits complement activation in a cell, tissue, or fluid of an individual. For example, in some embodiments, an anti-C1s antibody of the present disclosure, when administered in one or more doses as monotherapy or in combination therapy to an individual having a complement-mediated disease or disorder, inhibits complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the anti-C1s antibody.

In some embodiments, an anti-C1s antibody of the present disclosure reduces C3 deposition onto red blood cells; for example, in some embodiments, an anti-C1s antibody of the present disclosure reduces deposition of C3b, iC3b, etc., onto RBCs. In some embodiments, an anti-C1s antibody of the present disclosure inhibits complement-mediated red blood cell lysis.

In some embodiments, an anti-C1s antibody of the present disclosure reduces C3 deposition onto platelets; for example, in some embodiments, an anti-C1s antibody of the present disclosure reduces deposition of C3b, iC3b, etc., onto platelets.

In some embodiments, administering an anti-C1s antibody of the present disclosure results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (l) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction of C3 deposition on red blood cells (e.g., a reduction of deposition of C3b, iC3b, etc., onto RBCs); and (ad) a reduction in C3 deposition on platelets (e.g., a reduction of deposition of C3b, iC3b, etc., onto platelets); and (ae) a reduction of anaphylatoxin toxin production; (af) a reduction in autoantibody mediated blister formation; (ag) a reduction in autoantibody induced pruritis; (ah) a reduction in autoantibody induced erythematosus; (ai) a reduction in autoantibody mediated skin erosion; (aj) a reduction in red blood cell destruction due to transfusion reactions; (ak) a reduction in red blood cell lysis due to alloantibodies; (al) a reduction in hemolysis due to transfusion reactions; (am) a reduction in allo-antibody mediated platelet lysis; (an) a reduction in platelet lysis due to transfusion reactions; (ao) a reduction in mast cell activation; (ap) a reduction in mast cell histamine release; (aq) a reduction in vascular permeability; (ar) a reduction in edema; (as) a reduction in complement deposition on transplant graft endothelium; (at) a reduction of anaphylatoxin generation in transplant graft endothelium; (au) a reduction in the separation of the dermal-epidermal junction; (av) a reduction in the generation of anaphylatoxins in the dermal-epidermal junction; (aw) a reduction in alloantibody mediated complement activation in transplant graft endothelium; (ax) a reduction in antibody mediated loss of the neuromuscular junction; (ay) a reduction in complement activation at the neuromuscular junction; (az) a reduction in anaphylatoxin generation at the neuromuscular junction; (ba) a reduction in complement deposition at the neuromuscular junction; (bb) a reduction in paralysis; (be) a reduction in numbness; (bd) increased bladder control; (be) increased bowel control; (bf) a reduction in mortality associated with autoantibodies; and (bg) a reduction in morbidity associated with autoantibodies.

In some embodiments, an anti-C1s antibody of the present disclosure, when administered in one or more doses as monotherapy or in combination therapy to an individual having a complement-mediated disease or disorder, is effect to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (l) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; compared to the level or degree of the outcome in the individual before treatment with the anti-C1s antibody.

In some embodiments, an anti-C1s antibody of the present disclosure, when administered in one or more doses as monotherapy or in combination therapy to an individual having a complement-mediated disease or disorder, is effect to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation; f) clotting; g) kidney function; and h) hematocrit (red blood cell count), compared to the level or degree of the outcome in the individual before treatment with the anti-C1s antibody.

In some embodiments, administering an anti-C1s antibody of the present disclosure to an individual reduces complement activation in the individual. For example, in some embodiments, an anti-C1s antibody of the present disclosure, when administered in one or more doses as monotherapy or in combination therapy to an individual having a complement-mediated disease or disorder, reduces complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the anti-C1s antibody.

In some embodiments, administering an anti-C1s antibody of the present disclosure improves cognitive function in the individual. For example, in some embodiments, an anti-C1s antibody of the present disclosure, when administered in one or more doses as monotherapy or in combination therapy to an individual having a complement-mediated disease or disorder, improves cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the cognitive function in the individual before treatment with the anti-C1s antibody.

In some embodiments, administering an anti-C1s antibody of the present disclosure reduces the rate of decline in cognitive function in the individual. For example, in some embodiments, an anti-C1s antibody of the present disclosure, when administered in one or more doses as monotherapy or in combination therapy to an individual having a complement-mediated disease or disorder, reduces the rate of decline of cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the rate of decline in cognitive function in the individual before treatment with the anti-C1s antibody.

In some embodiments, administering an anti-C1s antibody of the present disclosure to an individual reduces neuron loss in the individual. For example, in some embodiments, an anti-C1s antibody of the present disclosure, when administered in one or more doses as monotherapy or in combination therapy to an individual having a complement-mediated disease or disorder, reduces neuron loss in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to neuron loss in the individual before treatment with the anti-C1s antibody.

In some embodiments, administering an anti-C1s antibody of the present disclosure to an individual reduces phospho-Tau levels in the individual. For example, in some embodiments, an anti-C1s antibody of the present disclosure, when administered in one or more doses as monotherapy or in combination therapy to an individual having a complement-mediated disease or disorder, reduces phospho-Tau in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the phospho-Tau level in the individual before treatment with the anti-C1s antibody.

In some embodiments, administering an anti-C1s antibody of the present disclosure to an individual reduces glial cell activation in the individual. For example, in some embodiments, an anti-C1s antibody of the present disclosure, when administered in one or more doses as monotherapy or in combination therapy to an individual having a complement-mediated disease or disorder, reduces glial activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to glial cell activation in the individual before treatment with the anti-C1s antibody. In some embodiments, the glial cells are astrocytes or microglia.

In some embodiments, administering an anti-C1s antibody of the present disclosure to an individual reduces lymphocyte infiltration in the individual. For example, in some embodiments, an anti-C1s antibody of the present disclosure, when administered in one or more doses as monotherapy or in combination therapy to an individual having a complement-mediated disease or disorder, reduces lymphocyte infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to lymphocyte infiltration in the individual before treatment with the anti-C1s antibody.

In some embodiments, administering an anti-C1s antibody of the present disclosure to an individual reduces macrophage infiltration in the individual. For example, in some embodiments, an anti-C1s antibody of the present disclosure, when administered in one or more doses as monotherapy or in combination therapy to an individual having a complement-mediated disease or disorder, reduces macrophage infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to macrophage infiltration in the individual before treatment with the anti-C1s antibody.

In some embodiments, administering an anti-C1s antibody of the present disclosure to an individual reduces antibody deposition in the individual. For example, in some embodiments, an anti-C1s antibody of the present disclosure, when administered in one or more doses as monotherapy or in combination therapy to an individual having a complement-mediated disease or disorder, reduces antibody deposition in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to antibody deposition in the individual before treatment with the anti-C1s antibody.

In some embodiments, administering an anti-C1s antibody of the present disclosure to an individual reduces anaphylatoxin (e.g., C3a, C4a, C5a) production in an individual. For example, in some embodiments, an anti-C1s antibody of the present disclosure, when administered in one or more doses as monotherapy or in combination therapy to an individual having a complement-mediated disease or disorder, reduces anaphylatoxin production in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of anaphylatoxin production in the individual before treatment with the anti-C1s antibody.

In some embodiments, the present disclosure provides for use of an anti-C1s antibody of the present disclosure or a pharmaceutical composition comprising an anti-C1s antibody of the present disclosure and a pharmaceutically acceptable excipient to treat an individual having a complement-mediated disease or disorder. In some embodiments, the present disclosure provides for use of an anti-C1s antibody of the present disclosure to treat an individual having a complement-mediated disease or disorder. In some embodiments, the present disclosure provides for use of a pharmaceutical composition comprising an anti-C1s antibody of the present disclosure and a pharmaceutically acceptable excipient to treat an individual having a complement-mediated disease or disorder.

In some embodiments, the present disclosure provides for use of an anti-C1s antibody of the present disclosure in the manufacture of a medicament for the treatment of an individual having a complement-mediated disease or disorder.

In some embodiments, the present disclosure provides for use of an anti-C1s antibody of the present disclosure or a pharmaceutical composition comprising an anti-C1s antibody of the present disclosure and a pharmaceutically acceptable excipient to inhibit complement activation. In some embodiments, the present disclosure provides for use of an anti-C1s antibody of the present disclosure or a pharmaceutical composition comprising an anti-C1s antibody of the present disclosure and a pharmaceutically acceptable excipient to inhibit complement activation in an individual having a complement-mediated disease or disorder. In some embodiments, the present disclosure provides for use of an anti-C1s antibody of the present disclosure to inhibit complement activation in an individual having a complement-mediated disease or disorder. In some embodiments, the present disclosure provides for use of a pharmaceutical composition comprising an anti-C1s antibody of the present disclosure and a pharmaceutically acceptable excipient to inhibit complement activation in an individual having a complement-mediated disease or disorder.

In some embodiments, the present disclosure provides for use of an anti-C1s antibody of the present disclosure in the manufacture of a medicament for modulating complement activation. In some embodiments, the medicament inhibits complement activation. In some embodiments, the medicament inhibits complement activation in an individual having a complement-mediated disease or disorder.

In some embodiments, the present disclosure provides for an anti-C1s antibody of the present disclosure or a pharmaceutical composition comprising an anti-C1s antibody of the present disclosure and a pharmaceutically acceptable excipient for use in medical therapy. In some embodiments, the present disclosure provides for an anti-C1s antibody of the present disclosure for use in medical therapy. In some embodiments, the present disclosure provides for a pharmaceutical composition comprising an anti-C1s antibody of the present disclosure and a pharmaceutically acceptable excipient for use in medical therapy.

In some embodiments, the present disclosure provides for an anti-C1s antibody of the present disclosure or a pharmaceutical composition comprising an anti-C1s antibody of the present disclosure and a pharmaceutically acceptable excipient for treating an individual having a complement-mediated disease or disorder. In some embodiments, the present disclosure provides for an anti-C1s antibody of the present disclosure for treating an individual having a complement-mediated disease or disorder. In some embodiments, the present disclosure provides for a pharmaceutical composition comprising an anti-C1s antibody of the present disclosure and a pharmaceutically acceptable excipient for treating an individual having a complement-mediated disease or disorder.

In some embodiments, the present disclosure provides for an anti-C1s antibody of the present disclosure or a pharmaceutical composition comprising an anti-C1s antibody of the present disclosure and a pharmaceutically acceptable excipient for modulating complement activation. In some embodiments, the present disclosure provides for an anti-C1s antibody of the present disclosure for modulating complement activation. In some embodiments, the present disclosure provides for a pharmaceutical composition comprising an anti-C1s antibody of the present disclosure and a pharmaceutically acceptable excipient for modulating complement activation. In some embodiments, the anti-C1s antibody inhibits complement activation.

In a further aspect, the invention provides for the use of an anti-C1s antibody in the manufacture or preparation of a medicament. In one embodiment, the medicament is for treatment of a complement-mediated disease or disorder. In a further embodiment, the medicament is for use in a method of treating a complement-mediated disease or disorder comprising administering to an individual having a complement-mediated disease or disorder an effective amount of the medicament. In one such embodiment, the method further comprises administering to the individual an effective amount of at least one additional therapeutic agent, e.g., as described below. In a further embodiment, the medicament is for use in enhancing the clearance of (or removing) C1s from plasma. In a further embodiment, the medicament is for use in enhancing the clearance of (or removing) C1r2s2 from plasma. In a further embodiment, the medicament is for use in enhancing the clearance of (or removing) C1r2s2 from plasma not but C1q from plasma. In a further embodiment, the medicament is for use in inhibiting a component of the classical complement pathway; in some cases, the classical complement pathway component is C1s

In a further embodiment, the medicament is for use in a method of treating in an individual having a complement-mediated disease or disorder comprising administering to the individual an amount effective of the medicament. An “individual” according to any of the above embodiments may be a human.

In a further aspect, the invention provides a method for treating a complement-mediated disease or disorder. In one embodiment, the method comprises administering to an individual having such a complement-mediated disease or disorder an effective amount of an anti-C1s antibody. In one such embodiment, the method further comprises administering to the individual an effective amount of at least one additional therapeutic agent, as described below. An “individual” according to any of the above embodiments may be a human.

In a further aspect, the invention provides a method for enhancing the clearance of (or removing) C1s from plasma in an individual. In a further aspect, the invention provides a method for enhancing the clearance of (or removing) C1r2s2 from plasma in an individual. In a further aspect, the invention provides a method for enhancing the clearance of (or removing) C1r2s2from plasma not but C1q from plasma in an individual In some cases, the invention provides a method for inhibiting a component of the classical complement pathway in an individual; in some cases, the classical complement pathway component is C1s. In one embodiment, an “individual” is a human.

In a further aspect, the invention provides pharmaceutical formulations comprising any of the anti-C1s antibodies provided herein, e.g., for use in any of the above therapeutic methods. In one embodiment, a pharmaceutical formulation comprises any of the anti-C1s antibodies provided herein and a pharmaceutically acceptable carrier. In another embodiment, a pharmaceutical formulation comprises any of the anti-C1s antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-C1s antibody and administration of an additional therapeutic agent occur within about one month, or within about one, two or three weeks, or within about one, two, three, four, five, or six days, of each other. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 micro g/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using an immunoconjugate of the invention in place of or in addition to an anti-C1s antibody.

### H. Articles of Manufacture

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-C1s antibody.

### III. EXAMPLES

The following are examples of methods and compositions of the invention. It is understood that various other embodiments may be practiced, given the general description provided above.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Example 1

### Expression and Purification of Proteins

### Example 1.1

### Expression and Purification of Recombinant Human C1r2s2 Flag/His Tetramer

The sequences used for expression and purification are: human C1s (NCBI Reference Sequence: NP_958850.1) with C-terminus GGGGS linker and 8× Histidine tag (SEQ ID NO: 7) and human C1r (NCBI Reference Sequence: NP_001724.3) with C-terminus GGGGS linker and FLAG tag. The human C1r sequence has R463Q S654A mutations (Kardos et. al. J Immunol. 2001 Nov. 1; 167(9):5202-8) (SEQ ID NO: 8). For the expression of recombinant human C1r2s2 Flag/His tetramer, human C1s-His and human C1r-Flag were co-expressed transiently using FreeStyle293-F cell line (Thermo Fisher). Conditioned media expressing recombinant human C1r2s2 Flag/His tetramer was applied to anti-Flag M2 affinity resin (Sigma) and eluted with Flag peptide (Sigma). Fractions containing recombinant human C1r2s2 Flag/His tetramer were subjected to an IMAC column (GE Healthcare) and eluted with imidazole gradient. Eluted fractions containing recombinant human C1r2s2 Flag/His tetramer were collected, concentrated and subsequently subjected to a Superdex 200 gel filtration column (GE Healthcare) equilibrated with 1×TBS, 2 mM CaC12 buffer. Fractions containing recombinant human C1r2s2 Flag/His tetramer were then pooled, concentrated, and stored at −80 degrees C.

### Example 1.2

### Expression and Purification of Recombinant Cyno C1r2s2 His/Flag Tetramer

The sequences used for expression and purification are: cynomolgus (cyno) C1s with C-terminus GGGGS linker and FLAG tag (SEQ ID NO: 9) and cyno C1r with C-terminus GGGGS linker and 8× Histidine tag. The cyno C1r sequence has R463Q S654A mutations (SEQ ID NO: 10). For the expression of recombinant cyno C1r2s2 His/Flag tetramer, cyno C1s-Flag and cyno C1r-His were co-expressed transiently using FreeStyle293-F cell line (Thermo Fisher). Conditioned media expressing recombinant cyno C1r2s2 His/Flag tetramer was applied to anti-Flag M2 affinity resin (Sigma) and eluted with Flag peptide (Sigma). Fractions containing recombinant cyno C1r2s2 His/Flag tetramer were subjected to an IMAC column (GE Healthcare) and eluted with imidazole gradient. Eluted fractions containing recombinant cyno C1r2s2 His/Flag tetramer were collected, concentrated and subsequently subjected to a Superdex 200 gel filtration column (GE Healthcare) equilibrated with 1×TBS, 2 mM CaCl2 buffer. Fractions containing recombinant cyno C1r2s2 His/Flag tetramer were then pooled, concentrated, and stored at −80 degrees C.

### EXAMPLE 1.3

### Expression and Purification of Recombinant Human C1s CCP1-CCP2-SP-His

The sequences used for expression and purification are: human C1s CCP1-CCP2-SP M292-D688 sequence (NCBI Reference Sequence: NP_958850.1) which has an N-terminus CAMPATH-1H signal sequence: MGWSCIILFLVATATGVHS (SEQ ID NO: 11). Human C1s CCP1-CCP2-SP has C-terminus 8× Histidine tag linked with GGGGS linker (SEQ ID NO: 12). The recombinant human C1s CCP1-CCP2-SP with His-tag on C-terminus was expressed transiently using FreeStyle293-F cell line (Thermo Fisher). Conditioned media expressing recombinant human C1s CCP1-CCP2-SP-His was applied to a HisTrap excel column (GE Healthcare) and eluted with imidazole gradient. Fractions containing recombinant human C1s CCP1-CCP2-SP-His protein were collected and subsequently subjected to a Superdex 200 gel filtration column (GE Healthcare) equilibrated with 1×TBS. Fractions containing recombinant human C1s CCP1-CCP2-SP-His protein were then pooled, concentrated, and stored at −80 degrees C.

### Example 1.4

### Expression and Purification of Recombinant Human C1s-Flag

The sequences used for expression and purification are: human C1s (NCBI Reference Sequence: NP_958850.1) with C-terminus GGGGS linker and Flag-tag (SEQ ID NO: 13). Recombinant human C1s-Flag was expressed transiently using Expi 293F cells (Thermo Fisher). Conditioned media expressing recombinant human C1s-Flag was applied to a column packed with anti-Flag M2 affinity resin (Sigma) and eluted with Flag peptide (Sigma) containing 1×TBS buffer. Fractions containing recombinant human C1s-Flag were collected, concentrated and subsequently subjected to a Superdex 200 gel filtration column (GE Healthcare) equilibrated with 1×TBS buffer. Fractions containing recombinant human C1s-Flag were then pooled, concentrated, and stored at −80 degrees C.

### Example 1.5

### Expression and Purification of Recombinant Cyno C1s-Flag

The sequences used for expression and purification are: cyno C1s with C-terminus GGGGS linker and Flag-tag (SEQ ID NO: 9). Recombinant cyno C1s-Flag was expressed transiently using FreeStyle293-F cell line (Thermo Fisher). Conditioned media expressing recombinant cyno C1s-Flag was applied to a column packed with anti-Flag M2 affinity resin (Sigma) and eluted with Flag peptide (Sigma) containing 1×TBS buffer. Fractions containing recombinant cyno C1s-Flag were collected, concentrated and subsequently subjected to a Superdex 200 gel filtration column (GE Healthcare) equilibrated with 1×TBS buffer. Fractions containing recombinant cyno C1s-Flag were then pooled, concentrated, and stored at −80 degrees C.

### Example 1.6

### Expression and Purification of Truncated Human C1s M1 to V173+N174Q-Flag

The sequences used for expression and purification are amino acids M1 to V173 of human C1s (NCBI Reference Sequence: NP_958850.1). The mutation of N174Q was added to follow the construct described in Tsai et. al. (Mol Immunol. 1997 December; 34(18):1273-80). GGGGS linker and Flag-tag (SEQ ID NO: 13) were added to the C-terminus. Recombinant human C1s M1 to V173+N174Q-Flag was expressed transiently using FreeStyle293-F cells (ThermoFisher). Conditioned media expressing recombinant human C1s M1 to V173+N174Q-Flag was applied to a column packed with anti-Flag M2 affinity resin (Sigma) and eluted with Flag peptide (Sigma) containing 1× PBS buffer. Fractions containing recombinant human C1s M1 to V173+N174Q-Flag were collected, stored at 4 degrees C., and used for analysis of antibody binding in reducing western blot.

### Example 1.7

### Expression and Purification of Recombinant Human C1r CCP1-CCP2-SP-Flag

The sequence used for expression and purification is human C1r CCP1-CCP2-SP I307-D705 (NCBI Reference Sequence: NP_001724.3) which has an N-terminus CAMPATH-1H signal sequence: MGWSCIILFLVATATGVHS (SEQ ID NO: 11) and C-terminus Flag tag linked with GGGGS linker. The human C1r sequence has R463Q S654A mutations (Kardos et. al. J Immunol. 2001 Nov. 1; 167(9):5202-8) (SEQ ID NO:167). The recombinant human C1r CCP1-CCP2-SP with Flag-tag on C-terminus was expressed transiently using FreeStyle293-F cell line (Thermo Fisher). Conditioned media expressing recombinant human C1r CCP1-CCP2-SP was applied to anti-Flag M2 affinity resin (Sigma) and eluted with Flag peptide (Sigma). Fractions containing recombinant human C1r CCP1-CCP2-SP-Flag protein were collected and subsequently subjected to a Superdex 200 gel filtration column (GE healthcare) equilibrated with 1×TBS. Fractions containing human C1r CCP1-CCP2-SP-Flag protein were then pooled, concentrated, and stored at −80 degrees C.

### Example 2

### Generation of Anti-C1s and Anti-C1r2s2 Antibodies

### Example 2.1

### Generation of Anti-C1s Antibodies

Anti-C1s antibodies were selected and assayed as follows:

Six NZW rabbits were immunized intradermally with native human C1s Proenzyme (CompTech, A103). Four or five repeated doses were given over a 2-month period followed by blood and spleen collection. For B cell selection, recombinant human C1r2s2 Flag/His tetramer, biotinylated native human C1s proenzyme, biotinylated recombinant human C1s-His, biotinylated recombinant human C1s CCP1-CCP2-SP-His and recombinant cyno C1s-Flag were prepared and used. B cells which can bind to native human C1s proenzyme, recombinant human C1r2s2 Flag/His tetramer, recombinant human C1s-His or recombinant cyno C1s-Flag were stained and sorted using a cell sorter and then plated and cultured according to the procedure described in WO2016098356A1. After cultivation, the B cell culture supernatants were collected for further analysis and the B cell pellets were cryopreserved.

Recombinant human C1r2s2 Flag/His tetramer and recombinant cyno C1s-Flag binding were evaluated by ELISA using the B cell culture supernatants. B cells which can bind to recombinant human C1r2s2 Flag/His tetramer and recombinant cyno C1s-Flag were selected for epitope analysis.

Epitope characterization was conducted by ELISA. Recombinant human C1s CCP1-CCP2-SP-His which is described above were used for this characterization. B cell lines were categorized into C1s CUB1-EGF-CUB2 binders or C1s CCP1-CCP2-SP binders.

The neutralizing activity for C1s CUB1-EGF-CUB2 binders was checked by neutralizing assay using selected B cell supernatants. Procedure of neutralizing assay was followed to RBC lysis assay described below. B cells with good neutralizing activities were preferred and selected for gene cloning.

### Example 2.2

### Generation of Anti-C1r2s2 Antibodies

Anti-C1r2s2 antibodies were selected and assayed as follows:

Three NZW rabbits were immunized intradermally with recombinant human C1r2s2 Flag/His tetramer described above. Five repeated doses were given over a 2-month period followed by blood and spleen collection. B cells which can bind to recombinant human C1r2s2 Flag/His tetramer and not bind to recombinant human C1s CCP1-CCP2-SP-His, or B cells which can bind to human C1r2s2 Flag/His tetramer were stained and sorted using a cell sorter and then plated and cultured according to the procedure described in WO2016098356A1. After cultivation, the B cell culture supernatants were collected for further analysis and the B cell pellets were cryopreserved.

Recombinant human C1r2s2 Flag/His tetramer and recombinant cyno C1r2s2 His/Flag tetratmer binding were evaluated by ELISA using the B cell culture supernatants. B cells which have cross reactivity were selected for epitope analysis.

ELISA based epitope characterization was conducted. Recombinant human C1s CCP1-CCP2-SP-His, recombinant human C1s-Flag and recombinant cyno C1s-Flag which are described above were prepared and used for this characterization. B cell lines which are C1s CUB1-EGF-CUB2 binders and B cell lines which are C1r binders were identified. Furthermore C1r binders are classified to C1r CUB1-EGF-CUB2 binders and C1r CCP1-CCP2-SP binders based on binding ability to recombinant human C1r CCP1-CCP2-SP-FLAG.

The neutralizing activity for C1s CUB1-EGF-CUB2 binders and C1r CUB1-EGF-CUB2 binders was checked by neutralizing assay using selected B cell supernatants. Procedure of neutralizing assay was followed to RBC lysis assay described below. B cells with good neutralizing activities were preferred and selected for gene cloning.

### Example 2.3.1

### Gene Cloning and Sequencing of C1s CUB1-EGF-CUB2 Binders

The RNAs of selected B cell lines with desired binding specificities and functions were purified from the cryopreserved cell pellets using the ZR-96 Quick-RNA kits (ZYMO RESEARCH, Cat No. R1053). These were named COS0221-0681. DNAs encoding antibody heavy-chain variable regions in the selected lines were amplified by reverse transcription PCR and recombined with a DNA encoding IgG4 (SEQ ID NO: 14), SG136 (SEQ ID NO: 15), and/or SG1148 (SEQ ID NO: 16) heavy-chain constant region. SG136 Fc contains mutations to reduce both C1q and Fc gamma receptor binding. SG1148 Fc contains mutation to reduce C1q binding while retaining Fc gamma receptor binding. DNAs encoding antibody light-chain variable regions were also amplified by reverse transcription PCR and recombined with a DNA encoding the k0MC light-chain constant region (SEQ ID NO: 17). Through further evaluation described below, five clones (COS0448, COS0499, COS0547, COS0631 and COS0637) were selected based on their binding ability, specificity and functionality. One clone (COS0583: VH, SEQ ID NO: 22; VL, SEQ ID NO: 29) was used as an assay control. The sequence ID numbers of VH, VL, HVR-H1, HVR-H2, HVR-H3, HVR-L1, HVR-L2, and HVR-L3 of the five antibodies are listed in Table 2.

TABLE 2

| Antibody | SEQ ID NO: | | | | | | | |
|---|---|---|---|---|---|---|---|---|
| name | VH | VL | HVR-H1 | HVR-H2 | HVR-H3 | HVR-L1 | HVR-L2 | HVR-L3 |
| COS0448 | 19 | 26 | 32 | 33 | 34 | 35 | 36 | 37 |
| COS0499 | 20 | 27 | 38 | 39 | 40 | 41 | 42 | 43 |
| COS0547 | 21 | 28 | 44 | 45 | 46 | 47 | 48 | 49 |
| COS0631 | 23 | 30 | 50 | 51 | 52 | 53 | 54 | 55 |
| COS0637 | 24 | 31 | 56 | 57 | 58 | 59 | 60 | 61 |

### Example 2.3.2

### Gene Cloning and Sequencing of C1r CUB1-EGF-CUB2 Binders

The RNAs of selected B cell lines with desired binding specificities and functions were purified from the cryopreserved cell pellets using the ZR-96 Quick-RNA kits (ZYMO RESEARCH, Cat No. R1053). These were named COR0001-0094, 0189-0376. DNAs encoding antibody heavy-chain variable regions in the selected lines were amplified by reverse transcription PCR and recombined with a DNA encoding IgG4 (SEQ ID NO: 14), SG136 (SEQ ID NO: 15), and/or SG1148 (SEQ ID NO: 16) heavy-chain constant region. SG136 Fc contains mutations to reduce both C1q and Fc gamma receptor binding. SG1148 Fc contains mutation to reduce C1q binding while retaining Fc gamma receptor binding. DNAs encoding antibody light-chain variable regions were also amplified by reverse transcription PCR and recombined with a DNA encoding the k0MC light-chain constant region (SEQ ID NO: 17). Through further evaluation described below, eight clones (COR0011, COR0058, COR0067, COR0205, COR0208, COR0212, COR0278 and COR0338) were selected based on their binding ability, specificity and functionality. The sequence ID numbers of VH, VL, HVR-H1, HVR-H2, HVR-H3, HVR-L1, HVR-L2, and HVR-L3 of the eight antibodies are listed in Table 3.

TABLE 3

| Antibody | SEQ ID NO: | | | | | | | |
|---|---|---|---|---|---|---|---|---|
| name | VH | VL | HVR-H1 | HVR-H2 | HVR-H3 | HVR-L1 | HVR-L2 | HVR-L3 |
| COR0011 | 103 | 111 | 119 | 127 | 135 | 143 | 151 | 159 |
| COR0058 | 104 | 112 | 120 | 128 | 136 | 144 | 152 | 160 |
| COR0067 | 105 | 113 | 121 | 129 | 137 | 145 | 153 | 161 |
| COR0205 | 106 | 114 | 122 | 130 | 138 | 146 | 154 | 162 |
| COR0208 | 107 | 115 | 123 | 131 | 139 | 147 | 155 | 163 |
| COR0212 | 108 | 116 | 124 | 132 | 140 | 148 | 156 | 164 |
| COR0278 | 109 | 117 | 125 | 133 | 141 | 149 | 157 | 165 |
| COR0338 | 110 | 118 | 126 | 134 | 142 | 150 | 158 | 166 |

### Example 2.4

### Monoclonal Antibody Expression and Purification

Recombinant antibodies were expressed transiently using the Expi 293-F cells and Expifectamine 293 (Life technologies), according to the manufacturer's instructions. Culture supernatant or recombinant antibodies were used for screening. Recombinant antibodies were purified with protein A (GE Healthcare) and eluted in PBS, TBS or His buffer (20 mM Histidine, 150 mM NaCl, pH 6.0). Size exclusion chromatography was further conducted to remove high molecular weight and/or low molecular weight component, if necessary.

### Example 3

### Binding Specificity of Anti-C1s and Anti-C1r Antibodies

### Example 3.1

### Binding Specificity of Anti-C1s Antibodies (BIACORE®)

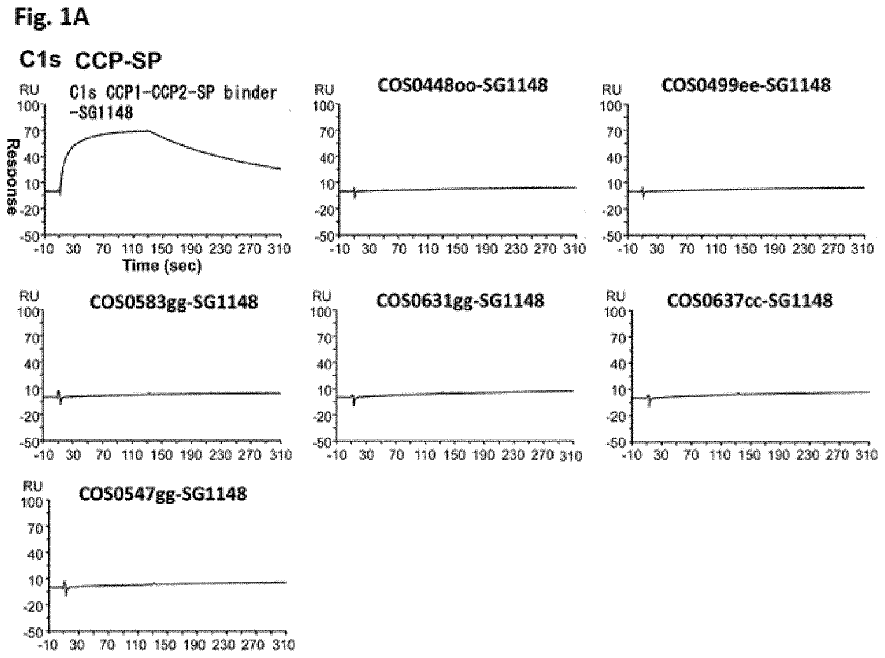

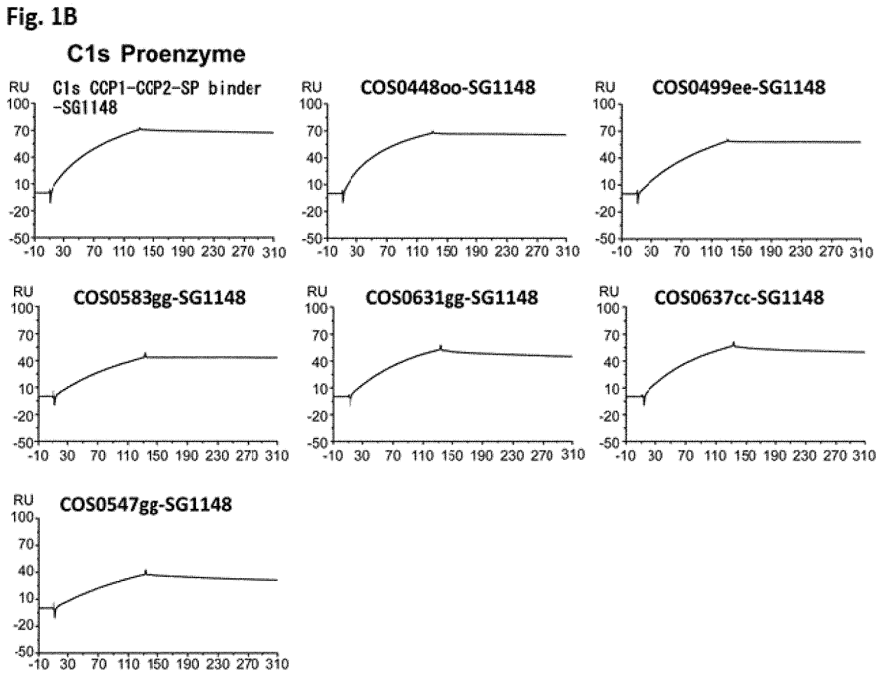

The binding specificities of the six C1s CUB1-EGF-CUB2 binders were determined at 37 degrees C. using BIACORE® T200 instrument (GE Healthcare). Recombinant Protein A/G (Pierce) was immobilized onto all flow cells of a CM4 sensor chip using an amine coupling kit (GE Healthcare). Antibodies and analytes were prepared in 7(+) buffer (20 mM ACES, 150 mM NaCl, 1.2 mM CaCl₂, 0.05% Tween 20, 0.005% NaN₃, pH 7.4). Each antibody was captured onto the sensor surface by protein A/G. Antibody capture levels were aimed at 100 resonance unit (RU). Native proenzyme human C1s (Comptech A103) (at 50 nM as a monomer) or recombinant human C1s CCP1-CCP2-SP-His (at 100 nM as a monomer) was injected, followed by dissociation. Sensor surface was regenerated each cycle with 10 mM Glycine-HCl pH 1.5. The results are shown in FIGS. 1A and 1B. The six C1s CUB1-EGF-CUB2 binders bound to the native proenzyme human C1s, but not recombinant human C1s CCP1-CCP2-SP-His, which is a truncated protein lacking the CUB1-EGF-CUB2 domain.

### Example 3.2

### Binding Specificity of Anti-C1r Antibodies (BIACORE®)

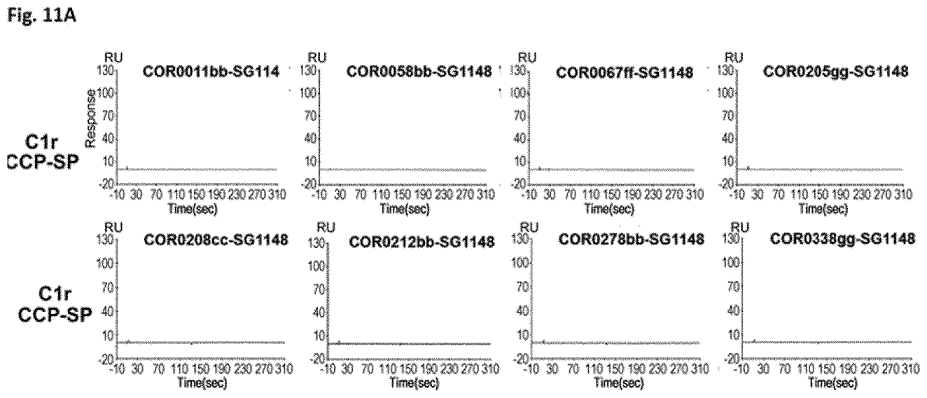

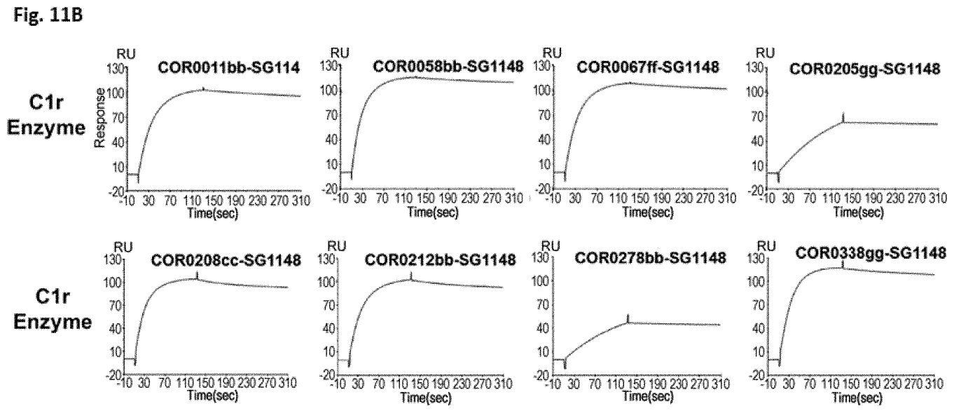

The binding specificities of human C1r CUB1-EGF-CUB2 binders are determined at 37 degrees C. using BIACORE® T200 instrument (GE Healthcare). Recombinant Protein A/G (Pierce) is immobilized onto all flow cells of a CM4 sensor chip using an amine coupling kit (GE Healthcare). Antibodies and analytes are prepared in 7(+) buffer (20 mM ACES, 150 mM NaCl, 1.2 mM CaCl₂, 0.05% Tween 20, 1 mg/mL BSA (IgG-free), 1 mg/mL CMD, 0.005% NaN₃, pH 7.4). Each antibody is captured onto the sensor surface by protein A/G. Antibody capture levels are aimed at 100 resonance unit (RU). Native human C1r enzyme (Comptech A102) (at 25 nM as a dimer) or recombinant human C1r CCP1-CCP2-SP-FLAG (at 50 nM as a monomer) is injected, followed by dissociation. Sensor surface is regenerated each cycle with 10 mM Glycine-HCl pH 1.5. The results are shown in FIGS. 11A and 11B. It may be determined that C1r CUB1-EGF-CUB2 binders bound to the native human C1r enzyme , but not recombinant human C1r CCP1-CCP2-SP-FLAG, which is a truncated protein lacking the CUB1-EGF-CUB2 domain of C1r.

### Example 4

### Evaluation of C1q Displacement Function of Anti-C1s and Anti-C1r Antibodies (BIACORE®—C1r2s2 Immobilized)

### Example 4.1

### Evaluation of C1q Displacement Function of Anti-C1s Antibodies (BIACORE®—C1r2s2 Immobilized)

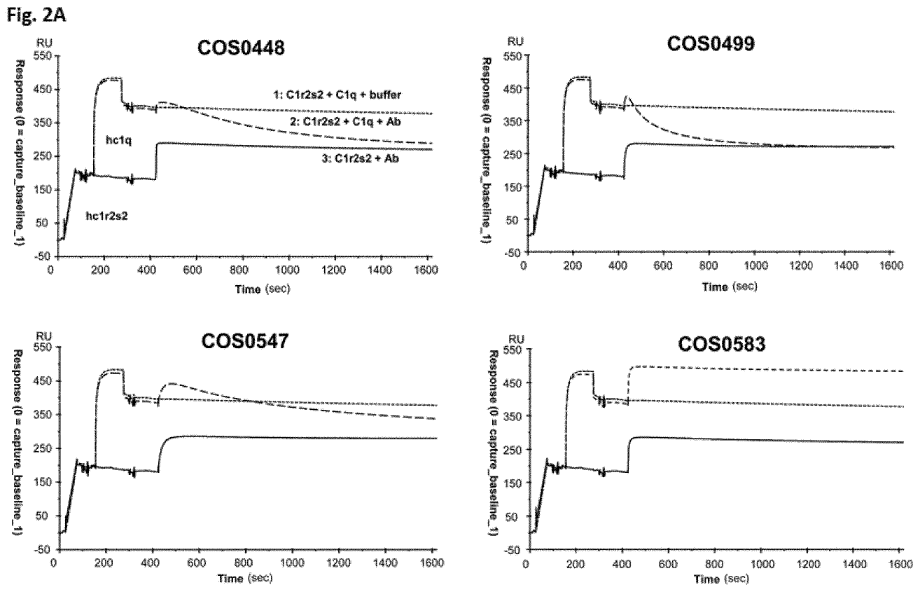

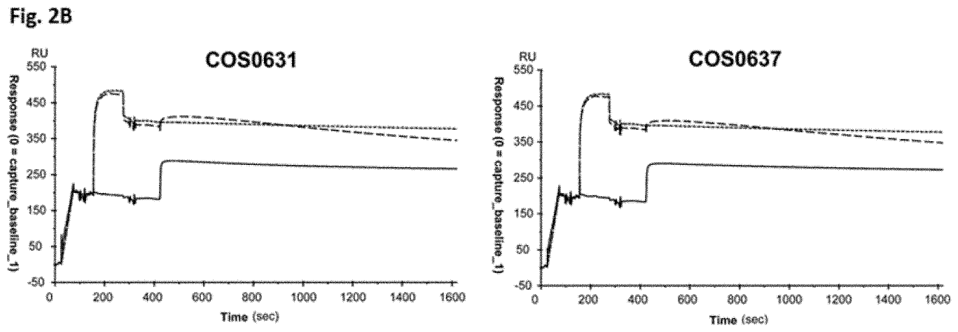

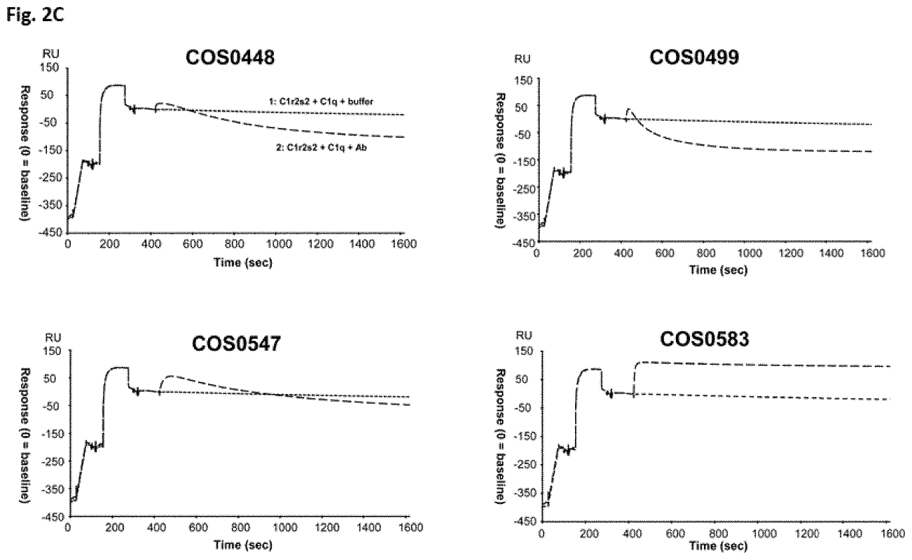

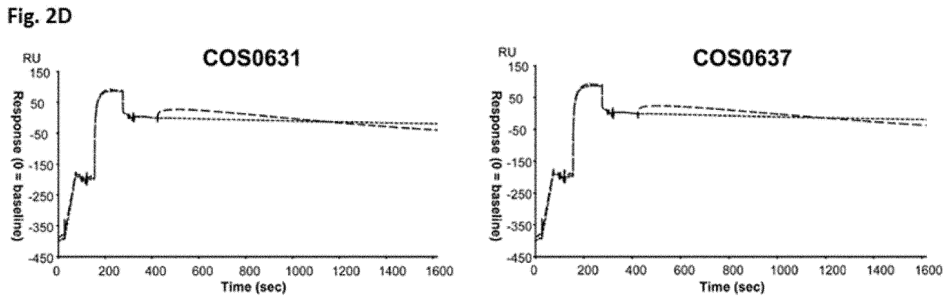

The C1q displacement function of the antibodies was demonstrated by a C1r2s2 capture method using BIACORE® T200 instrument (GE Healthcare) at 37 degrees C. An anti-His antibody (GE-Healthcare) was immobilized onto all flow cells of a CM4 sensor chip using an amine coupling kit (GE Healthcare). The antibodies, recombinant human C1r2s2 Flag/His tetramer and native human C1q (Comptech A099) were prepared in pH 7.4 buffer (20 mM ACES, 150 mM NaCl, 1.2 mM CaCl₂, 1 mg/mL BSA (IgG-free), 1 mg/mL CMD, 0.05% Tween 20, 0.005% NaN₃, pH 7.4). Recombinant human C1r2s2 Flag/His tetramer was first captured onto the sensor surface by the anti-His antibody (“hc1r2s2” in FIG. 2A). The capture levels were aimed at 200 resonance unit (RU). Native human C1q was injected at 100 nM to have a capture of 200 RU (“hc1q” in FIG. 2A), followed by antibody injection at 500 nM at 10 micro L/min for 1200 sec immediately. The sensor surface was regenerated each cycle with 10 mM Glycine-HCl (pH 1.5). The results are shown in FIGS. 2A to 2D. For the antibodies with the C1q displacement function, the response unit of Sensorgram 2 (large dotted line; “C1r2s2+C1q+Ab” in FIGS. 2A and 2C) is lower than the response unit in Sensorgram 1 (small dotted line; “C1r2s2+C1q+buffer” in FIGS. 2A and 2C) after the time point where Sensorgrams 1 and 2 cross (“time point of crossover”). Depending on the time when Sensorgram 2 crosses over Sensorgram 1, COS0499 was categorized as a fast displacement variant. COS0547, COS0631 and COS0637 showed comparatively slow displacement. COS0448 was in the middle of fast displacement and slow displacement.

The time point of crossover is identified by subtraction of the buffer response (Sensorgram 1) from the antibody (Ab) response (Sensorgram 2), and referring to the time point when the differential value changes from positive to negative (Table 4). The time from the start of Ab injection is indicated in Table 4 as “time point of crossover”.

Note that, herein, COS448, COS499, COS0547, COS583, COS0631, and COS0637 may alternatively be called COS44800(−SG1148), COS499ee(−SG1148), COS0547gg(−SG1148), COS583gg(−SG1148), COS0631gg(−SG1148), and COS0637cc(−SG1148), respectively.

TABLE 4
Time point of crossover for 6 C1s CUB1-EGF-CUB2 binders

| | Time point of crossover |
|---|---|
| Antibody name | (post-injection) (sec) |
| COS0448oo-SG1148 | 168.9 |
| COS0499ee-SG1148 | 61.9 |
| COS0547gg-SG1148 | 542 |
| COS0583gg-SG1148 | ND |
| COS0631gg-SG1148 | 736.4 |
| COS0637cc-SG1148 | 764 |

### Example 4.2

### Evaluation of C1q Displacement Function of Anti-C1r Antibodies (BIACORE®—C1r2s2 Immobilized)

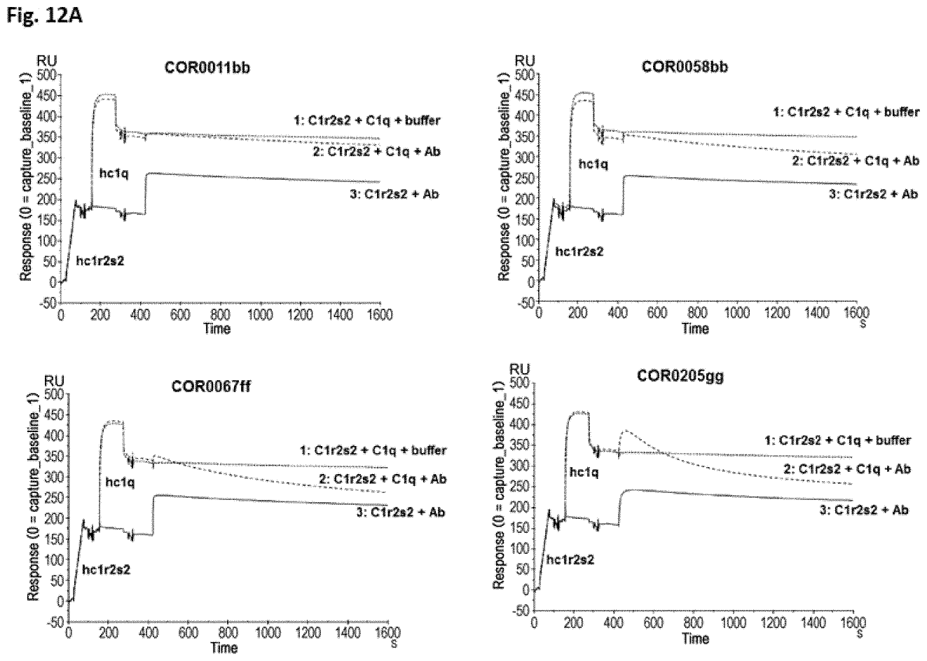

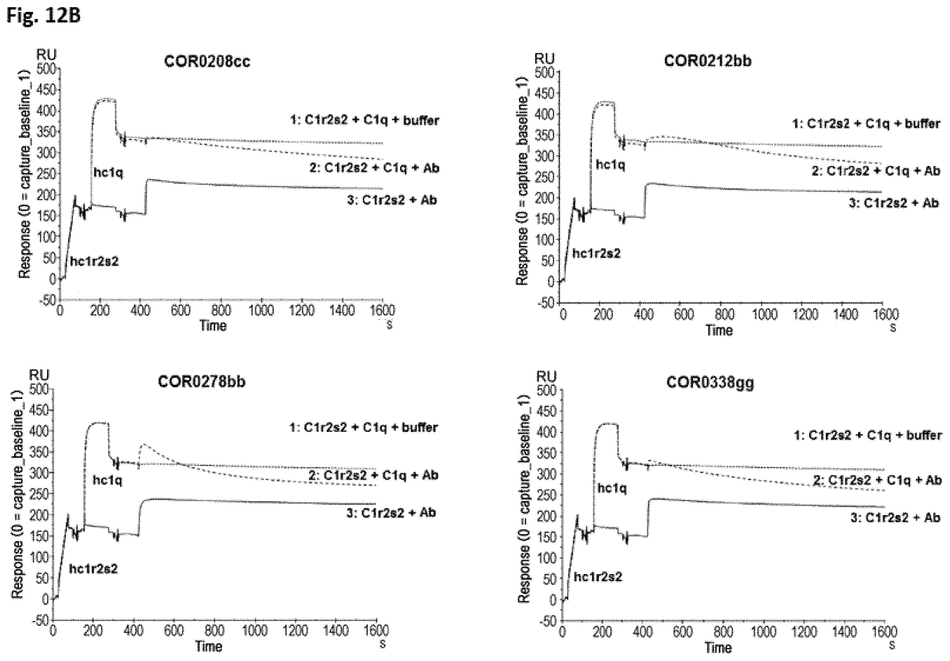

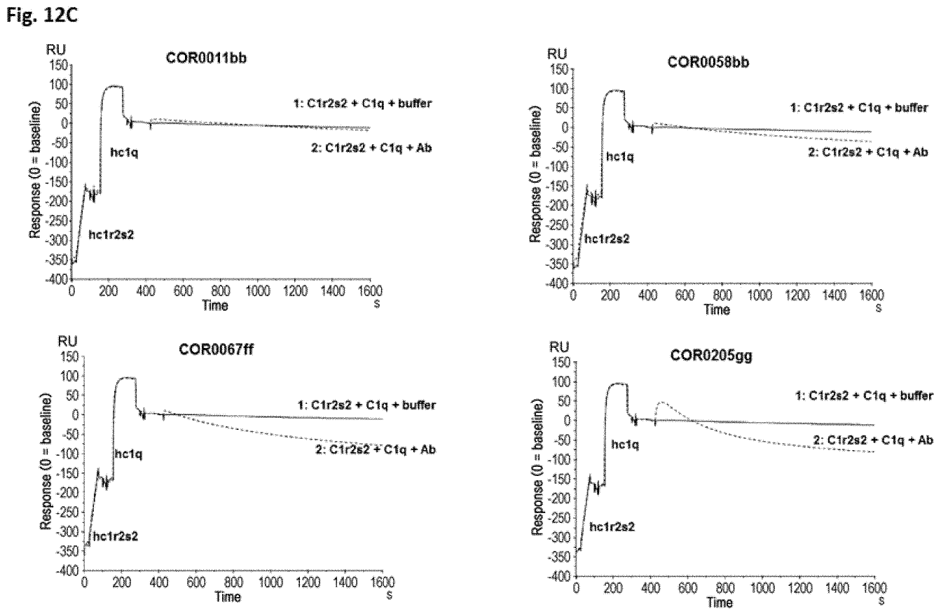

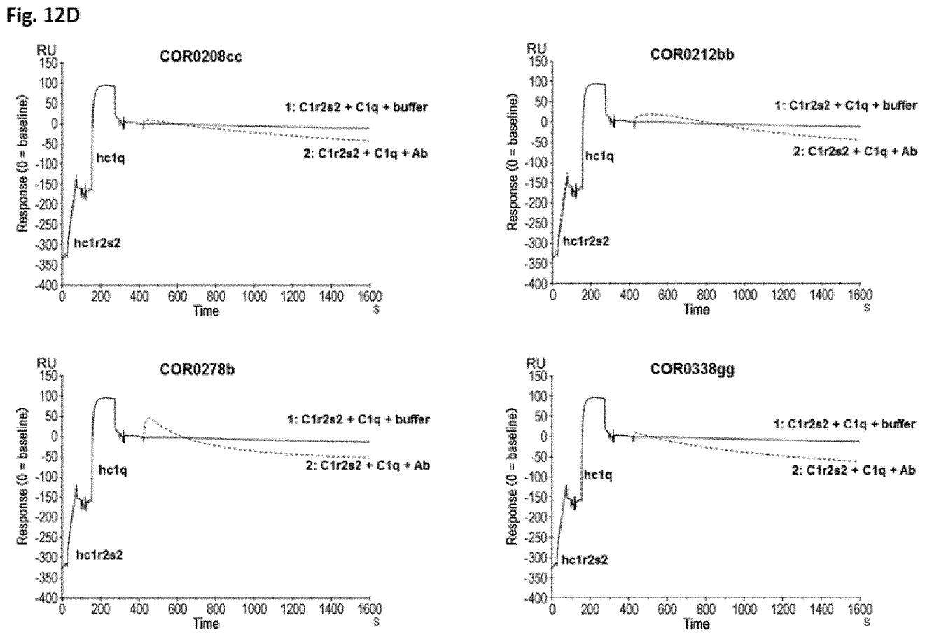

The C1q displacement function of antibodies is demonstrated by a C1r2s2 capture method using BIACORE® T200 instrument (GE Healthcare) at 37 degrees C. An anti-His antibody (GE-Healthcare) is immobilized onto all flow cells of a CM4 sensor chip using an amine coupling kit (GE Healthcare). The antibodies, recombinant human C1r2s2 Flag/His tetramer and native human C1q (Comptech A099) are prepared in pH 7.4 buffer (20 mM ACES, 150 mM NaCl, 1.2 mM CaCl₂, 1 mg/mL BSA (IgG-free), 1 mg/mL CMD, 0.05% Tween 20, 0.005% NaN₃, pH 7.4). Recombinant human C1r2s2 Flag/His tetramer is first captured onto the sensor surface by the anti-His antibody (“hc1r2s2”). The capture levels are aimed at 200 resonance unit (RU). Native human C1q is injected at 100 nM to have a capture of 200 RU (“hc1q”), followed by antibody injection at 500 nM at 10 micro L/min for 1200 sec immediately. The sensor surface is regenerated each cycle with 10 mM Glycine-HCl (pH 1.5). The results are shown in FIGS. 12A to 12D. For antibodies with the C1q displacement function, the response unit of Sensorgram 2 (in the presence of C1r2s2, C1q, and antibody) is lower than the response unit in Sensorgram 1 (in the presence of C1r2s2, C1q, and buffer, but in the absence of antibody) after the time point where Sensorgrams 1 and 2 cross (“time point of crossover”).

The time point of crossover is identified by subtraction of the buffer response (Sensorgram 1) from the antibody (Ab) response (Sensorgram 2), and referring to the time point when the differential value changes from positive to negative (Table 5).

Note that, herein, COR0011, COR0058, COR0067, COR0205, COR208, COR0212, COR0278, and COR0338 may alternatively be called COR0011bb(−SG1148), COR0058bb(−SG1148), COR0067ff(−SG1148), COR205gg(−SG1148), COR208cc(−SG1148), COR0212bb(−SG1148), COR0278bb(−SG1148), and COR0338gg(−SG1148), respectively.

TABLE 5
Time point of crossover for 8 C1r CUB1-EGF-CUB2 binders

| | Time point of crossover |
|---|---|
| Antibody name | (post-injection) (sec) |
| COR0011bb-SG1148 | 612.7 |
| COR0058bb-SG1148 | 234.8 |
| COR0067ff-SG1148 | 85.0 |
| COR0205gg-SG1148 | 215.9 |
| COR0208cc-SG1148 | 196.9 |
| COR0212bb-SG1148 | 432.1 |
| COR0278bb-SG1148 | 217.6 |
| COR0338gg-SG1148 | 100.7 |

### Example 5

### Evaluation of C1q Displacement Function of Anti-C1s Antibodies (BIACORE®—C1q Immobilized)

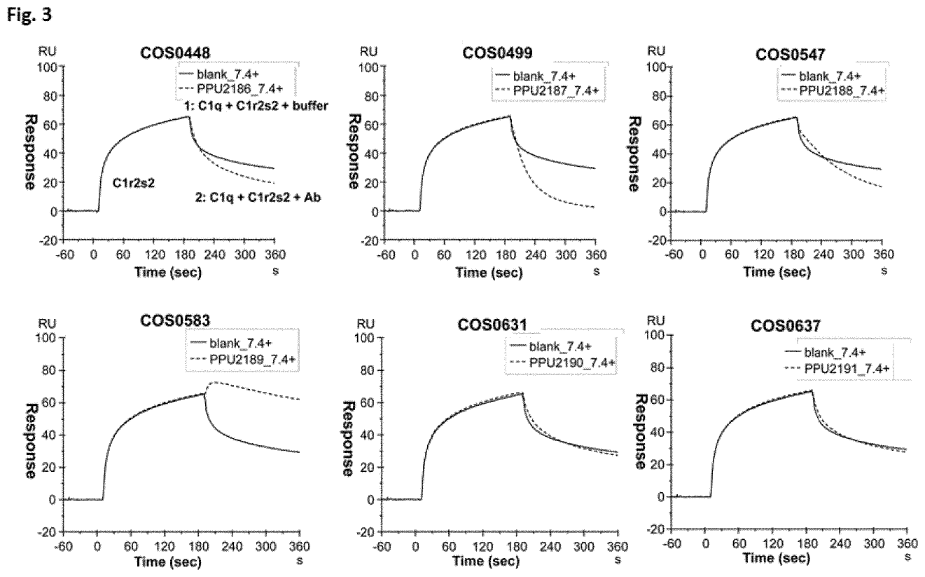

The C1q displacement function of the antibodies was demonstrated by a C1q capture method using BIACORE® T200 instrument (GE Healthcare) at 37 degrees C. The antibodies, recombinant human C1r2s2 Flag/His tetramer and native human C1q (Comptech A099) that has been biotinylated were prepared in pH 7.4 buffer (20 mM ACES, 150 mM NaCl, 1.2 mM CaCl₂, 1 mg/mL BSA (IgG-free), 1 mg/mL CMD, 0.05% Tween 20, 0.005% NaN₃, pH 7.4). Biotinylated native human C1q was first captured onto one flow cell of a CAP sensor chip (GE-Healthcare). The capture levels were aimed in the range of 800 to 1000 resonance unit (RU). Recombinant human C1r2s2 Flag/His tetramer was injected at 300 nM, followed by antibody injection at 500 nM at 10 micro L/min for 180 sec. The sensor surface was regenerated each cycle with 8 M Guanidine-HCl and 1 M NaOH in 3-to-1 ratio. The sensograms after blank subtraction by using BIACORE® T200 Evaluation software, version 2.0 (GE Healthcare) are shown in FIG. 3. The antibodies with the C1q displacement function enhanced the dissociation rate of C1r2s2, i.e., the “2: C1q+C1r2s2+Ab” (dotted line) curve runs below the “1: C1q+C1r2s2+buffer” (solid line) curve in FIG. 3. COS0499 is a fast displacement variant. COS0631 and COS0637 are slow displacement variants. COS0448 and COS0547 showed medium rate displacement.

### Example 6

### Evaluation of C1q Blocking Function of Anti-C1s Antibodies (BIACORE®)

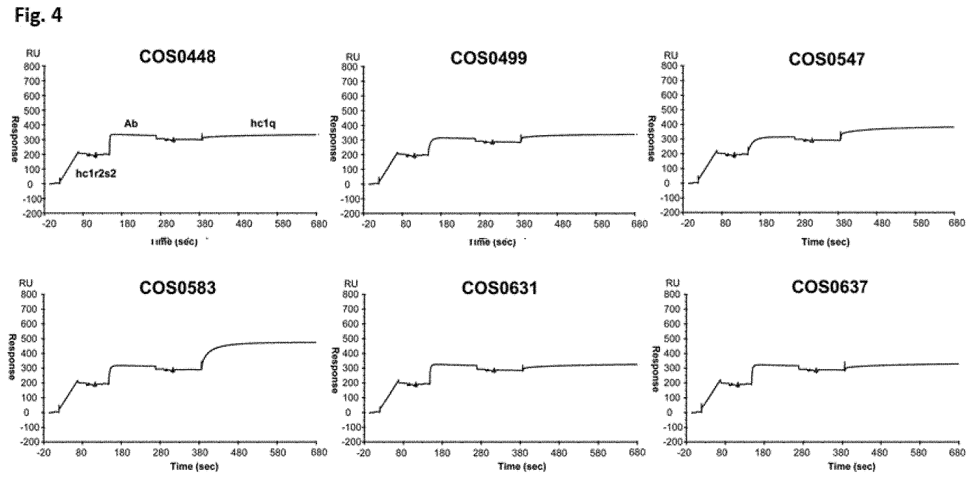

To assess the blocking of C1q binding to C1r2s2 by the antibodies, blocking assay was performed at 37 degrees C. using BIACORE® T200 instrument (GE Healthcare). An anti-His antibody (GE-Healthcare) was immobilized onto all flow cells of a CM4 sensor chip using an amine coupling kit (GE Healthcare). The antibodies, recombinant human C1r2s2 Flag/His tetramer and native human C1q were prepared in pH 7.4 buffer (20 mM ACES, 150 mM NaCl, 1.2 mM CaCl2, 1 mg/mL BSA (IgG-free), 1 mg/mL CMD, 0.05% Tween 20, 0.005% NaN₃, pH 7.4). Recombinant human C1r2s2 Flag/His tetramer was first captured onto the sensor surface by the anti-His antibody (“hc1r2s2” in FIG. 4). The capture levels were aimed at 200 resonance unit (RU). The antibody variants were injected at 500 nM (“Ab” in FIG. 4), followed by native human C1q injection at 100 nM (“hc1q” in FIG. 4). The sensor surface was regenerated each cycle with 10 mM Glycine-HCl (pH 1.5). Antibodies with C1q blocking function are those which compete with C1q for binding to C1r2s2. The results are shown in FIG. 4. COS0448, CPS0631, COS0637 and COS0499 showed strong blocking function against C1q binding. Partial blocking was observed in COS0547. On the other hand, COS0583 did not show the blocking function.

### Example 7

### Evaluation of Complement Neutralization Function (RBC Lysis Inhibition)

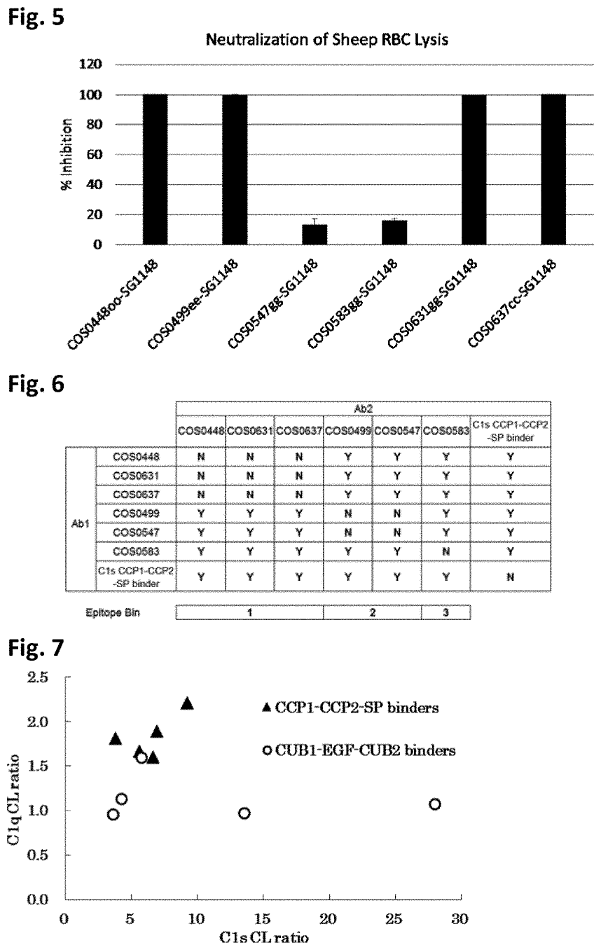

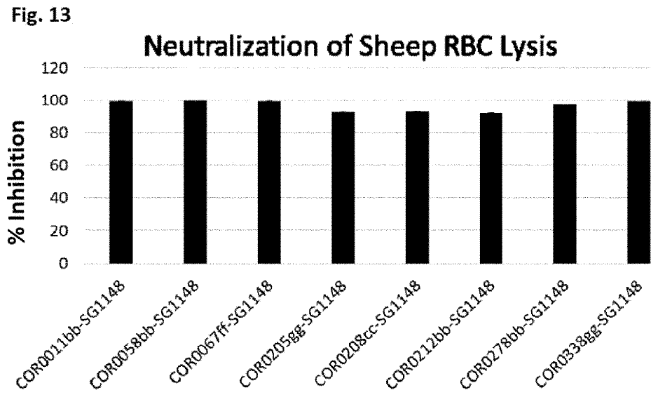

The neutralization function of the antibodies was assessed as follows. Either sensitized sheep or chicken RBCs were used to evaluate the complement inhibitory activity of antibodies. The following method describes the protocol used for sheep RBC lysis assay. Human serum (Biopredic) was diluted to 8% with assay buffer (HBSS Ca2+Mg2+ with 0.05% BSA) and pre-incubated with equal volume of antibodies diluted to 40 micro g/mL for 3 hours at 37 degrees C. As a control, human serum was pre-incubated with assay buffer alone, or assay buffer with 10 mM EDTA. Sheep red blood cells (Innovative Research) were sensitized with anti-sheep red blood cell stroma antibody (Abcam), rinsed, counted and adjusted to 5×108 cells/mL in assay buffer. The antibody/serum mixture was then added to an equal volume of sensitized sheep red blood cells (Innovative Research) and incubated for one hour at 37 degrees C. to allow for lysis of the red blood cells. The final concentration of human serum and antibodies in this reaction was 2% and 10 micro g/mL respectively. The reaction was stopped with cold assay buffer containing EDTA. The mixture was centrifuged to pellet unlysed cells and the supernatant was withdrawn, and absorbance (OD) at 415 nm, from which OD at 630 nm was subtracted, was used to analyze the release of hemoglobin. To calculate the percentage inhibition of red blood cell lysis, 0% inhibition was set as the condition where no antibody (buffer only) was added, and 100% inhibition was set as the condition where EDTA was added at a final concentration of 5 mM. Data shown in FIG. 5 and FIG. 13 are represented as MEAN+SD from 2 replicate wells.

### Example 8

### Competitive Epitope Analysis

Competitive epitope binning experiment was performed by real-time binding assay using Octet (Pall ForteBio). Biotinylated recombinant human C1s-Flag was prepared and captured onto streptavidin biosensor tips (Pall ForteBio). The antigen captured tips were dipped into 25 microgram (micro g)/mL of first set of antibodies for 200 seconds. Then the tips were incubated with 25 micro g/mL of second set of antibodies for 200 seconds. To eliminate an effect of antibody dissociation, second set of antibodies include same concentration of first antibodies. Result was analysed by DATA Analysis HT software (Pall ForteBio, Version 10.0.1.7). Positive response of second set of antibodies indicate different epitopes and negative response of second set of antibodies shows the same epitopes. Antibodies that bind to the CUB1-EGF-CUB2 domain of C1s were classified into 3 “epitope bins”. Displacement antibodies are located within epitope bins 1 and 2. In FIG. 6, “Ab1” indicates first set of antibodies and “Ab2” indicates second set of antibodies. the letters ‘Y’ and ‘N’ indicate yes/no for the binding of the respective antibody pairs in tandem (i.e., “no” means that the antibodies compete with each other and do not bind to the antigen “in tandem”, and thus they are located in the same epitope bin). C1s CCP1-CCP2-SP binder (VH: SEQ ID NO: 18, VL: SEQ ID NO: 25), which binds within the CCP1-CCP2-SP domain but not the CUB1-EGF-CUB2 domain of C1s, was added as a control antibody.

### Example 9

### Mice PK Study Using Anti-C1s CUB1-EGF-CUB2 and CCP1-CCP2-SP Binders Antibody

### Measurement of Total Human C1s and C1q Concentration in Mouse Plasma by High-Performance Liquid Chromatography-Electrospray Tandem Mass Spectrometry (LC/ESI-MS/MS)

The total concentrations of human C1s and C1q in mouse plasma was measured by LC/ESI-MS/MS. The calibration standards were prepared by mixing and diluting human C1s and C1q in defined amounts in mouse plasma, resulting in human C1s concentrations of 0.477, 0.954, 1.91, 3.82, 7.64, 15.3, 30.5 micrograms (micro g)/mL and human C1q concentrations of 0.977, 1.95, 3.91, 7.81, 15.6, 31.3 and 62.5 micro g/mL, respectively. A 2 micro L of the calibration standards and plasma samples was mixed with 25 micro L of 6.8 mol/L Urea, 9.1 mmol/L dithiothreitol and 0.4 micro g/mL lysozyme (chicken egg white) in 50 mmol/L ammonium bicarbonate and incubated for 45 min at 56 degrees C. Then, 2 micro L of 500 mmol/L iodoacetamide was added and incubated for 30 min at 37 degrees C. in the dark. Next, 160 micro L of 0.5 micro g/mL sequencing grade modified trypsin (Promega) in 50 mmol/L ammonium bicarbonate was added and incubated at 37 degrees C. overnight. Finally, 5 micro L of 10% trifluoroacetic acid was added to deactivate any residual trypsin. A 40 micro L of digestion samples were subjected to analysis by LC/ESI-MS/MS. LC/ESI-MS/MS was performed using Xevo TQ-S triple quadrupole instrument (Waters) equipped with 2D I-class UPLC (Waters). Human C1s specific peptide LLEVPEGR and human C1q specific peptide IAFSATR were monitored by the selected reaction monitoring (SRM). SRM transition was [M+2H]2+ (m/z 456.8) to y6 ion (m/z 686.3) for human C1s, and [M+2H]2+ (m/z 383.2) to y5 ion (m/z 581.3) for human C1q. Calibration curve was constructed by the weighted (1/×2) linear regression using the peak area plotted against the concentrations. The concentration in mouse plasma was calculated from the calibration curve using the analytical software Masslynx Ver.4.1 (Waters).

Evaluation of Pharmacokinetics for Total hC1s and hC1q After Administration of Anti-C1s Antibodies in Mice

The in vivo pharmacokinetics of hC1s, hC1q and anti-C1s antibodies prepared in Example 1 was assessed after administering antigen alone (hC1q, recombinant C1r2s2, mixture of hC1q and rC1r2s2) or with anti-C1s antibody to mice (CB17/Icr-Prkdcscid/CrlCrlj: Charles River Japan). Three mice were allocated to each dosing group.

Firstly, hC1q solution (0.84 mg/mL), rC1r2s2 (0.47 mg/mL) or a solution of mixture containing hC1q and rC1r2s2 (0.84 and 0.47 mg/mL, respectively) was injected at a dose of 10 mL/kg to mice intravenously. After dosing of antigen solution, anti-C1s antibody solution (2.5 mg/mL) was immediately administered to the same individual in the same way.

The dose setting of C1q and rC1r2s2 was designed to be physiological concentration in human plasma just after administration. Dosage of anti-C1s antibody was adjusted to be excess concentration over both antigens during the study, and thus almost all hC1s was assumed to be bound form in circulation.

Blood was collected at 5, 30 minutes, 2, 7 hours, 3, 7, 14, 21 and 28 days after injection. These blood was centrifuged immediately to separate the plasma samples. Plasma concentrations of hC1s and hC1q were measured at each sampling points by LC/ESI-MS/MS. PK parameters of hC1s and hC1q were estimated by non-compartmental analysis (Phoenix WinNonlin version 8.0, Certara).

The following antibodies were administered to mice as anti-C1s antibodies (Tables 2 and 7):

1. COS0098bb-SG1148/SG136

2. COS0112gg-SG1148/SG136

3. COS0127bb-SG1148/SG136

4. COS0158ee-SG1148/SG136

5. COS0182hh-SG1148/SG136

6. COS0448oo-SG1148/SG136

7. COS0499ee-SG1148/SG136

8. COS0547gg-SG1148/SG136

9. COS0631gg-SG1148/SG136

10. COS0637cc-SG1148/SG136

The mice PK study results are shown in FIG. 7. PK parameters of hC1q and hC1s are shown in Table 6. hC1s CL ratio (SG1148/SG136) of 5 CCP1-CCP2-SP binders (COS0098bb, COS0112gg, COS0127bb, COS0158ee and COS0182hh) was 9.2, 6.9, 5.6, 3.8 and 6.6, respectively.

hC1s CL ratio (SG1148/SG136) of 5 CUB1-EGF-CUB2 binders (COS0448oo, COS0499ee, COS0547gg, COS0631gg and COS0637cc) was 4.2, 5.8, 3.6, 13.6 and 28.0, respectively. This value indicate potential to accelerate hC1s elimination via Fc gamma receptor. COS0098bb and COS0637cc are considered to have highest potential among CCP1-CCP2-SP and CUB1-EGF-CUB2 binders, respectively. hC1q CL ratio (SG1148/SG136) was evaluated in the same way. All CCP1-CCP2-SP binders accelerated C1q elimination around 2-fold compared to those of SG136. On the other hand, CUB1-EGF-CUB2 binders did not affect C1q CL except for COS0499ee. From these study, CUB1-EGF-CUB2 binders might have less impact on C1q pharmacokinetics compared to CCP1-CCP2-SP binders.

TABLE 6

| | C1s CL ratio | C1q CL ratio |
|---|---|---|
| | (SG1148/SG136) | (SG1148/SG136) |
| COS0098bb | 9.217 | 2.209 |
| COS0112gg | 6.928 | 1.890 |
| COS0127bb | 5.586 | 1.663 |
| COS0158ee | 3.796 | 1.811 |
| COS0182hh | 6.629 | 1.594 |
| COS0448oo | 4.244 | 1.130 |
| COS0499ee | 5.773 | 1.593 |
| COS0547gg | 3.596 | 0.955 |
| COS0631gg | 13.552 | 0.970 |
| COS0637cc | 27.991 | 1.070 |

TABLE 7

| Antibody | SEQ ID NO: | | | | | | | |
|---|---|---|---|---|---|---|---|---|
| name | VH | VL | HVR-H1 | HVR-H2 | HVR-H3 | HVR-L1 | HVR-L2 | HVR-L3 |
| COS0098bb | 62 | 67 | 72 | 73 | 74 | 87 | 88 | 89 |
| COS0112gg | 63 | 68 | 75 | 76 | 77 | 90 | 91 | 92 |
| COS0127bb | 64 | 69 | 78 | 79 | 80 | 93 | 94 | 95 |
| COS0158ee | 65 | 70 | 81 | 82 | 83 | 96 | 97 | 98 |
| COS0182hh | 66 | 71 | 84 | 85 | 86 | 99 | 100 | 101 |

Name of constant region: SG1148 (CH: SEQ ID NO: 16 and CL SEQ ID NO: 102), SG136 (CH: SEQ ID NO: 15 and CL: SEQ ID NO: 102)

### Example 10

### Time Dependent Complement Neutralization Function (RBC Lysis Inhibition)

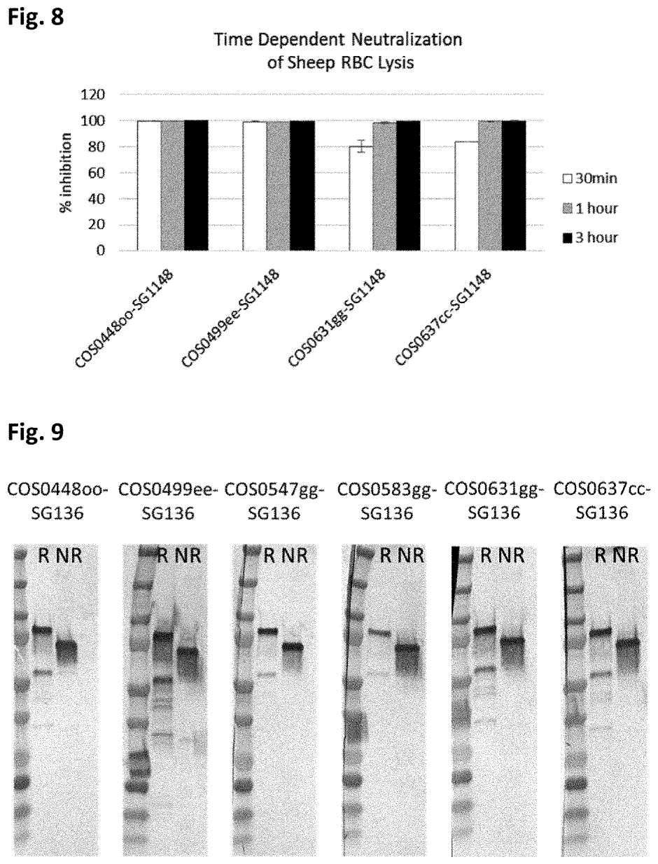

The time dependent neutralization function of the antibodies was assessed as follows. Human serum (Biopredic) was diluted to 8% with assay buffer (HBSS Ca²⁺Mg²⁺ with 0.05% BSA) and pre-incubated with equal volume of antibodies diluted to 40 micro g/mL for either 0.5, 1 or 3 hours at 37 degrees C. As a control, human serum was pre-incubated with assay buffer alone, or assay buffer with 10 mM EDTA. The antibody/serum mixture was then added to an equal volume of sensitized sheep red blood cells (Innovative Research) and incubated for one hour at 37 degrees C. to allow for lysis of the red blood cells. The final concentration of human serum and antibodies in this reaction was 2% and 10 micro g/mL respectively. The reaction was stopped with cold assay buffer containing EDTA. The mixture was centrifuged to pellet unlysed cells and the supernatant was withdrawn, and absorbance (OD) at 415 nm, from which OD at 630 nm was subtracted, was used to analyze the release of hemoglobin. Analysis of % inhibition and sheep red blood cells sensitization were performed as described before in EXAMPLE 7. Data shown are represented as MEAN+SD from 2 replicate wells. FIG. 8 illustrates the time dependent neutralization of human serum complement activity by the following anti-C1s antibodies: COS0448oo-SG1148; COS0499ee-SG1148; COS0631gg-SG1148; and COS0637cc-SG1148.

### Example 11

### Antibody Binding to Native Human Proenzyme C1s in Reducing and Non-Reducing Western Blotting Analysis

Western blot analysis of native human C1s proenzyme protein (CompTech) was performed under non-reducing (NR) and reducing conditions (R). C1s proenzyme was boiled in sample loading buffer containing SDS at 95 degrees C. either with or without 3-mercapto-1,2-propanediol (Wako). Each blot was incubated with the indicated anti-C1s antibodies at a concentration of 5 micro g/mL for 1 hour at room temperature, and detected by anti-human IgG alkaline phosphatase (Biorad) secondary antibody. FIG. 9 illustrates the antibody binding to native human proenzyme C1s in reducing and non-reducing western blotting analysis on the following antibodies: COS0448oo-SG136; COS0499ee-SG136; COS0547gg-SG136; COS0583gg-SG136; COS0631gg-SG136; and COS0637cc-SG136.

### Example 12

### Antibody Binding to Truncated C1s Proteins in Reducing Western Blot

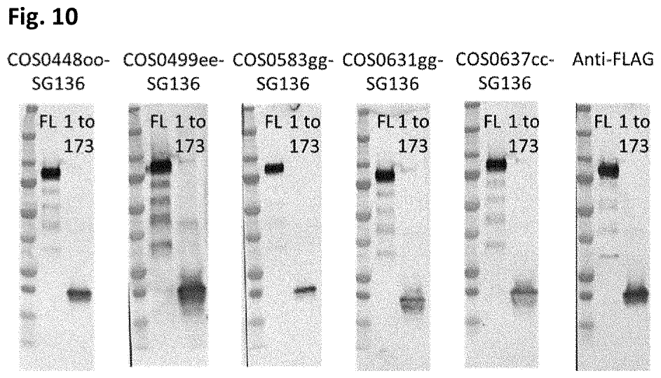

Binding of anti-C1s antibodies to truncated human C1s proteins were analyzed by reducing western blot analysis as follows. Recombinant full length human C1s-Flag and truncated human C1s M1 to V173+N174Q-Flag were boiled in sample loading buffer containing SDS and 3-mercapto-1,2-Propanediol (Wako). Each blot was incubated with the indicated anti-C1s antibody at a concentration of 1 micro g/mL for 1 hour at room temperature, and detected by F(ab′)2 Goat anti-human IgG Fc Alkaline Phosphatase (ThermoFisher) secondary antibody. As control, anti-Flag (M2) antibody (Sigma) Alkaline Phosphatase was used to detect the recombinant full length and truncated human C1s. The notation FL indicates full length C1s-Flag, and 1 to 173 indicates truncated human C1s M1 to V173+N174Q-Flag. FIG. 10 illustrates the antibody binding to truncated C1s proteins in reducing western blot on the following antibodies: COS0448oo-SG136; COS0499ee-SG136; COS0583gg-SG136; COS0631gg-SG136; and COS0637cc-SG136.

### [Sequence Listing]

1. An isolated antibody that inhibits the interaction between C1q and C1r2s2 complex, wherein the antibody has a displacement function such that the antibody binds to C1qrs complex and promotes dissociation of C1q from C1qrs complex.

2. The antibody of claim 1, wherein the antibody binds to C1qrs complex on a BIACORE® chip and promotes dissociation of C1q from C1qrs complex, wherein a value of response unit (RU) in presence of the antibody is lower than a value of response unit (RU) in the absence of the antibody as determined by a BIACORE® assay when a sufficient time passed.

3. The antibody of claim 2, wherein the time point of crossover in the BIACORE® assay is within 1000 s after the time point of the start of antibody injection as determined by the BIACORE® assay using the following conditions: the capture levels of C1r2s2 complex and C1q are at 200 resonance unit (RU) and 200 resonance unit (RU), respectively, and the antibody as an analyte is injected at 500 nM at 10 microliter/min.

4. The antibody of claim 2, wherein almost all of C1q are dissociated from C1qrs complex within 2000 s after the time point of the start of antibody injection as determined by the BIACORE® assay using the following conditions: the capture levels of C1r2s2 complex and C1q are at 200 resonance unit (RU) and 200 resonance unit (RU), respectively, and the antibody as an analyte is injected at 500 nM at 10 microliter/min.

5. An isolated antibody that inhibits the interaction between C1q and C1r2s2 complex, wherein the antibody has a neutralizing activity for human serum complement of at least 70% in an RBC assay.

6. The antibody of claim 1, wherein the antibody is an antibody that specifically binds to C1s or an antibody that specifically binds to C1r.

7. An isolated antibody that inhibits the interaction between C1q and C1r2s2 complex,
wherein the antibody specifically binds to an epitope within a CUB1-EGF-CUB2 domain of C1s, and competes for binding to the epitope with an antibody selected from the group consisting of 1)-5) below:
1) an antibody comprising the HVR-H1 sequence of SEQ ID NO: 32, the HVR-H2 sequence of SEQ ID NO: 33, the HVR-H3 sequence of SEQ ID NO: 34, the HVR-L1 sequence of SEQ ID NO: 35, the HVR-L2 sequence of SEQ ID NO: 36, and the HVR-L3 sequence of SEQ ID NO: 37,
2) an antibody comprising the HVR-H1 sequence of SEQ ID NO: 38, the HVR-H2 sequence of SEQ ID NO: 39, the HVR-H3 sequence of SEQ ID NO: 40, the HVR-L1 sequence of SEQ ID NO: 41, the HVR-L2 sequence of SEQ ID NO: 42, and the HVR-L3 sequence of SEQ ID NO: 43,
3) an antibody comprising the HVR-H1 sequence of SEQ ID NO: 44, the HVR-H2 sequence of SEQ ID NO: 45, the HVR-H3 sequence of SEQ ID NO: 46, the HVR-L1 sequence of SEQ ID NO: 47, the HVR-L2 sequence of SEQ ID NO: 48, and the HVR-L3 sequence of SEQ ID NO: 49,
4) an antibody comprising the HVR-H1 sequence of SEQ ID NO: 50, the HVR-H2 sequence of SEQ ID NO: 51, the HVR-H3 sequence of SEQ ID NO: 52, the HVR-L1 sequence of SEQ ID NO: 53, the HVR-L2 sequence of SEQ ID NO: 54, and the HVR-L3 sequence of SEQ ID NO: 55, and
5) an antibody comprising the HVR-H1 sequence of SEQ ID NO: 56, the HVR-H2 sequence of SEQ ID NO: 57, the HVR-H3 sequence of SEQ ID NO: 58, the HVR-L1 sequence of SEQ ID NO: 59, the HVR-L2 sequence of SEQ ID NO: 60, and the HVR-L3 sequence of SEQ ID NO: 61, or
wherein the antibody specifically binds to an epitope within a CUB1-EGF-CUB2 domain of C1r , and competes for binding to the epitope with an antibody selected from the group consisting of 6)-13) below:
6) an antibody comprising the HVR-H1 sequence of SEQ ID NO:119, the HVR-H2 sequence of SEQ ID NO: 127, the HVR-H3 sequence of SEQ ID NO: 135, the HVR-L1 sequence of SEQ ID NO: 143, the HVR-L2 sequence of SEQ ID NO: 151, and the HVR-L3 sequence of SEQ ID NO: 159,
7) an antibody comprising the HVR-H1 sequence of SEQ ID NO: 120, the HVR-H2 sequence of SEQ ID NO: 128, the HVR-H3 sequence of SEQ ID NO: 136, the HVR-L1 sequence of SEQ ID NO: 144, the HVR-L2 sequence of SEQ ID NO: 152, and the HVR-L3 sequence of SEQ ID NO: 160,
8) an antibody comprising the HVR-H1 sequence of SEQ ID NO: 121, the HVR-H2 sequence of SEQ ID NO: 129, the HVR-H3 sequence of SEQ ID NO: 137, the HVR-L1 sequence of SEQ ID NO: 145, the HVR-L2 sequence of SEQ ID NO: 153, and the HVR-L3 sequence of SEQ ID NO: 161,
9) an antibody comprising the HVR-H1 sequence of SEQ ID NO: 122, the HVR-H2 sequence of SEQ ID NO: 130, the HVR-H3 sequence of SEQ ID NO: 138, the HVR-L1 sequence of SEQ ID NO: 146, the HVR-L2 sequence of SEQ ID NO: 154, and the HVR-L3 sequence of SEQ ID NO: 162,
10) an antibody comprising the HVR-H1 sequence of SEQ ID NO: 123, the HVR-H2 sequence of SEQ ID NO: 131, the HVR-H3 sequence of SEQ ID NO: 139, the HVR-L1 sequence of SEQ ID NO: 147, the HVR-L2 sequence of SEQ ID NO: 155, and the HVR-L3 sequence of SEQ ID NO: 163,
11) an antibody comprising the HVR-H1 sequence of SEQ ID NO: 124, the HVR-H2 sequence of SEQ ID NO: 132, the HVR-H3 sequence of SEQ ID NO: 140, the HVR-L1 sequence of SEQ ID NO: 148, the HVR-L2 sequence of SEQ ID NO: 156, and the HVR-L3 sequence of SEQ ID NO: 164,
12) an antibody comprising the HVR-H1 sequence of SEQ ID NO: 125, the HVR-H2 sequence of SEQ ID NO: 133, the HVR-H3 sequence of SEQ ID NO: 141, the HVR-L1 sequence of SEQ ID NO: 149, the HVR-L2 sequence of SEQ ID NO: 157, and the HVR-L3 sequence of SEQ ID NO: 165, and
13) an antibody comprising the HVR-H1 sequence of SEQ ID NO: 126, the HVR-H2 sequence of SEQ ID NO: 134, the HVR-H3 sequence of SEQ ID NO: 142, the HVR-L1 sequence of SEQ ID NO: 150, the HVR-L2 sequence of SEQ ID NO: 158, and the HVR-L3 sequence of SEQ ID NO: 166.

8. An isolated antibody that inhibits the interaction between C1q and C1r2s2 complex, wherein the antigen-binding activity of the antibody is lower at pH 5.8 than at pH 7.4.

9. The antibody of claim 1, wherein the antibody specifically binds to an epitope within a CUB1-EGF-CUB2 domain of C1s or C1r, wherein the antigen-binding activity of the antibody is lower at pH 5.8 than at pH 7.4.

10. The antibody of claim 9, wherein the antibody binds to C1s or C1r with a lower affinity at acidic pH than at neutral pH as described in (i) or (ii) below:
(i) when measured at a high calcium concentration at both neutral and acidic pH, the ratio of the KD value for C1s-binding activity at acidic pH to the KD value for C1s-binding activity at neutral pH (KD(acidic pH)/KD(neutral pH)) is 2 or more,
(ii) when measured at a high calcium concentration at neutral pH and at a low calcium concentration at acidic pH, the ratio of the KD value for C1s-binding activity at acidic pH to the KD value for C1s-binding activity at neutral pH (KD(acidic pH)/KD(neutral pH)) is 2 or more.

11. The antibody of claim 1, wherein the antibody comprises an Fc region that has at least one amino acid modification in the region so as to enhance the reduction of plasma antigen concentration and/or improve pharmacokinetics of the antibody.

12. The antibody of claim 11, wherein the antibody enhances the reduction of plasma antigen concentration, wherein the Fc region is a human Fc region that has a binding activity selected from the following group consisting of:
a) a binding activity to an activating Fc gamma receptor is stronger than the binding activity of an Fc region of the native human IgG1,
b) a binding activity to an inhibitory Fc gamma receptor is stronger than to an activating Fc gamma receptor, and
c) a binding activity to an FcRn at neutral pH is stronger than the binding activity of an Fc region of the native human IgG1.

13. The antibody of claim 1, wherein the antibody binds to both cynomolgus C1s and human C1s, or to both cynomolgus C1r and human C1r.

14. A pharmaceutical formulation comprising the antibody of claim 1 and a pharmaceutically acceptable carrier.

15. A method of treating an individual having a complement-mediated disease or disorder comprising administering to the individual an effective amount of the antibody of claim 1.

16. The antibody of claim 7, wherein the antibody specifically binds to an epitope within a CUB1-EGF-CUB2 domain of C1s or C1r, wherein the antigen-binding activity of the antibody is lower at pH 5.8 than at pH 7.4.

17. The antibody of claim 16, wherein the antibody binds to C1s or C1r with a lower affinity at acidic pH than at neutral pH as described in (i) or (ii) below:
(i) when measured at a high calcium concentration at both neutral and acidic pH, the ratio of the KD value for C1s-binding activity at acidic pH to the KD value for C1s-binding activity at neutral pH (KD(acidic pH)/KD(neutral pH)) is 2 or more,
(ii) when measured at a high calcium concentration at neutral pH and at a low calcium concentration at acidic pH, the ratio of the KD value for C1s-binding activity at acidic pH to the KD value for C1s-binding activity at neutral pH (KD(acidic pH)/KD(neutral pH)) is 2 or more.

18. The antibody of claim 16, wherein the antibody comprises an Fc region that has at least one amino acid modification in the region so as to enhance the reduction of plasma antigen concentration and/or improve pharmacokinetics of the antibody.

19. The antibody of claim 17, wherein the antibody comprises an Fc region that has at least one amino acid modification in the region so as to enhance the reduction of plasma antigen concentration and/or improve pharmacokinetics of the antibody.

20. The antibody of claim 7, wherein the antibody binds to both cynomolgus C1s and human C1s, or to both cynomolgus C1r and human C1r.

21. A pharmaceutical formulation comprising the antibody of claim 7 and a pharmaceutically acceptable carrier.

22. A method of treating an individual having a complement-mediated disease or disorder comprising administering to the individual an effective amount of the antibody of claim 17.

23. A method of treating an individual having a complement-mediated disease or disorder comprising administering to the individual an effective amount of the antibody of claim 22.

24. Isolated nucleic acids encoding the antibody of claim 1.

25. A host cell comprising the nucleic acids of claim 24.

26. A method of producing an antibody comprising culturing the host cell of claim 25 so that the antibody is produced.


SEQUENCE LISTING (167 sequences)

SEQ ID NO: 1 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 2 - protein, 688 aa, Rattus norvegicus
MWCFVFFSLLASFSAEPTMYGEILSPNYPQAYPNEVVKTWDIEVPEGFGIHLYFTHLDMELSENCAYDSVQIISGGIEEERLCGQRTSKSPNSPTVEEFQFPYNRLQVVFTSDFSNEERFTGFAAYYSAVDVNECTDFTDVPCSHFCNNFIGGYFCSCPPEYFLHDDMRTCGVNCSGDVFTALIGEIASPNYPNPYPENSRCEYQIRLQEGFRLVLTIRREDFDVEPADSEGNCHDSLTFAAKNQQFGPYCGNGFPGPLTIKTQSNTLDIVFQTDLTGQNKGWKLRYHGDPIPCPKEISANSIWEPEKAKYVFKDVVKITCVDGFEVVEGNVGSTSFYSTCQSNGQWSNSRLECQPVDCGVPEPIENGKVEDPEDTVFGSVIHYTCEEPYYYMEQEEGGEYHCAANGSWVNDQLGVELPKCIPVCGVPTEPFKVQQRIFGGYSTKIQSFPWQVYFESPRGGGALIDEYWVLTAAHVVEGNSDPVMYVGSTLLKIERLRNAQRLITERVIIHPSWKQEDDLNTRTNFDNDIALVQLKDPVKMGPTVAPICLPETSSDYNPSEGDLGLISGWGRTENRTNVIQLRGAKLPITSLEKCQQVKVENPKARSNDYVFTDNMICAGEKGVDSCEGDSGGAFALPVPNVKDPKFYVAGLVSWGKKCGTYGIYTKVKNYVDWILKTMQENSGPKKD

SEQ ID NO: 3 - protein, 688 aa, Macaca fascicularis
MWCIILFSLLAWVYAEPTMYGEILSPNYPQAYPSEVEKSWDIEVPEGYGIHLYFTHLDIELSENCAYDSVQIMSGDIEEGRLCGQRTSNNPYSPIVEEFQVPYNKLQVIFKSDFSNEERFTGFAAYYVATDINECTDFVDAPCSHFCNNFIGGYFCSCPPEYFLHDDMKNCGVNCSGDVFTALIGEIASPNYPKPYPENSRCEYQIRLEKGFQVVVTVRREDFDVEPADSEGNCLDSLVFVAGDQQFGPYCGRGFPGPLNIETKSNVLDIIFQTDLTGQNKGWKLRYHGDPMPCPKEETPTSVWEPAKAKYVFRDVVRITCLDGFEVVEGRVGATSFHSTCQSNGKWSNSKLKCQPVDCGIPESIENGKVEDPESTLFGSVTRYTCEEPYYYMENGGNGQYHCASNGSWVNEALSPELPKCVPVCGVPREPFEGKQRIIGGSDADIKNFPWQVFFDNPWAGGALIDEYWVLTAAHVVEGNQEPTMYVGSTSVQTSRLAKSKMLTSERVFIHPGWKLLEVPEARTNFDNDIALVQLKDPVKMGPTVAPICLPGTSSDYNLMDGDLGLIAGWGRTEKRDRALRLKAARLPVAPLRKCREVKVENPKADAGAYVFTPNMICAGGEKGMDSCKGDSGGAFAVQDPNDKTKFYVAGLVSWGPQCGTYGLYTRVQNYVDWIKKTMQENSTPSKD

SEQ ID NO: 4 - protein, 705 aa, Homo sapiens
MWLLYLLVPALFCRAGGSIPIPQKLFGEVTSPLFPKPYPNNFETTTVITVPTGYRVKLVFQQFDLEPSEGCFYDYVKISADKKSLGRFCGQLGSPLGNPPGKKEFMSQGNKMLLTFHTDFSNEENGTIMFYKGFLAYYQAVDLDECASRSKLGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 5 - protein, 705 aa, Macaca fascicularis
MWLLYLLVPALFCRAGGSIPIPQKLFGEVTSPLFPKPYPNSFETTTVITVPTGYRVKLVFQHFDLEPSEGCFYDYVKISADKKNLGRFCGQLGSPLGNPPGKKEFLSQGNKMLLTFHTDFSNEENGTIMFYKGFLAYYQAVDLDECASQSESGEKDPQPQCQHLCHNYVGGYFCSCRPGYELQEDRHSCQAECSSELYTEASGYISSLEYPRSYPPDLRCNYSIRVERGLTLHLKFLEPFEIDDHQQVHCPYDQLQIYANGKNIGEFCGKQRPPDFDTSSNAVDLLFFTDESGDSRGWKLRYTTEIIKCPQPKTLDEFTVIQNLQPQYQFRDYFIATCKLGYQLIEGNQVLHSFTAVCQDDGTWHRAMPRCKIKDCGQPRNLPNGAFRYTTTMGVNTYKARIQYYCHEPYYKMQTRAGSKESEQGLYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLANHPIRRVSIHPDYRQDESHNFEGDIALLELENSVTLGPNLLPICLPDNETFYDLGLMGYVSGFGVMEEKIAHDLRFVRLPVANRKDCETWLRGKNRLDVFSQNMFCAGHPSLKQDACQGDSGGVFAVRDPNTDRWIATGIVSWGIGCSKGYGFYTKVLNYVDWIKKEMEEED

SEQ ID NO: 6 - protein, 707 aa, Rattus norvegicus
MRLIPLLLVTLFCGVEGSIYLPQKLYGEVTSPLYPKPYPSDLETTTVITVPTGYRVKLVFWQFDVEPSEGCFYDYVKISADKKTLGRFCGQQDSPQGNPPGRKEFMSQGNKMLLTFHTDFSNEENGTIMFYKGFLAYYQAVDLDECTSQPNSVEEGLQPRCQHLCHNYVGGYFCSCRPGYELQKDRQSCQAECSSGLYTEPSGYISSLEYPKPYPPDLRCNYSIRVERGLTVHLKFLDPFEIDDHQQVHCPYDQLQIYANGKNLGEFCGTQRPPDLDTSSNAVDLLFFTDESGDSRGWKLRYTTEIIKCPQPKALDEFTIIQDPQPQYQFRDYFIVTCKQGYQLMEGSQALLSFTAVCQHDGTWHRAMPRCKIKDCGQPRSLSNGDFRYLTTRGVNTYEARIQYYCHDPYYKMMTRAGSSESVRGIYTCTPQGIWKNEEEGEKMPRCLPVCGKPVNPVTQKQRIIGGQKANPGNFPWQAFTNIHGRGGGALLGDRWILTAAHTIYPKEHNKENNVNVSKDVFLGHTNVEEIKKLGHHPVRRVIIHPDYRQDEPNNFEGDIALLELENSVTLGPNLLPICLPDNETFYDKDLMGYVSGFGITEDKIAFNLRFVRLPIADREACQRWLRTKNSNDVFSQNMFCSGDPTLKHDACQGDSGGVFAVRDRSRDIWVATGIVSWGIGCGEGYGFYTKLLNYVDWIKKEIGDEN

SEQ ID NO: 7 - protein, 701 aa, Artificial sequence
  an artificially synthesized sequence
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPREDGGGGSHHHHHHHH

SEQ ID NO: 8 - protein, 718 aa, Artificial sequence
  an artificially synthesized sequence
MWLLYLLVPALFCRAGGSIPIPQKLFGEVTSPLFPKPYPNNFETTTVITVPTGYRVKLVFQQFDLEPSEGCFYDYVKISADKKSLGRFCGQLGSPLGNPPGKKEFMSQGNKMLLTFHTDFSNEENGTIMFYKGFLAYYQAVDLDECASRSKLGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQQIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDAGGVFAVRDPNTDRWVATGIVSWGIGCSRGYGFYTKVLNYVDWIKKEMEEEDGGGGSDYKDDDDK

SEQ ID NO: 9 - protein, 701 aa, Artificial sequence
  an artificially synthesized sequence
MWCIILFSLLAWVYAEPTMYGEILSPNYPQAYPSEVEKSWDIEVPEGYGIHLYFTHLDIELSENCAYDSVQIMSGDIEEGRLCGQRTSNNPYSPIVEEFQVPYNKLQVIFKSDFSNEERFTGFAAYYVATDINECTDFVDAPCSHFCNNFIGGYFCSCPPEYFLHDDMKNCGVNCSGDVFTALIGEIASPNYPKPYPENSRCEYQIRLEKGFQVVVTVRREDFDVEPADSEGNCLDSLVFVAGDQQFGPYCGRGFPGPLNIETKSNVLDIIFQTDLTGQNKGWKLRYHGDPMPCPKEETPTSVWEPAKAKYVFRDVVRITCLDGFEVVEGRVGATSFHSTCQSNGKWSNSKLKCQPVDCGIPESIENGKVEDPESTLFGSVTRYTCEEPYYYMENGGNGQYHCASNGSWVNEALSPELPKCVPVCGVPREPFEGKQRIIGGSDADIKNFPWQVFFDNPWAGGALIDEYWVLTAAHVVEGNQEPTMYVGSTSVQTSRLAKSKMLTSERVFIHPGWKLLEVPEARTNFDNDIALVQLKDPVKMGPTVAPICLPGTSSDYNLMDGDLGLIAGWGRTEKRDRALRLKAARLPVAPLRKCREVKVENPKADAGAYVFTPNMICAGGEKGMDSCKGDSGGAFAVQDPNDKTKFYVAGLVSWGPQCGTYGLYTRVQNYVDWIKKTMQENSTPSKDGGGGSDYKDDDDK

SEQ ID NO: 10 - protein, 718 aa, Artificial sequence
  an artificially synthesized sequence
MWLLYLLVPALFCRAGGSIPIPQKLFGEVTSPLFPKPYPNSFETTTVITVPTGYRVKLVFQHFDLEPSEGCFYDYVKISADKKNLGRFCGQLGSPLGNPPGKKEFLSQGNKMLLTFHTDFSNEENGTIMFYKGFLAYYQAVDLDECASQSESGEKDPQPQCQHLCHNYVGGYFCSCRPGYELQEDRHSCQAECSSELYTEASGYISSLEYPRSYPPDLRCNYSIRVERGLTLHLKFLEPFEIDDHQQVHCPYDQLQIYANGKNIGEFCGKQRPPDFDTSSNAVDLLFFTDESGDSRGWKLRYTTEIIKCPQPKTLDEFTVIQNLQPQYQFRDYFIATCKLGYQLIEGNQVLHSFTAVCQDDGTWHRAMPRCKIKDCGQPRNLPNGAFRYTTTMGVNTYKARIQYYCHEPYYKMQTRAGSKESEQGLYTCTAQGIWKNEQKGEKIPRCLPVCGKPVNPVEQRQQIIGGQKAKMGNFPWQVFTNIHGRGGGALLGDRWILTAAHTLYPKEHEAQSNASLDVFLGHTNVEELMKLANHPIRRVSIHPDYRQDESHNFEGDIALLELENSVTLGPNLLPICLPDNETFYDLGLMGYVSGFGVMEEKIAHDLRFVRLPVANRKDCETWLRGKNRLDVFSQNMFCAGHPSLKQDACQGDAGGVFAVRDPNTDRWIATGIVSWGIGCSKGYGFYTKVLNYVDWIKKEMEEEDGGGGSHHHHHHHH

SEQ ID NO: 11 - protein, 19 aa, Artificial sequence
  an artificially synthesized sequence
MGWSCIILFLVATATGVHS

SEQ ID NO: 12 - protein, 429 aa, Artificial sequence
  an artificially synthesized sequence
MGWSCIILFLVATATGVHSMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPREDGGGGSHHHHHHHH

SEQ ID NO: 13 - protein, 701 aa, Artificial sequence
  an artificially synthesized sequence
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPREDGGGGSDYKDDDDK

SEQ ID NO: 14 - protein, 325 aa, Artificial sequence
  an artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 15 - protein, 328 aa, Artificial sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 16 - protein, 328 aa, Artificial sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCEVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 107 aa, Artificial sequence
  an artificially synthesized sequence
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 18 - protein, 118 aa, Artificial sequence
  an artificially synthesized sequence
EVMLVESGGGLVKPGGSLRLSCAASGFTFSNYAMSWVRQAPGKGLEWVATISSGGSHTYYLDSVKGRFTISRDNSKDTLYLQMNSLRAEDTALYYCARLFTGYAMDYWGQGTLVTVSS

SEQ ID NO: 19 - protein, 123 aa, Artificial sequence
  an artificially synthesized sequence
QSVEESGGRLVTPGTPLTLTCTVSGFSLSNYGMIWVRQAPGEGLEYIGFVSGSGSAYYANWVKGRFTISKTSTTVDLKMTSLTTEDTATYFCARGAYNTDSYSYSLYYGMDLWGPGTLVTVSS

SEQ ID NO: 20 - protein, 114 aa, Artificial sequence
  an artificially synthesized sequence
QSLEESGGRLVTPGTPLTLTCTASGFSLSNYNMAWVRQAPGKGLEWIGIIETTTNTYYARWARGRFTISKTSSTTVDLQIASPTTEDTATYFCGRRVSNSHDLWGPGTLVTVSS

SEQ ID NO: 21 - protein, 114 aa, Artificial sequence
  an artificially synthesized sequence
QSLEESGGRLVTPGTPLTLTCTASGFSLSSYHMDWVRQAPGKGLEWIGIITYTGDTYYATWAKGRFTISKTSSTTVDLKMASLTTEDTATYFCARRQYDAYNVWGPGTLVTVSS

SEQ ID NO: 22 - protein, 118 aa, Artificial sequence
  an artificially synthesized sequence
QSLEESGGRLVTPGTPLTLTCTVSGIDLSSYAMIWVRQAPGKGLEWIGIIYVRGSTYYASWAKDRFTISKTSTTVDLKITSPTTEDTATYFCARAYGGDTFSDGFDPWGPGTLVTVSS

SEQ ID NO: 23 - protein, 117 aa, Artificial sequence
  an artificially synthesized sequence
QSVEESGGRLVTPGTPLTLTCTVSGFSLSRYSMGWVRQAPGKGLEWIGIIYADGGAYYASWAKGRFTISKTSTTMDLKVTSPTTEDTATYFCARGQFINSNEFFNPWGPGTLVTVSS

SEQ ID NO: 24 - protein, 117 aa, Artificial sequence
  an artificially synthesized sequence
QSLEESGGRLVTPGTPLTLTCTASGFSLSAYAMNWVRQAPGKGLEWIGLIYGSGSTFYASWAKGRFTISKTSTTVDLKMTSLTTEDTATYFCARGRSVNYNSDFKLWGPGTLVTVSS

SEQ ID NO: 25 - protein, 109 aa, Artificial sequence
  an artificially synthesized sequence
QIVLTQSPATLSLSPGERATLSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 26 - protein, 110 aa, Artificial sequence
  an artificially synthesized sequence
QIVMTQTPASVEVAVGGTVTIKCQASQSIYSYLSWYQQKPGQPPKLLIYGASTLASGVPSRFKGSRSGTEFTLTISDLECADAATYYCQSNYATTTTYGTFGGGTKVEIK

SEQ ID NO: 27 - protein, 111 aa, Artificial sequence
  an artificially synthesized sequence
AYDMTQTPASVSAAVGGTVTINCQASQTIGGWCAWYQQKPGQPPKLLIYKASTLASGVSSRFKGSGSGTEFALTISDLECDDAATYYCQHGYYTSSTIENTFGGGTKVEIK

SEQ ID NO: 28 - protein, 111 aa, Artificial sequence
  an artificially synthesized sequence
AYDMTQTPASVEVAVGGTVTIKCQASQSINNYLAWYQQKPGQPPKLLIYYASTLASGVPSRFKGSRSGTEFTLTISGVECADAATYYCQHGYYTSSNIDSVFGGGTKVEIK

SEQ ID NO: 29 - protein, 110 aa, Artificial sequence
  an artificially synthesized sequence
DPVMTQTPSSTSAAVGGTVTINCQSSQNVYGNNRLAWYQQKPGQPPKLLIYLASTLASGVPSRFRGNGSGTQFTLTISGVQCDDAATYYCAGGYRDDTNSFGGGTKVEIK

SEQ ID NO: 30 - protein, 112 aa, Artificial sequence
  an artificially synthesized sequence
QVLTQTASSVSAVVGGTVTINCQASQSLHNNKNLAWYQQKPGQPPRLLIYYASTLASGVSSRFKGSGSGTQFTLTISDLECDDAATYYCAGEFSCSNGDCNAFGGGTKVEIK

SEQ ID NO: 31 - protein, 112 aa, Artificial sequence
  an artificially synthesized sequence
QVLTQTPSSVSAAVGGTVTINCQASQSLHDKKNLAWYQHKPGQRPNLLIYGASTLESGVPSRFKGSGSGTEYTLTISDLECDDAAAYYCQGEFSCSSADCNTFGGGTKVEIK

SEQ ID NO: 32 - protein, 5 aa, Artificial sequence
  an artificially synthesized sequence
NYGMI

SEQ ID NO: 33 - protein, 16 aa, Artificial sequence
  an artificially synthesized sequence
FVSGSGSAYYANWVKG

SEQ ID NO: 34 - protein, 18 aa, Artificial sequence
  an artificially synthesized sequence
GAYNTDSYSYSLYYGMDL

SEQ ID NO: 35 - protein, 11 aa, Artificial sequence
  an artificially synthesized sequence
QASQSIYSYLS

SEQ ID NO: 36 - protein, 7 aa, Artificial sequence
  an artificially synthesized sequence
GASTLAS

SEQ ID NO: 37 - protein, 12 aa, Artificial sequence
  an artificially synthesized sequence
QSNYATTTTYGT

SEQ ID NO: 38 - protein, 5 aa, Artificial sequence
  an artificially synthesized sequence
NYNMA

SEQ ID NO: 39 - protein, 16 aa, Artificial sequence
  an artificially synthesized sequence
IIETTTNTYYARWARG

SEQ ID NO: 40 - protein, 8 aa, Artificial sequence
  an artificially synthesized sequence
RVSNSHDL

SEQ ID NO: 41 - protein, 11 aa, Artificial sequence
  an artificially synthesized sequence
QASQTIGGWCA

SEQ ID NO: 42 - protein, 7 aa, Artificial sequence
  an artificially synthesized sequence
KASTLAS

SEQ ID NO: 43 - protein, 13 aa, Artificial sequence
  an artificially synthesized sequence
QHGYYTSSTIENT

SEQ ID NO: 44 - protein, 5 aa, Artificial sequence
  an artificially synthesized sequence
SYHMD

SEQ ID NO: 45 - protein, 16 aa, Artificial sequence
  an artificially synthesized sequence
IITYTGDTYYATWAKG

SEQ ID NO: 46 - protein, 8 aa, Artificial sequence
  an artificially synthesized sequence
RQYDAYNV

SEQ ID NO: 47 - protein, 11 aa, Artificial sequence
  an artificially synthesized sequence
QASQSINNYLA

SEQ ID NO: 48 - protein, 7 aa, Artificial sequence
  an artificially synthesized sequence
YASTLAS

SEQ ID NO: 49 - protein, 13 aa, Artificial sequence
  an artificially synthesized sequence
QHGYYTSSNIDSV

SEQ ID NO: 50 - protein, 5 aa, Artificial sequence
  an artificially synthesized sequence
RYSMG

SEQ ID NO: 51 - protein, 16 aa, Artificial sequence
  an artificially synthesized sequence
IIYADGGAYYASWAKG

SEQ ID NO: 52 - protein, 12 aa, Artificial sequence
  an artificially synthesized sequence
GQFINSNEFFNP

SEQ ID NO: 53 - protein, 13 aa, Artificial sequence
  an artificially synthesized sequence
QASQSLHNNKNLA

SEQ ID NO: 54 - protein, 7 aa, Artificial sequence
  an artificially synthesized sequence
YASTLAS

SEQ ID NO: 55 - protein, 13 aa, Artificial sequence
  an artificially synthesized sequence
AGEFSCSNGDCNA

SEQ ID NO: 56 - protein, 5 aa, Artificial sequence
  an artificially synthesized sequence
AYAMN

SEQ ID NO: 57 - protein, 16 aa, Artificial sequence
  an artificially synthesized sequence
LIYGSGSTFYASWAKG

SEQ ID NO: 58 - protein, 12 aa, Artificial sequence
  an artificially synthesized sequence
GRSVNYNSDFKL

SEQ ID NO: 59 - protein, 13 aa, Artificial sequence
  an artificially synthesized sequence
QASQSLHDKKNLA

SEQ ID NO: 60 - protein, 7 aa, Artificial sequence
  an artificially synthesized sequence
GASTLES

SEQ ID NO: 61 - protein, 13 aa, Artificial sequence
  an artificially synthesized sequence
QGEFSCSSADCNT

SEQ ID NO: 62 - protein, 115 aa, Artificial sequence
  an artificially synthesized sequence
QSVEESGGRLVTPGTPLTLTCTVSGFSLTKYTVSWVRQAPGKGLEWIGIINTGGSAYYATWAKGRFTFSKTSTTVDLQITSPTTEDTATYFCARGNGDTDYTNLWGPGTLVTVSS

SEQ ID NO: 63 - protein, 117 aa, Artificial sequence
  an artificially synthesized sequence
QSVEESGGRLVTPGTPLTLTCTVSGFSLSSYPMGWVRQVPGEGLEWIGTISAGGSTYYPSWAKGRFTISRTSTTVDLKMTSLTTEDTATYFCARGYPYTDGTYMTLWGPGTLVTVSS

SEQ ID NO: 64 - protein, 118 aa, Artificial sequence
  an artificially synthesized sequence
QSVEESGGRLVTPGTPLTLTCTVSGIDLNNYPMGWVRQAPGKGLEYIGIISSSGGTSYASWAKGRFTISKTSSTTVDLKMTSLTTEDTATYFCARGYPYRDITYFNLWGPGTLVTVSS

SEQ ID NO: 65 - protein, 113 aa, Artificial sequence
  an artificially synthesized sequence
QSLEESGGRLVTPGTPLTLTCTVSGFSLSSYTMIWVRQAPGKGLEWIGVVGGSGITYYASWAKGRFTISKTSTTVDLQITSPTTEDTATYFCARDTSVAGDLWGPGTLVTVSS

SEQ ID NO: 66 - protein, 117 aa, Artificial sequence
  an artificially synthesized sequence
QSVEESGGRLVTPGGSLTLTCTVSGFSLSRNAINWVRQAPGKGLEWIGGVSGGATTYYASWANGRFTISKTSTTVDLKMTGLTAADTATYFCARGAGSNIDGPFNLWGPGTLVTVSS

SEQ ID NO: 67 - protein, 106 aa, Artificial sequence
  an artificially synthesized sequence
QVLTQTPSSVSEPVGGTVTINCQASENIYSALAWYQQKPGQPPKLLIYGASNLESGVPSRFKGSGSGTQFTLTISDLECADAATYYCQQYYFTSSTFGGGTKVEIK

SEQ ID NO: 68 - protein, 108 aa, Artificial sequence
  an artificially synthesized sequence
AFELTQTPSSVEAAVGGTVTIKCQASENIYSLLAWYQQKPGQPPKLLIYGASTLASGVSSRFSGSGYGTEFTLTISDLECADAATYYCQSYYDSSTTTFGGGTKVEIK

SEQ ID NO: 69 - protein, 110 aa, Artificial sequence
  an artificially synthesized sequence
DPVLTQTPSPVSAPVGGSVTIKCQASQNIYSYLSWYQHKPGQPPKLLIYGASTLTNGVSSRFTGSGYGTEFTLTISDLECEDAATYYCQNYYSGGSADTTFGGGTKVEIK

SEQ ID NO: 70 - protein, 110 aa, Artificial sequence
  an artificially synthesized sequence
AYDMTQTPASVEVAVGGTVTIKCQASQSIGVSLAWYQQKPGQRPKLLIYKASNLASGVPSRFKGSGSGTQFTLTISGVECADAATYYCQQDYTYSDVTNIFGGGTKVEIK

SEQ ID NO: 71 - protein, 108 aa, Artificial sequence
  an artificially synthesized sequence
AFEMTQTPSSVSAPVGGTVTIKCQASQNIYSYLSWYQQKPGHPPKLLIYGASTLASGVPSRFKGSGYGTEFTLTISDLECADPATYYCQQYYATTSVDFGGGTKVEIK

SEQ ID NO: 72 - protein, 5 aa, Artificial sequence
  an artificially synthesized sequence
KYTVS

SEQ ID NO: 73 - protein, 16 aa, Artificial sequence
  an artificially synthesized sequence
IINTGGSAYYATWAKG

SEQ ID NO: 74 - protein, 10 aa, Artificial sequence
  an artificially synthesized sequence
GNGDTDYTNL

SEQ ID NO: 75 - protein, 5 aa, Artificial sequence
  an artificially synthesized sequence
SYPMG

SEQ ID NO: 76 - protein, 16 aa, Artificial sequence
  an artificially synthesized sequence
TISAGGSTYYPSWAKG

SEQ ID NO: 77 - protein, 12 aa, Artificial sequence
  an artificially synthesized sequence
GYPYTDGTYMTL

SEQ ID NO: 78 - protein, 5 aa, Artificial sequence
  an artificially synthesized sequence
NYPMG

SEQ ID NO: 79 - protein, 16 aa, Artificial sequence
  an artificially synthesized sequence
IISSSGGTSYASWAKG

SEQ ID NO: 80 - protein, 12 aa, Artificial sequence
  an artificially synthesized sequence
GYPYRDITYFNL

SEQ ID NO: 81 - protein, 5 aa, Artificial sequence
  an artificially synthesized sequence
SYTMI

SEQ ID NO: 82 - protein, 16 aa, Artificial sequence
  an artificially synthesized sequence
VVGGSGITYYASWAKG

SEQ ID NO: 83 - protein, 8 aa, Artificial sequence
  an artificially synthesized sequence
DTSVAGDL

SEQ ID NO: 84 - protein, 5 aa, Artificial sequence
  an artificially synthesized sequence
RNAIN

SEQ ID NO: 85 - protein, 16 aa, Artificial sequence
  an artificially synthesized sequence
GVSGGATTYYASWANG

SEQ ID NO: 86 - protein, 12 aa, Artificial sequence
  an artificially synthesized sequence
GAGSNIDGPFNL

SEQ ID NO: 87 - protein, 11 aa, Artificial sequence
  an artificially synthesized sequence
QASENIYSALA

SEQ ID NO: 88 - protein, 7 aa, Artificial sequence
  an artificially synthesized sequence
GASNLES

SEQ ID NO: 89 - protein, 9 aa, Artificial sequence
  an artificially synthesized sequence
QQYYFTSST

SEQ ID NO: 90 - protein, 11 aa, Artificial sequence
  an artificially synthesized sequence
QASENIYSLLA

SEQ ID NO: 91 - protein, 7 aa, Artificial sequence
  an artificially synthesized sequence
GASTLAS

SEQ ID NO: 92 - protein, 10 aa, Artificial sequence
  an artificially synthesized sequence
QSYYDSSTTT

SEQ ID NO: 93 - protein, 11 aa, Artificial sequence
  an artificially synthesized sequence
QASQNIYSYLS

SEQ ID NO: 94 - protein, 7 aa, Artificial sequence
  an artificially synthesized sequence
GASTLTN

SEQ ID NO: 95 - protein, 12 aa, Artificial sequence
  an artificially synthesized sequence
QNYYSGGSADTT

SEQ ID NO: 96 - protein, 11 aa, Artificial sequence
  an artificially synthesized sequence
QASQSIGVSLA

SEQ ID NO: 97 - protein, 7 aa, Artificial sequence
  an artificially synthesized sequence
KASNLAS

SEQ ID NO: 98 - protein, 12 aa, Artificial sequence
  an artificially synthesized sequence
QQDYTYSDVTNI

SEQ ID NO: 99 - protein, 11 aa, Artificial sequence
  an artificially synthesized sequence
QASQNIYSYLS

SEQ ID NO: 100 - protein, 7 aa, Artificial sequence
  an artificially synthesized sequence
GASTLAS

SEQ ID NO: 101 - protein, 10 aa, Artificial sequence
  an artificially synthesized sequence
QQYYATTSVD

SEQ ID NO: 102 - protein, 107 aa, Artificial sequence; identical to SEQ ID NO: 17
  an artificially synthesized sequence

SEQ ID NO: 103 - protein, 117 aa, Artificial Sequence
  an artificially synthesized sequence
QSLEESGGRLVTPGTPLTLTCTASGFTFSGYYMCWVRQTPGKGLEYIGIIASSGNTYYANWAKGRFTISKTSSTTVDLKMTSLTTEDTATYFCAKPESDYVNGMGLWGPGTLVTVSS

SEQ ID NO: 104 - protein, 117 aa, Artificial Sequence
  an artificially synthesized sequence
QSVEESGGRLVTPGTPLTLTCTASGFDISNYNMQWVRQSPGKGLEWIGIIWSGGYTDYANWAKGRFTISATSTTVDLKSTSPTTEDTATYFCARQGANFGDYTVDLWGPGTLVTVSS

SEQ ID NO: 105 - protein, 118 aa, Artificial sequence
  an artificially synthesized sequence
QSLEESGGGLVQPEGSLTLTCKASGIDFSSNAMCWVRQAPGKGLEWIGCIYTGSTTIWYASWAKGRFTISKTSSTTVTLQLNSLTVADTATYFCARRDSSSGYAFKLWGPGTLVTVSS

SEQ ID NO: 106 - protein, 110 aa, Artificial sequence
  an artificially synthesized sequence
QSLEESGGGLFKPTDTLTLTCTVSGFSLSSNAMSWVRQAPGKGLEYIGIIYLTGVTYYATWAKGRFTISKTSTTVDLKITSPTTEDTATYFCAGGEFSLWGPGTLVTVSS

SEQ ID NO: 107 - protein, 118 aa, Artificial sequence
  an artificially synthesized sequence
QSVEESGGRLVTPGTPLTLTCTVSGFSLSNYYMNWVRQAPGKGLEWIGIIASDDNTWYASWAKGRFTISKTSTTVDLKITSPTTEDTATYFCARDLDVGYGSGWYDLWGQGTLVTVSS

SEQ ID NO: 108 - protein, 114 aa, Artificial sequence
  an artificially synthesized sequence
QEQLKESGGGLVTPGGTLTLTCTASGFSLSSYDMSWVRQAPGKGLDWIGIINTGGSAYYATWAKGRFTISKTSTTVDLKITSPTTEDTATYFCGRKINDAFDPWGPGTLVTVSS

SEQ ID NO: 109 - protein, 119 aa, Artificial sequence
  an artificially synthesized sequence
QSLEESGGRLVTPGTPLTLTCTASGFSLSSYDMTWVRQAPGKGLEWIGIIYTLGSAYYASWAKGRFTISRTSTTVDLKMTSPTTEDTATYFCARGGDSYGAVDTAFDLWGPGTLVTVSS

SEQ ID NO: 110 - protein, 118 aa, Artificial sequence
  an artificially synthesized sequence
QSLEESGGGLVQPEGSLTLTCKASGIDFSSNAMCWVRQAPGKGLEWIACIYTGSTTIWYATWAKGRFTISKTSSTTVTLQLNSLTVADTATYFCARRASSSGYAFKLWGPGTLVTVSS

SEQ ID NO: 111 - protein, 114 aa, Artificial sequence
  an artificially synthesized sequence
DIVMTQTPASVSDPVGGTVTIKCQASQSIYNLLSWYQQKPGQRPKLLIYDASKLASGVPSRFKGSGYGTDFTLTISDLGCADAATYYCQSNYGYSRTDDTFFYGFGGGTKVEIK

SEQ ID NO: 112 - protein, 110 aa, Artificial sequence
  an artificially synthesized sequence
AYDMTQTPASVEVAVGGTVTIKCQASQSIRSSLAWYQQKPGQRPKLLIYRASTLASGVSSRFKGSGSGTQFTLTISGVECADAATYYCQQGYSNINVDNTFGGGTKVEIK

SEQ ID NO: 113 - protein, 107 aa, Artificial sequence
  an artificially synthesized sequence
DIVMTQTPSSVSAAVGGTVTIKCQASENIYSNLAWYQQKSGQPPNLLIYGASTLESGVPSRFKGSGSGTEFTLTISDLECADAAIYYCQCTYDDSSTFGGGTKVEIK

SEQ ID NO: 114 - protein, 113 aa, Artificial sequence
  an artificially synthesized sequence
DIIMTQTPASVEAAVGGTVTIKCQASQGIDNYFSWHQQKPGQPPKLLIYKASTLASGVSSRFKGSGSGTEFTLTISDLECDDAATYYCQCTYGYNINDNYGNAFGGGTKVEIK

SEQ ID NO: 115 - protein, 111 aa, Artificial sequence
  an artificially synthesized sequence
AAVLTQTPSPVSVAVGGTVTINCQASQSVYNNNYLAWYQQEPGHRPKLLIYRASKLASGVPSRFSGSGSGTQFTLTISGVQCDDAATYYCLGGYDDFTDTTFGGGTKVEIK

SEQ ID NO: 116 - protein, 111 aa, Artificial sequence
  an artificially synthesized sequence
AQVLTQTPFSVSAAVGGTVTINCQASESISSNLAWYQQKPGQPPKLLIYDASDLASGVPSRFKGSGSGKQFTLTISGVQCDDAATYFCQHGWIYDTIDSNCFGGGTKVEIK

SEQ ID NO: 117 - protein, 109 aa, Artificial sequence
  an artificially synthesized sequence
AQVLTQTPSPVSAAVGGTVTISCQSSPSVYSNYLSWYQQKPGQPPKLLIYRASRLASGVPSRFKGSGSGTQFTLTISEVQCDDAATYYCQGYFGNGINAFGGGTKVEIK

SEQ ID NO: 118 - protein, 107 aa, Artificial sequence
  an artificially synthesized sequence
DVVMTQTPFSVSAAVGGTVTIKCQASESIYSNLAWYQQKPGQPPNLLIYGASTLESGVPSRFKGSGSGTEFTLTISDLECDDAAIYYCQCTYDDSSTFGGGTKVEIK

SEQ ID NO: 119 - protein, 5 aa, Artificial sequence
  an artificially synthesized sequence
GYYMC

SEQ ID NO: 120 - protein, 5 aa, Artificial sequence
  an artificially synthesized sequence
NYNMQ

SEQ ID NO: 121 - protein, 5 aa, Artificial sequence
  an artificially synthesized sequence
SNAMC

SEQ ID NO: 122 - protein, 5 aa, Artificial sequence
  an artificially synthesized sequence
SNAMS

SEQ ID NO: 123 - protein, 5 aa, Artificial sequence
  an artificially synthesized sequence
NYYMN

SEQ ID NO: 124 - protein, 5 aa, Artificial sequence
  an artificially synthesized sequence
SYDMS

SEQ ID NO: 125 - protein, 5 aa, Artificial sequence
  an artificially synthesized sequence
SYDMT

SEQ ID NO: 126 - protein, 5 aa, Artificial sequence
  an artificially synthesized sequence
SNAMC

SEQ ID NO: 127 - protein, 16 aa, Artificial sequence
  an artificially synthesized sequence
IIASSGNTYYANWAKG

SEQ ID NO: 128 - protein, 16 aa, Artificial sequence
  an artificially synthesized sequence
IIWSGGYTDYANWAKG

SEQ ID NO: 129 - protein, 17 aa, Artificial sequence
  an artificially synthesized sequence
CIYTGSTTIWYASWAKG

SEQ ID NO: 130 - protein, 16 aa, Artificial sequence
  an artificially synthesized sequence
IIYLTGVTYYATWAKG

SEQ ID NO: 131 - protein, 16 aa, Artificial sequence
  an artificially synthesized sequence
IIASDDNTWYASWAKG

SEQ ID NO: 132 - protein, 15 aa, Artificial sequence
  an artificially synthesized sequence
IINTGGSAYYATWAK

SEQ ID NO: 133 - protein, 16 aa, Artificial sequence
  an artificially synthesized sequence
IIYTLGSAYYASWAKG

SEQ ID NO: 134 - protein, 17 aa, Artificial sequence
  an artificially synthesized sequence
CIYTGSTTIWYATWAKG

SEQ ID NO: 135 - protein, 11 aa, Artificial sequence
  an artificially synthesized sequence
PESDYVNGMGL

SEQ ID NO: 136 - protein, 12 aa, Artificial sequence
  an artificially synthesized sequence
QGANFGDYTVDL

SEQ ID NO: 137 - protein, 11 aa, Artificial sequence
  an artificially synthesized sequence
RDSSSGYAFKL

SEQ ID NO: 138 - protein, 5 aa, Artificial sequence
  an artificially synthesized sequence
GEFSL

SEQ ID NO: 139 - protein, 13 aa, Artificial sequence
  an artificially synthesized sequence
DLDVGYGSGWYDL

SEQ ID NO: 140 - protein, 8 aa, Artificial sequence
  an artificially synthesized sequence
KINDAFDP

SEQ ID NO: 141 - protein, 14 aa, Artificial sequence
  an artificially synthesized sequence
GGDSYGAVDTAFDL

SEQ ID NO: 142 - protein, 11 aa, Artificial sequence
  an artificially synthesized sequence
RASSSGYAFKL

SEQ ID NO: 143 - protein, 11 aa, Artificial sequence
  an artificially synthesized sequence
QASQSIYNLLS

SEQ ID NO: 144 - protein, 11 aa, Artificial sequence
  an artificially synthesized sequence
QASQSIRSSLA

SEQ ID NO: 145 - protein, 11 aa, Artificial sequence
  an artificially synthesized sequence
QASENIYSNLA

SEQ ID NO: 146 - protein, 11 aa, Artificial sequence
  an artificially synthesized sequence
QASQGIDNYFS

SEQ ID NO: 147 - protein, 13 aa, Artificial sequence
  an artificially synthesized sequence
QASQSVYNNNYLA

SEQ ID NO: 148 - protein, 11 aa, Artificial sequence
  an artificially synthesized sequence
QASESISSNLA

SEQ ID NO: 149 - protein, 12 aa, Artificial sequence
  an artificially synthesized sequence
QSSPSVYSNYLS

SEQ ID NO: 150 - protein, 11 aa, Artificial sequence
  an artificially synthesized sequence
QASESIYSNLA

SEQ ID NO: 151 - protein, 7 aa, Artificial sequence
  an artificially synthesized sequence
DASKLAS

SEQ ID NO: 152 - protein, 7 aa, Artificial sequence
  an artificially synthesized sequence
RASTLAS

SEQ ID NO: 153 - protein, 7 aa, Artificial sequence
  an artificially synthesized sequence
GASTLES

SEQ ID NO: 154 - protein, 7 aa, Artificial sequence
  an artificially synthesized sequence
KASTLAS

SEQ ID NO: 155 - protein, 7 aa, Artificial sequence
  an artificially synthesized sequence
RASKLAS

SEQ ID NO: 156 - protein, 7 aa, Artificial sequence
  an artificially synthesized sequence
DASDLAS

SEQ ID NO: 157 - protein, 7 aa, Artificial sequence
  an artificially synthesized sequence
RASRLAS

SEQ ID NO: 158 - protein, 7 aa, Artificial sequence
  an artificially synthesized sequence
GASTLES

SEQ ID NO: 159 - protein, 16 aa, Artificial sequence
  an artificially synthesized sequence
QSNYGYSRTDDTFFYG

SEQ ID NO: 160 - protein, 12 aa, Artificial sequence
  an artificially synthesized sequence
QQGYSNINVDNT

SEQ ID NO: 161 - protein, 9 aa, Artificial sequence
  an artificially synthesized sequence
QCTYDDSST

SEQ ID NO: 162 - protein, 15 aa, Artificial sequence
  an artificially synthesized sequence
QCTYGYNINDNYGNA

SEQ ID NO: 163 - protein, 11 aa, Artificial sequence
  an artificially synthesized sequence
LGGYDDFTDTT

SEQ ID NO: 164 - protein, 13 aa, Artificial sequence
  an artificially synthesized sequence
QHGWIYDTIDSNC

SEQ ID NO: 165 - protein, 10 aa, Artificial sequence
  an artificially synthesized sequence
QGYFGNGINA

SEQ ID NO: 166 - protein, 9 aa, Artificial sequence
  an artificially synthesized sequence
QCTYDDSST

SEQ ID NO: 167 - protein, 431 aa, Artificial sequence
  an artificially synthesized sequence
MGWSCIILFLVATATGVHSIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQQIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDAGGVFAVRDPNTDRWVATGIVSWGIGCSRGYGFYTKVLNYVDWIKKEMEEEDGGGGSDYKDDDDK